(19) United States
(12) Patent Application Publication
(10) Pub. No.: US 2025/0387334 A1
(43) Pub. Date: Dec. 25, 2025

(54) Oxyanion azide-benzaldehyde acetal acid-degradable lipids

(71) Applicant: The Regents of the University of California, Oakland, CA (US)

(72) Inventors: Niren Murthy, Berkeley, CA (US); Hesong Han, Berkeley, CA (US); Sheng Zhao, Berkeley, CA (US)

(73) Assignee: The Regents of the University of California, Oakland, CA (US)

(21) Appl. No.: 19/308,239

(22) Filed: Aug. 24, 2025

Related U.S. Application Data
(63) Continuation of application No. PCT/US2024/019258, filed on Mar. 8, 2024.
(60) Provisional application No. 63/489,180, filed on Mar. 8, 2023.
(60) Provisional application No. 63/489,181, filed on Mar. 8, 2023.

(51) Int. Cl. A61K 9/14; A61K 31/7105; A61K 31/711; A61K 39/00; A61K 48/00; C07K 1/00; C07K 4/00

(52) U.S. Cl. CPC A61K 9/145; A61K 31/7105; A61K 31/711; A61K 39/00; A61K 48/0033; C07K 1/00; C07K 4/00; A61K 2039/53

(57) ABSTRACT

Compounds comprising an oxyanion azide-benzaldehyde acetal acid-degradable lipid are incorporated in lipid nanoparticle (LNP) and used to transfect cells.

### CROSS-REFERENCES TO RELATED APPLICATIONS

This application is a continuation of PCT/US24/19258; filed Mar. 8, 2024, which claims priority to U.S. Provisional Application No. 63/489,180; filed Mar. 8, 2023 and to U.S. Provisional Application No. 63/489,181; filed Mar. 8, 2023, the disclosures of which are hereby incorporated by reference in its entirety for all purposes.

### INTRODUCTION

LNP-based delivery vectors are revolutionizing medicine. However, LNP/mRNA complexes have several limitations that prevent them from serving as a platform for developing new therapeutics. For example, LNP/mRNA complexes only deliver 1-4% of their mRNA cargo into the cytoplasm¹. The low endosomal disruption rate of LNPs restricts their medical applications because it requires delivering large amounts of mRNA to cells to generate therapeutic quantities of proteins. The liver is the only organ that internalizes LNPs robustly enough to compensate for the low endosomal disruption rates of LNPs, and developing LNP/mRNA complexes for non-liver diseases will require engineering LNPs to efficiently disrupt endosomes². In addition, LNPs exhibit high levels of toxicity, due to their cationic and ionizable lipids and also persist in tissues for days to weeks³. These issues prevent LNP/mRNA complexes from being chronically administered and also limit their dose. Collectively, the low endosomal disruption rate, toxicity, and tissue persistence of LNP/mRNA complexes prevent them from treating a wide variety of devastating diseases, and strategies for improving the performance of LNPs are greatly needed.

LNPs that degrade in endosomes within 30 minutes (termed RD-LNPs) have the potential to address several of the limitations of conventional LNPs. In particular, endosomal trafficking to lysosomes occurs on a timescale of 30 minutes and RD-LNPs will degrade in endosomes before fusion with lysosomes occurs. The rapid hydrolysis of the RD-LNPs should enable them to trigger endosomal disruption and release of mRNA into the cytoplasm before mRNA degradation in the lysosomes occurs⁴. In addition, RD-LNPs can get cleared from cells faster than conventional LNPs and this should lower toxicity and accumulation within tissues.

Acid-degradable lipids have great potential for generating RD-LNPs, given the pH gradient between the endosome and the blood. For example, early endosomes have a pH of 6.0, and late endosomes have a pH of 5.0, whereas the blood is at pH 7.4. There is consequently great interest in developing acid-degradable lipids (ADLs) and using them to formulate RD-LNPs⁵⁻⁹. ADLs have been synthesized with linkages based on vinyl ethers¹⁰, orthoesters¹¹, ketals¹², acetals^{2,13,14}, and hydrazones¹⁵ and have been incorporated into liposomes, cationic liposomes, and LNPs and have generated promising results. However, existing acid-degradable lipids degrade on the time-scale of days at pH 6.0-6.8 and on the timescale of hours at pH 5.0-6.0, and are consequently trafficked to lysosomes, where they are degraded. Developing acid-degradable lipids that rapidly hydrolyze at pH 6.0 has been challenging because the hydrolysis rate of conventional acid-degradable linkers is proportional to the hydronium ion concentration, which only changes by a factor of 5-15 between the pHs of 7.4 and 6.0-6.8¹⁶. Consequently, a linker that hydrolyzes within 30 minutes at pH 6.0-6.8 would also rapidly hydrolyze at pH 7.4and will be too unstable for synthesizing acid-degradable lipids or their formulation into LNPs.

Relevent literature includes: Knorr et al., Bioconjugate Chem. 2007, 18, 4, 1218-1225; Liu et al., J. Am. Chem. Soc. 2017, 139, 6, 2306-2317.

### SUMMARY OF THE INVENTION

The invention provides compounds comprising an oxyanion azide-benzaldehyde acetal acid-degradable lipid that can be incorporated in lipid nanoparticle (LNP) and used to transfect cells.

In an aspect the invention provides acid-degradable linkers, termed an azido-acetals that hydrolyze in endosomes within minutes and enable the production of RD-LNPs. Acid-degradable lipids composed of PEG-lipids (1), anionic lipids (2), and cationic lipids (3) are synthesized with the azido-acetal linker and used to generate RD-LNPs, which significantly improve the performance of LNP/mRNA complexes in vitro and in vivo. RD-LNPs deliver mRNA more efficiently to organs and to hematopoietic stem/progenitor cells (HSPCs) than conventional LNPs.

In aspects and embodiments, the invention provides:
1. A composition comprising a compound comprising an oxyanion azide-benzaldehyde acetal acid-degradable lipid of structure:

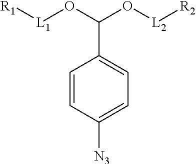

R1 comprises an acyl moiety comprising n (1-5) oxyanions (such as carbonic, phosphoric, sulfonic etc.), each complexed with a metal cation (such as Na⁺, K⁺, Li⁺, etc.)

R2 comprises a hydrophobic group or lipid, such as a steroid (e.g. cholesterol) or one or more alkyl chains, such as in a single chain fatty acid or double or triple chain fatty acid ester, that can strengthen the rigidity of a lipid nanoparticle;

L1 and L2 are linkers selected from a bond, an optionally substituted heteroatom and an optionally substituted C1-18 hydrocarbyl or heterohydrocarbyl, providing acid degradable linkages.
2. The composition of claim 1 wherein R1 comprises a framework of a:
C1-C6 alkyl-substituted heteroatom (N, O or S),
C1-C18 linear or branched alkyl or heteroalkyl,
C3-C6 cycloalkyl or cycloheteroalkyl, or
C5-C6 aryl or heteroaryl.
3. The composition of claim 1 wherein R1 is comprises n (1-5) acetate groups (e.g. of structure -COR(COOM)n);
4. The composition of claim 1, wherein R1 comprises:

C1-C6 alkyl-substituted heteroatom (N, O or S), such as:

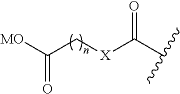

n=1−4, X=N, O, S, such as:

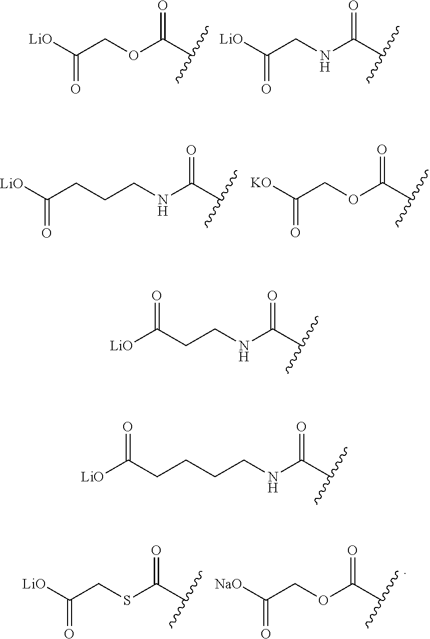

5. The composition of claim 1, wherein R1 comprises:

C1-C18 linear or branched alkyl or heteroalkyl, such as:

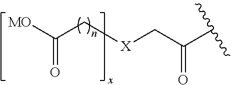

n=1−4, x=1−3, X=N, O, S, such as:

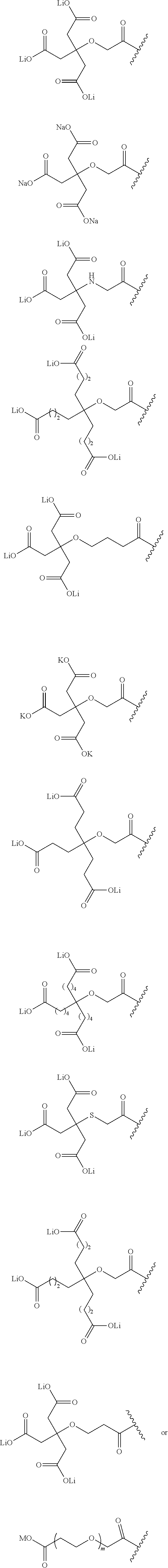

m=1−5, such as

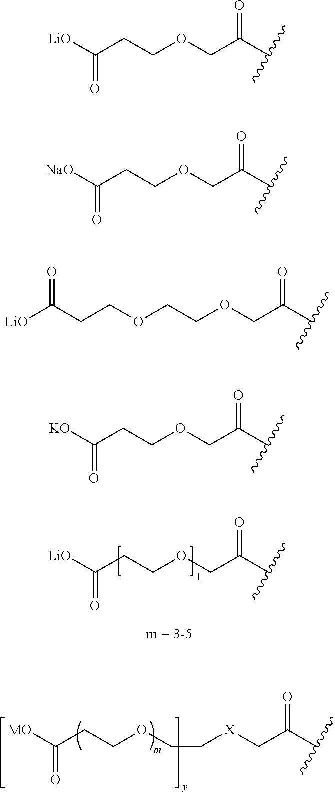

m=1−5, y=2−3, X=N, O, S, such as:

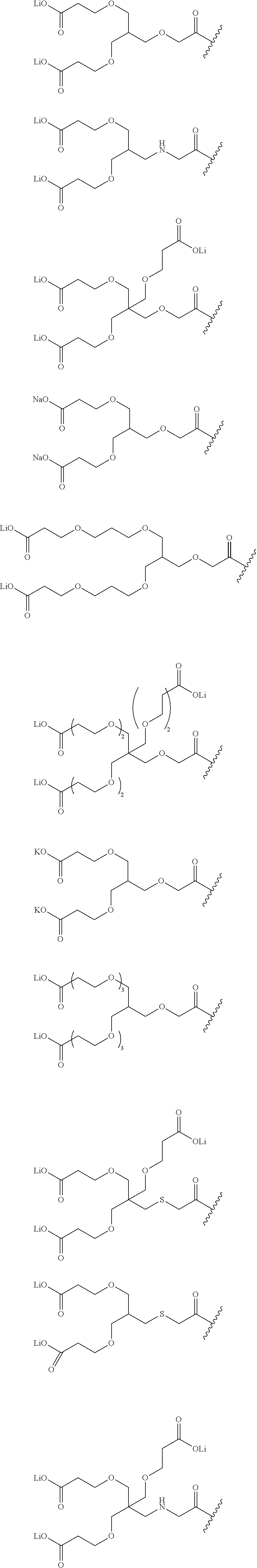

6. The composition of claim 1, wherein R1 comprises:

C3-C6 cycloalkyl or cycloheteroalkyl, such as: cyclohexyl.

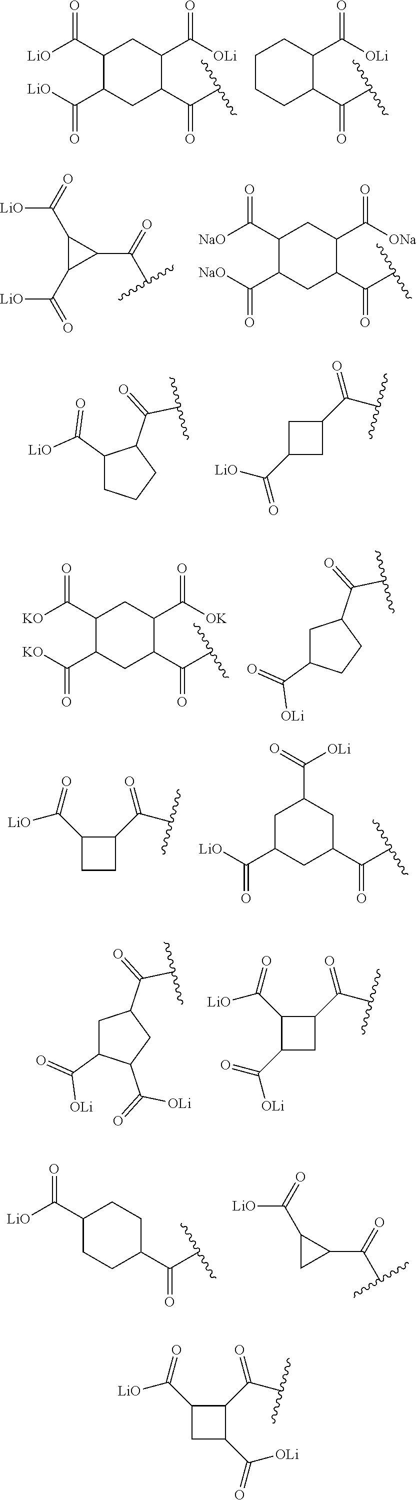

7. The composition of claim 1, wherein R1 comprises:

C5-C6 aryl or heteroaryl, such as:

phenyl, pyridinyl, diazine (e.g. 2, 3 or 4-pyrindinyl, 3,5 or 3,6-diazinyl)

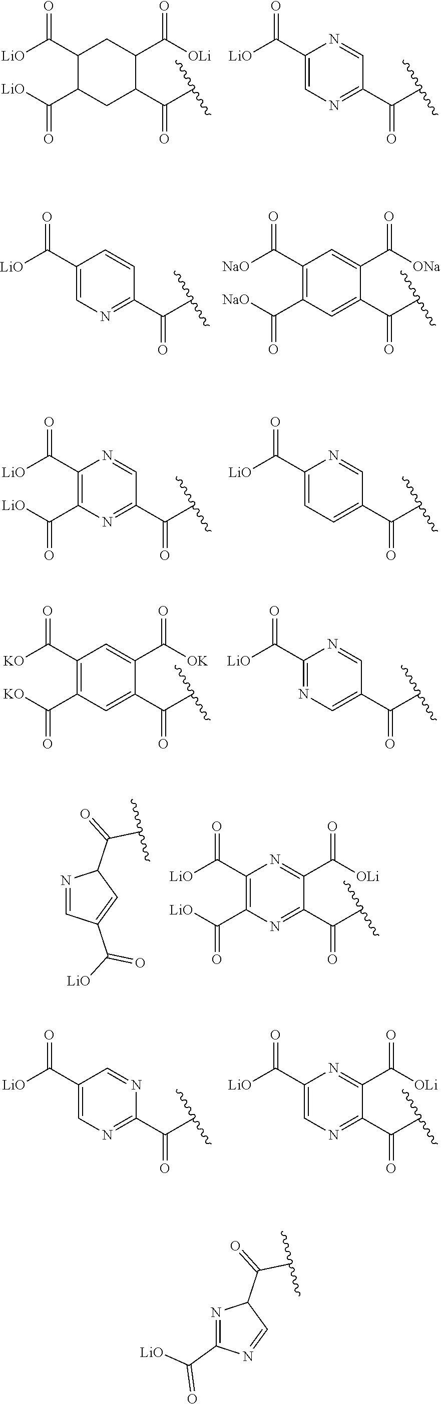

8. The composition of claim 1, wherein R1 comprises:

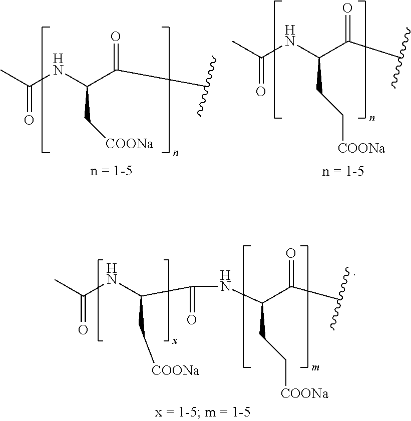

Two types of amino acids can be mixed in the sequence.

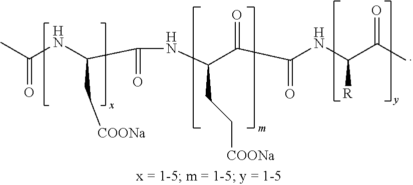

R stands for amino acid residue. Three types of amino acids can be mixed in the sequence.
9. The composition of claim 1, wherein R1 comprises:

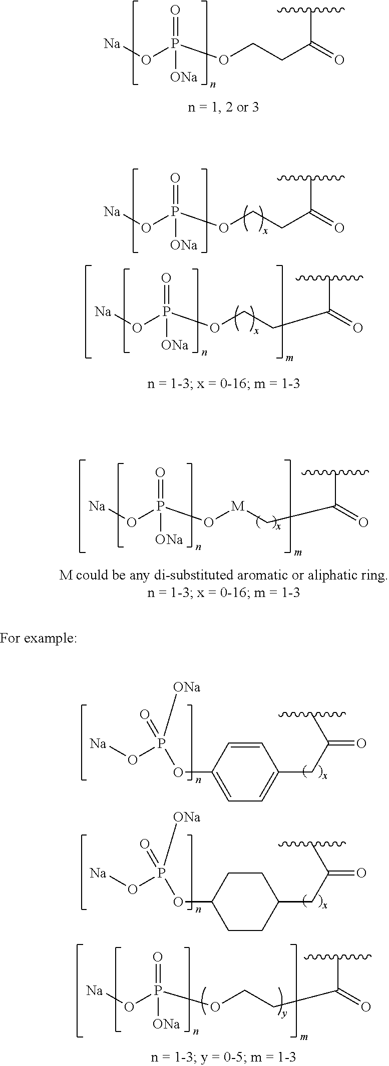

10. The composition of claim 1, wherein R1 comprises:

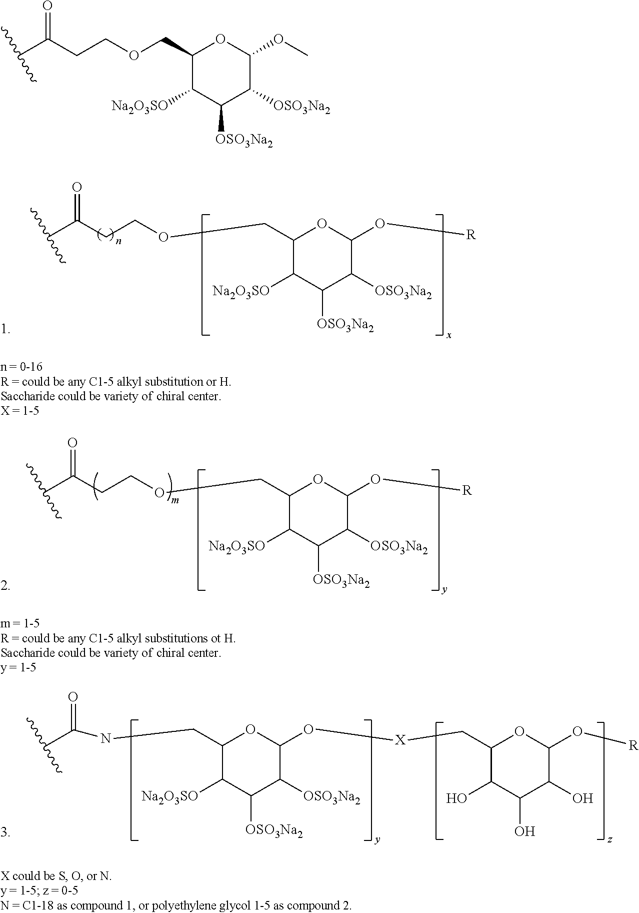

11. A composition of any of claims 1-10, wherein:

R2 comprises steroid selected from:

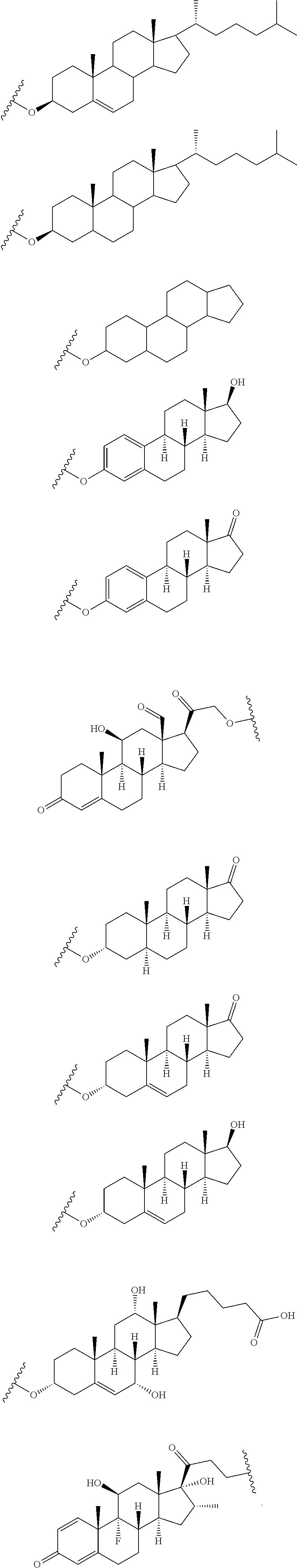

12. A composition of any of claims 1-10, wherein R2 comprises an alkyl chain:

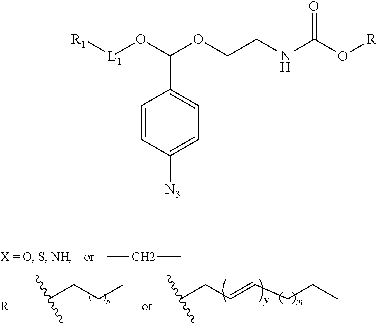

n=4−16; m=1−13; y=1−3, wherein the olefin(s) can be Z/E, and in any position(s) of the chain, such as:

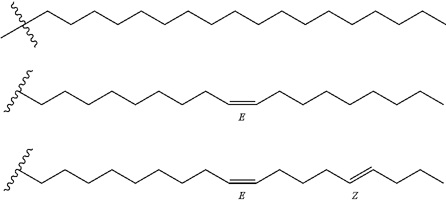 [SMILES: *CCCCCCCCCCCCCCCCCC ; *CCCCCCCC/C=C\CCCCCCCC ; *CCCCCCCC/C=C\CCC/C=C/CCC]

13. A composition of any of claims 1-10, wherein R2 comprises an alkyl chain, of structure:

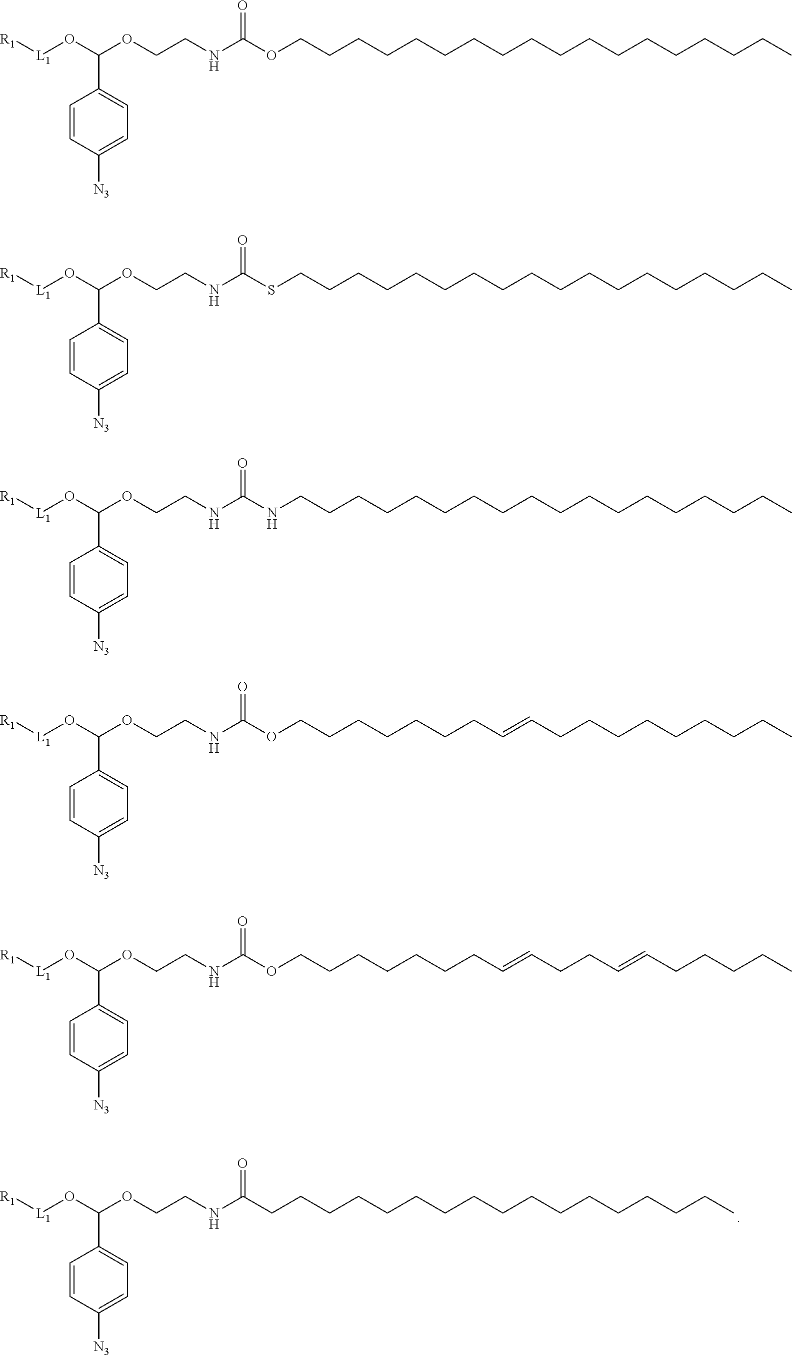

14. A composition of any of claims 1-10, wherein R2 comprises two alkyl chains:

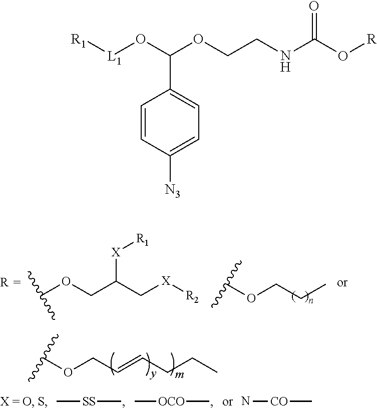

n=4−16; m=1−13; y=1−3, wherein the olefin(s) can be Z/E, and in any position(s) of the chain, such as:

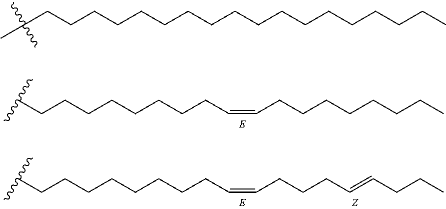 [SMILES: *CCCCCCCCCCCCCCCCCC ; *CCCCCCCC/C=C\CCCCCCCC ; *CCCCCCCC/C=C\CCC/C=C/CCC]

15. A composition of any of claims 1-10, wherein R2 comprises two alkyl chains, of structure:

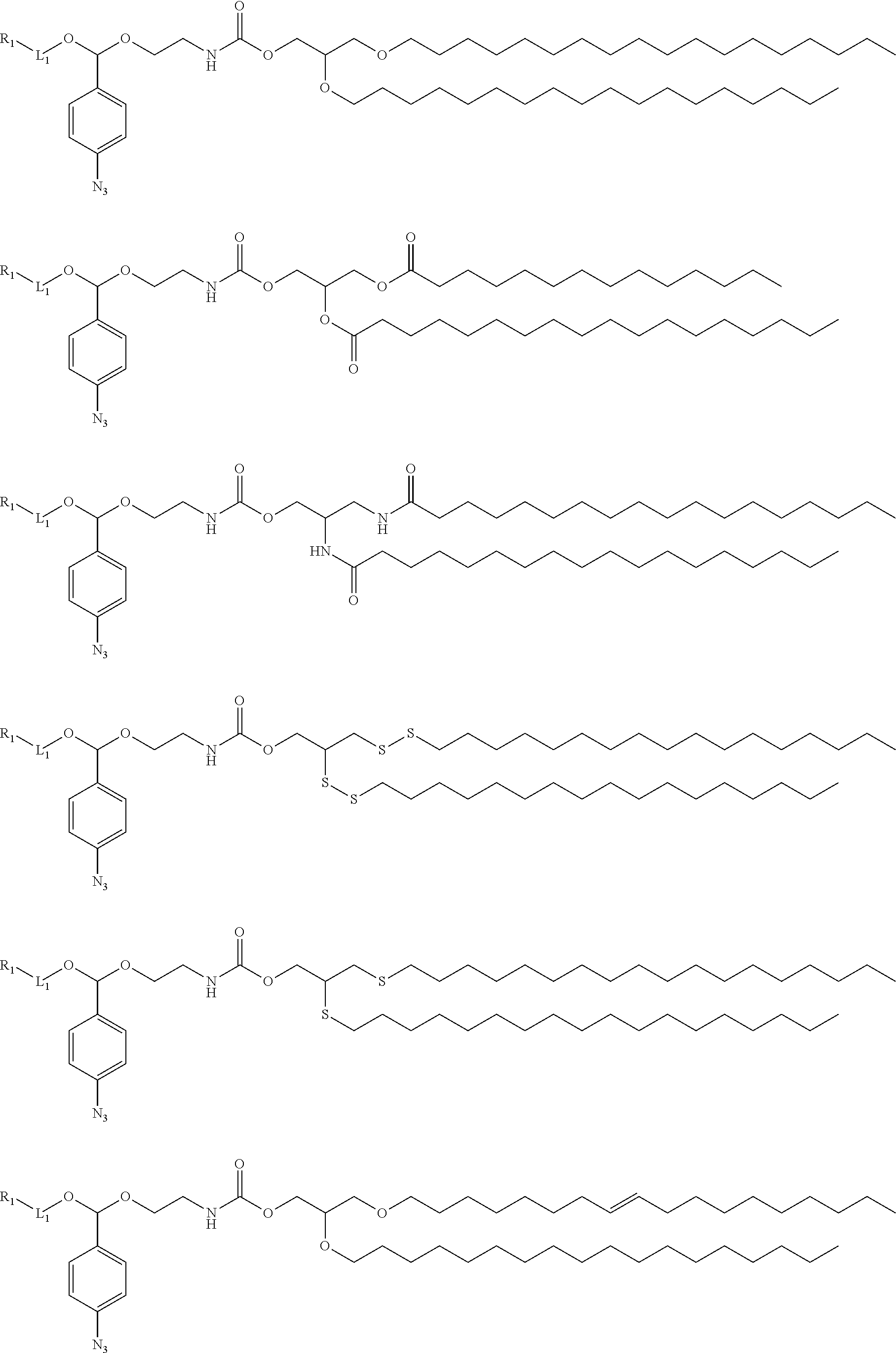

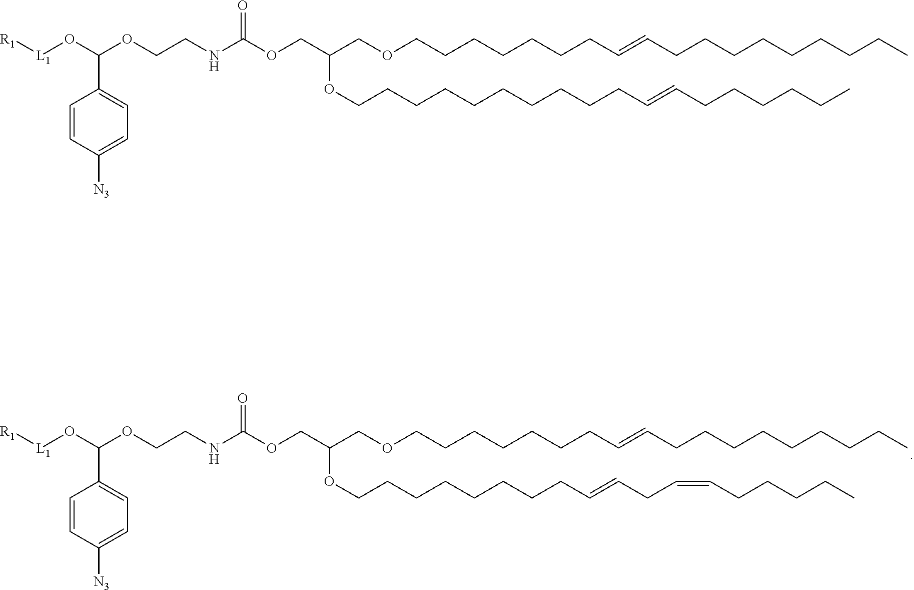

16. A composition of any of claims 1-10, wherein R2 comprises three alkyl chains:

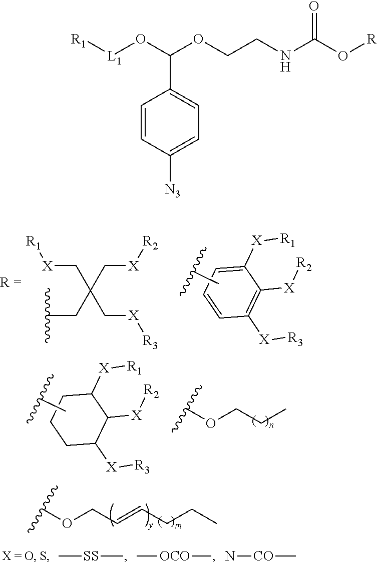

n=4−16; m=1−13; y=1−3, wherein the olefin(s) can be ZIE, and in any position(s) of the chain, such as:

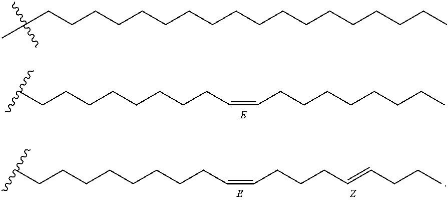 [SMILES: *CCCCCCCCCCCCCCCCCC ; *CCCCCCCC/C=C\CCCCCCCC ; *CCCCCCCC/C=C\CCC/C=C/CCC]

17. A composition of any of claims 1-10, wherein R2 comprises three alkyl chains, of structure:

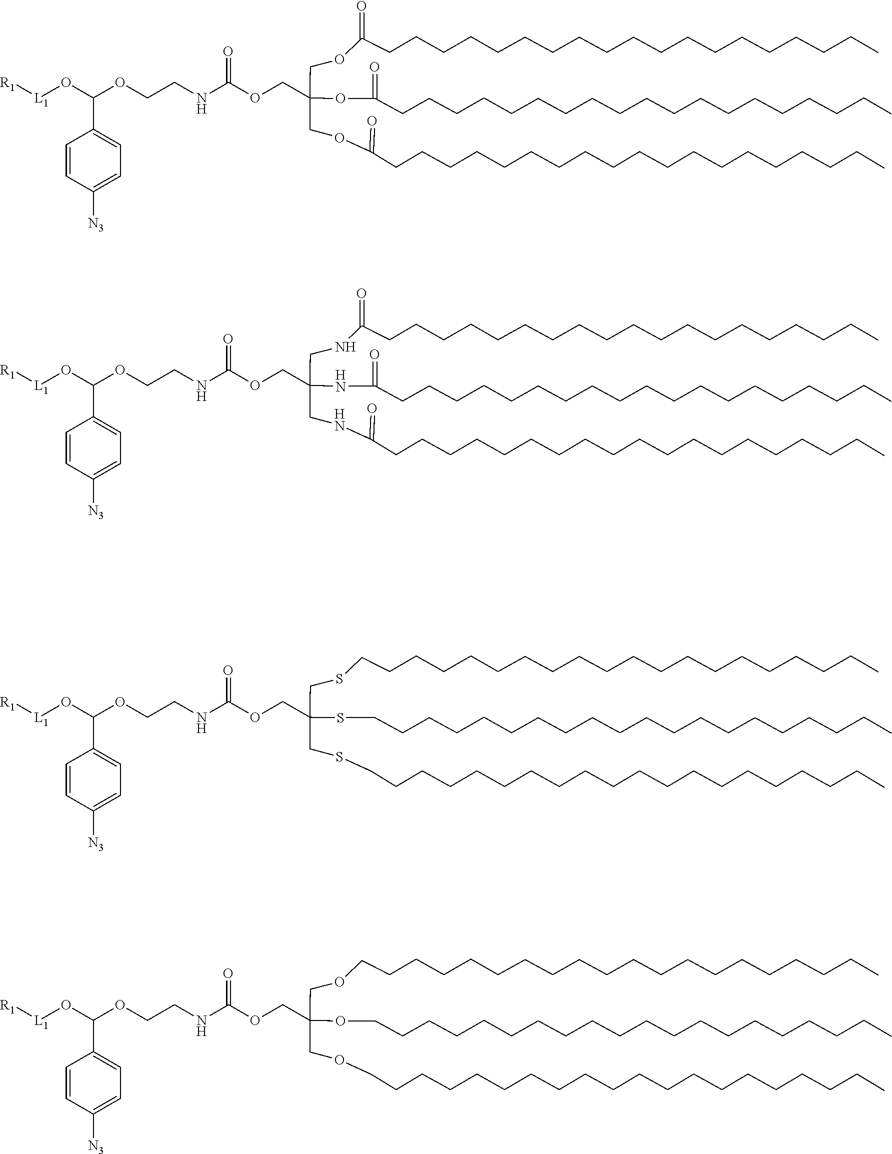

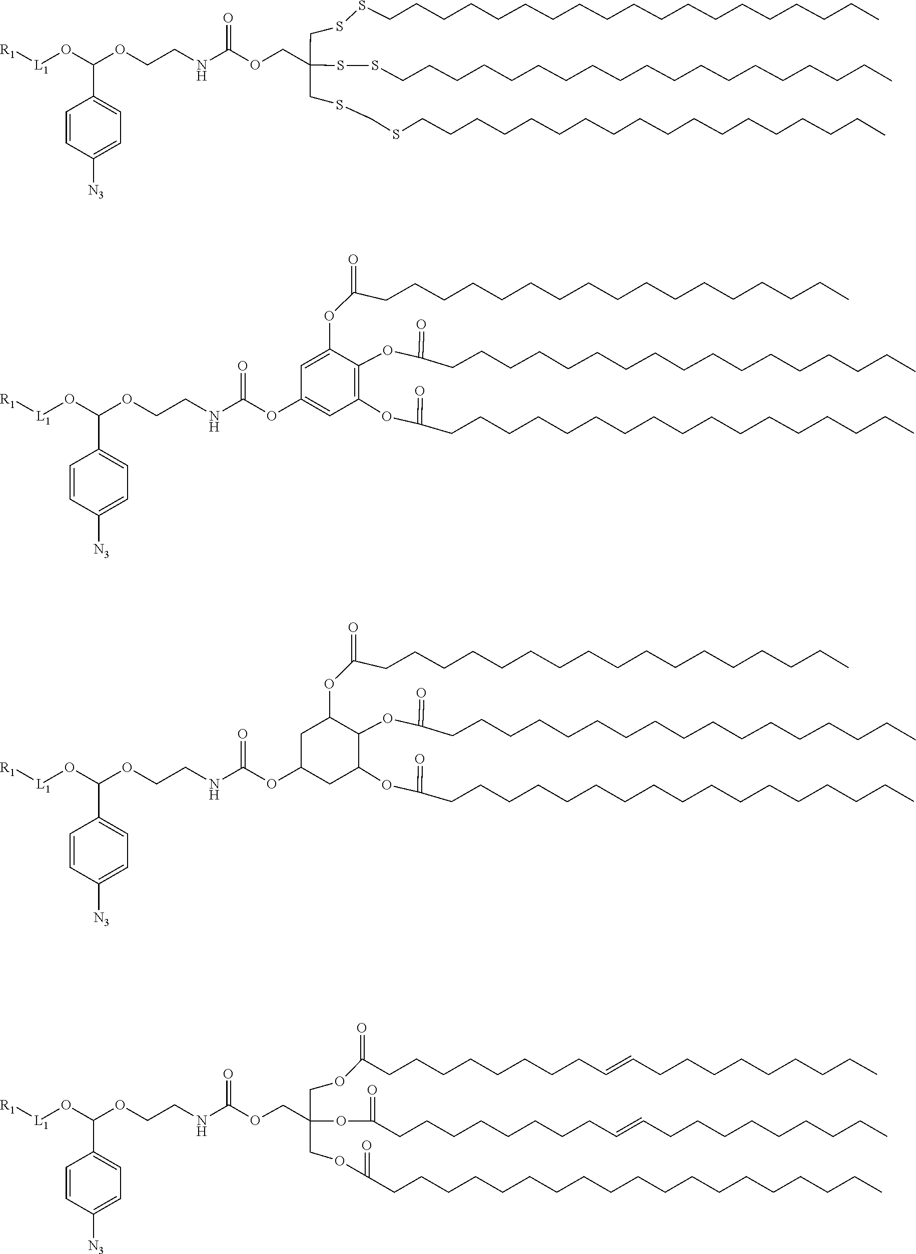

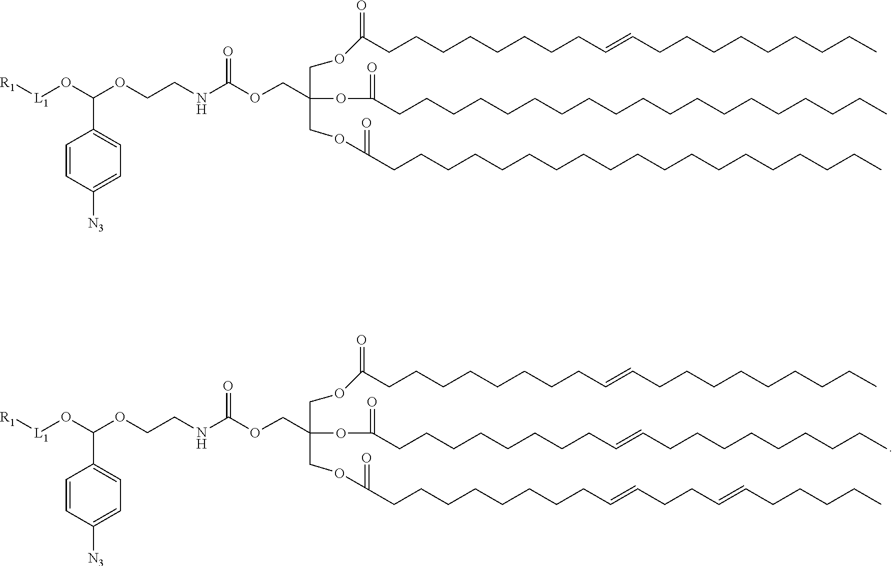

18. A composition of any of claims 1-10, wherein:

L1, L2 and L3 comprise linkers independently optionally hetero-, optionally substituted linear C1-C12 alkyl; or

L1, L2 and L3 comprise linkers independently selected from:

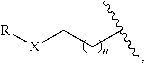

n=1−5; X=N, O, S; or

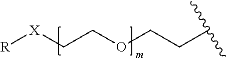

m=0-5, X=N, O, S; or

L1 and L2 are —CH2CH2NH-R1 and —CH2CH2NHCO-R2.
19. A composition of any of claims 1-10, wherein the azide is reduced to an amine (e.g. prior to injection, for rapid hydrolysis).
20. A lipid nanoparticle (LNP) composition comprising a compound of any of claims 1-10, configured, for example, to deliver mRNA, plasma DNA, siRNA, for example, for vaccines, wherein for some of the mRNA, like Cas 9 mRNA may be combined with guide RNA, for cell editing and treating disease.
21. A composition of any of claims 1-10, formulated into lipid nanoparticles (LNPs) further comprising a nucleic acid, such as an RNA or DNA, encoding a therapeutic protein, vaccine antigen, or gene editing enzyme(s).
22. A method of using a compound of any of claims 1-10 comprising delivering the compounds in a lipid nanoparticle (LNP) composition comprising a compound herein, to transfect a tissue or organ, such as muscle, lung, spleen, liver and blood.
23. A method of using a compound of any of claims 1-10 comprising delivering the compounds in a lipid nanoparticle (LNP) composition comprising a compound herein configured as a vaccine or therapeutic, and preferably detecting a resultant intended, targeted effect, and preferably with enhanced effect, e.g. mRNA transfection efficiency attributable to use of the compound.
24. A method of making a compound of any of claims 1-10, comprising solid phase peptide synthesis.
25. A solid-phase lipid synthesis method comprising synthesis of cationic, ioniable lipids via solid phase peptide synthesis.
26. The method of claim 25 comprising integration in an automated robotic system (ARS) of: (i) the solid phase lipid synthesis, (ii) initial cell screening, and (iii) animal organ or cell targeting.
27. A compound, composition or method as described herein.

The invention encompasses all combinations of the particular embodiments recited herein, as if each combination had been laboriously recited.

### BRIEF DESCRIPTION OF THE DRAWINGS

FIGS. 1a-d. Rapidly degrading lipid nanoparticles (RD-LNPs) enhance the delivery of mRNA in multiple organs in vivo. a, New acid-degradable linker, termed an azido-acetal, which hydrolyzes with a half-life of 21 days at pH 7.4 and also rapidly hydrolyzes in endosomes and has a pH 6.0 hydrolysis half-life of 14 minutes. The azido-acetal was used to synthesize new acid-degradable lipids and lipid nanoparticles (RD-LNPs), described in b-d. Prior to administration, the azido in the azido-acetal linker is reduced to an amine with DTT and this accelerates its hydrolysis rate by a factor of 255. Three new acid-degradable lipids were synthesized with the azido-acetal linker (described in b-d), and LNPs made with these new lipids (RD-LNPs) were superior to traditional LNPs in multiple ways. b, LNPs containing 1 (ADP-LNPs) maintain high PEGylation extracellularly, but rapidly release their PEGs post-endocytosis and this promotes fusion with endosomes. ADP-LNPs enhanced the delivery of mRNA to the liver and spleen, and also transfected brain tissue efficiently, due to their enhanced diffusion from the injection site. c, LNPs containing 2 (ADA-LNPs) have a high negative charge density and transfect the spleen and liver efficiently. ADA-LNPs hydrolyze in endosomes and this facilitates endosomal disruption via osmotic destabilization of the endosome and by increasing the positive charge density of ADA-LNPs. d, LNPs containing 3 (ADC-LNPs) have a high positive charge density, resulting in efficient lung targeting. ADC-LNPs hydrolyze in endosomes and cause rapid release of mRNA and also endosomal disruption via the colloid osmotic effect, due to decomposition of the cationic lipid.

FIGS. 2a-j. ADP (1) enhances the transfection efficiency of LNPs in vitro and in vivo. a, LNPs made with 1 (ADP-LNPs) rapidly release their PEG chains in endosomes and trigger endosomal disruption. ADP-LNPs also tolerate high levels of PEGylation and this enhances transfection after a direct tissue injection and increases their circulation half-life. In contrast, LNPs made with PEG2k-Cholesterol (NDP-LNPs) retain their PEG in endosomes and have low levels of endosomal disruption. Scale bar in the Cryo-TEM figure is 25 nm. b, LNPs were formulated with 1 (ADP-LNPs) and compared against LNPs with traditional PEG-lipids (NDP-LNPs). Transfection of HEK 293T cells with EGFP mRNA encapsulated in ADP-LNPs or NDP-LNPs. ADP-LNPs and NDP-LNPs were made with different percentages of 1 or PEG2k-cholesterol respectively. After 24 hours of incubation, cells were analyzed for transfection efficiency via flow cytometry; mRNA=3 μg/mL, n=3. c, Transfection of HEK 293T cells with luciferase mRNA encapsulated by ADP-LNPs or NDP-LNPs. Quantification of luciferase expression in HEK 293T cells was determined with a luciferase kit after 24 hours of incubation; mRNA=3 μg/mL, n=3. d, ADP-LNP/EGFP mRNA complexes transfect 90% of HSPCs (right side) and induced only 41% cell toxicity (left side). In contrast, the DLin-MC3-DMA standard LNP (Std-LNP) caused 95% cell toxicity and transfected only 50% of the remaining cells, and electroporation (EP) of cells with EGFP mRNA killed more than 65% of cells and transfected about 82% of the remaining cells. e, HSPCs were transfected with ADP-LNPs (10 mole % 1) with Cas9 mRNA and gRNA targeting the AAVS1 gene. Indels were analyzed by Sanger sequencing after 48 hours; mRNA=20 μg/mL, n=3. f, Mice were transfected with ADP-LNPs (10 mole % 1) or NDP-LNPs (10 mole % PEG2k-cholesterol) containing luciferase mRNA. 4 hours after injection, organs were extracted for luciferase activity analysis. mRNA=0.5 mg/kg, n=3. g, ADP-LNPs efficiently transfect the liver. ADP-LNPs (10 mole % 1) containing Cre mRNA or saline were intravenously injected into Ai9 mice. After 2 weeks, the organs were harvested and analyzed via flow cytometry for tdTomato fluorescence and h, histological sections; mRNA=5 mg/kg, n=3. i, ADP-LNPs deliver Cas9 mRNA and gRNA to brain tissue more efficiently than Std-LNPs. ADP-LNPs (10 mole % 1) containing Cas9 mRNA/Ai9 sgRNA (0. 225 μg/μL Cas9 mRNA) were injected intracranially and compared against Std-LNPs. Quantification of the number of transfected cells (bar graph) 21 days after injection, n=4. j, A representative histological section is shown on the right side; Scale bar, 30 um.

FIGS. 3a-i. ADA (2) efficiently disrupts endosomes and enhances the transfection efficiency of LNPs in cells and in vivo. a, ADA-LNPs are negatively charged in the blood, but after endocytosis increase their positive charge density and increase the osmolarity of the endosome, resulting in enhanced endosomal disruption. In contrast, LNPs made with 18PA or 6 (NDA₁-LNPs or NDA2-LNPs) have low transfection efficiency because of their permanent negative charges. The scale bar in the Cryo-TEM figure is 50 nm. b, Transfection of HEK 293T cells with EGFP mRNA encapsulated by ADA-LNP or NDA₁-LNPs. ADA-LNPs and NDA₁-LNPs were made with different percentages of 2 or 18PA respectively and added to cells. After 24 hours of incubation, the cells were analyzed via flow cytometry; mRNA=3 μg/mL, n=3. c, Transfection of HEK 293T cells with luciferase mRNA encapsulated in ADA-LNPs or NDA₁-LNPs. Quantification of luciferase expression in HEK 293T cells was determined with a luciferase kit after 24 hours of incubation; mRNA=3 μg/mL, n=3. d, ADA-LNPs (16 mole % 2) or NDA₁-LNPs (48 mole % 18PA) containing IL-22 mRNA were delivered to HEK 293T cells and after 24 hours, the amount of IL-22 protein was determined via ELISA; mRNA=1 μg/mL, n=3. e, Quantum dots (QDs) conjugated to DNA were encapsulated in St-LNPs, ADA-LNPs and NDA₁-LNPs and the level of endosomal disruption was determined by measuring their intracellular trajectories. The fraction of QDs released from the endosome (mobile fraction) for each group was gated as the proportion of trajectories shorter than 50 frames with diffusion coefficient greater than 0.1 μm²/s. QDs in ADA-LNP have the highest mobile fraction of the three LNP groups, indicating efficient endosomal release. The negative control was QDs immobilized on glass (immovable) and the positive control was QDs delivered into the cytoplasm via osmotic shock. f, ADA-LNPs (16 mole % 2), NDA₁-LNPs (48 mole % 18PA), or NDA2-LNPs (16 mole % 6) containing luciferase mRNA were administered to mice, and after 4 hours luciferase activity in the spleen was measured; mRNA=0.5 mg/kg, n=3. g, ADA-LNPs (16 mole % 2) containing Cre mRNA and saline were injected into Ai9 mice via the retro-orbital vein, and after 2 weeks, the organs were analyzed via flow cytometry for tdTomato fluorescence and in h, histological sections. i, Immunohistochemistry analysis of ADA-LNP treated mice showed transfection of macrophages (F4/80+stain) and B-cells (B220⁺ stain) in the spleen, and hepatocytes in the liver; mRNA=5.0 mg/kg, n=3.

FIGS. 4a-l. ADC (3) degrades into biocompatible products and enhances the transfection efficiency of LNPs in cells and in vivo via multiple mechanisms. a, ADC-LNPs have a high positive charge density and target LNPs to the lung. In addition, ADC-LNPs degrade into non-toxic fragments and trigger endosomal disruption by increasing the osmolarity of the endosome. In contrast, NDC-LNPs degrade into membrane impermeable cations, which accumulate in cells and prevent chronic administration. Scale bar in the Cryo-TEM figure is 50 nm. b, Transfection of HEK 293T cells with EGFP mRNA encapsulated in ADC-LNPs or NDC-LNPs. ADC-LNPs and NDC-LNPs were made with different percentages of 3, or DOTAP respectively. After 24 hours of incubation, cells were analyzed for transfection efficiency via flow cytometry; mRNA=3 μg/mL, n=3. c, Transfection of HEK 293T cells with luciferase mRNA encapsulated in ADC-LNPs or NDC-LNPs. Quantification of luciferase expression in HEK 293T cells was determined with a luciferase kit after 24 hours of incubation; mRNA=3 μg/mL, n=3. d, ADC-LNPs (40 mole % 3) and NDC-LNPs (40 mole % DOTAP) containing IL-22 mRNA were delivered to HEK 293T cells and the amount of IL-22 protein was determined via ELISA at various time points; mRNA=1 μg/mL, n=3. e, ADC-LNPs transfected human HSPCs with high efficiency and low toxicity. ADC-LNPs containing 5 or 10mole % of 3 transfected 70% of HSPCs (right side) and had more than 90% cell viability (left side) at a dose of 20 μg/mL of EGFP mRNA. f, ADC-LNPs delivered Cas9 mRNA and gRNA to HSPCs and edited the AAVS1 gene efficiently. ADC-LNPs (10 mole % 3) containing Cas9 mRNA and gRNA induced approximately 50% indels in HSPCs after 48 hours of incubation; mRNA=20 μg/mL, n=3. g, Mice were treated with luciferase mRNA encapsulated in ADC-LNPs (40 mole % 3) or NDC-LNPs (40 mole % DOTAP). After 4 hours, the organs were analyzed for luciferase activity using a luciferase kit; mRNA=0.5 mg/kg, n=3. h, ADC-LNPs (40 mole % 3) containing Cre mRNA or saline were injected intravenously into Ai9 mice via the retro-orbital route, and after 2 weeks, the organs were analyzed via flow cytometry for tdTomato fluorescence and I, histology sections; mRNA=5.0 mg/kg, n=3. j, Histological analysis of ADC-LNP treated mice demonstrates that epithelial cells (E-cad⁺stain) and endothelial cells (CD31⁺ stain) in the alveolus are efficiently transfected. k, ADC-LNPs (40 mole % 3) containing IL-22 mRNA were injected into mice and the amount of IL-22 protein in the blood was determined via ELISA at various time points. ADC-LNPs reached a peak concentration of 120 ng/ml and at 24 hours was 20 ng/mL, which is still above the threshold for IL-22 signaling; mRNA=2.5 mg/kg, n=3. 1, ADC-LNPs can improve the treatment of acute lung injury. Mice were injected intratracheally with LPS (5 mg/kg) and given an intravenous injection of ADC-LNPs or Std-LNPs containing IL-22 mRNA. 4 days post injection, the mice were sacrificed and samples of bronchoalveolar lavage fluid (BALF) were collected for protein content in the lung. ADC-LNPs containing IL-22 mRNA (mRNA=2.5 mg/kg, n=6) significantly reduced the protein content in the lungs of LPS-treated mice, whereas Std-LNPs could not.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### Proof of Concept: Lipid Nanoparticles with Acid Degradable PEG-Lipids Transfect mRNA Efficiently Tissue with Specificity

Lipid nanoparticles with representative acid degradable PEG-lipids (500 DA, 1 kD, and 2kD PEG) transfect mRNA in HELA and HEK cells efficiently, and transfect muscle tissue efficiently, and transfect systemically in mice after intravenous injection, transfect cells in the blood, transfect mRNA into brain tissue after an intracranial injection, transfect lung tissue with specificity; and transfect spleen tissue with specificity after an intravenous injection.

Exemplary experimental Protocols. LNPs made with representative compounds deliver luciferase mRNA and CRE mRNA to the lungs with specificity. LNPs containing various mole ratios of compounds are screened in mice for their ability to deliver luciferase mRNA, after an intravenous injection (10 ug mRNA per mouse). These results are further validated via separate CRE mRNA delivery experiments using Ai9 mice. Ai9 mice are given three consecutive injections of CRE mRNA (0.5 mg/kg per dose, 2 days apart) at 10 ug mRNA per dose, and two days after the last injection the mice are sacrificed, and the lung tissue is analyzed for fluorescence and compared against saline treated Ai9 mice. Ai9 mice treated with LNPs containing representative compounds had numerous red cells in their lung histology sections. These results indicate that LNPs made with these compounds can deliver ASOs and CFTR mRNA to lung tissue.

### Examples: New Acid Degradable Lipids Based on Self Assembling Peptides

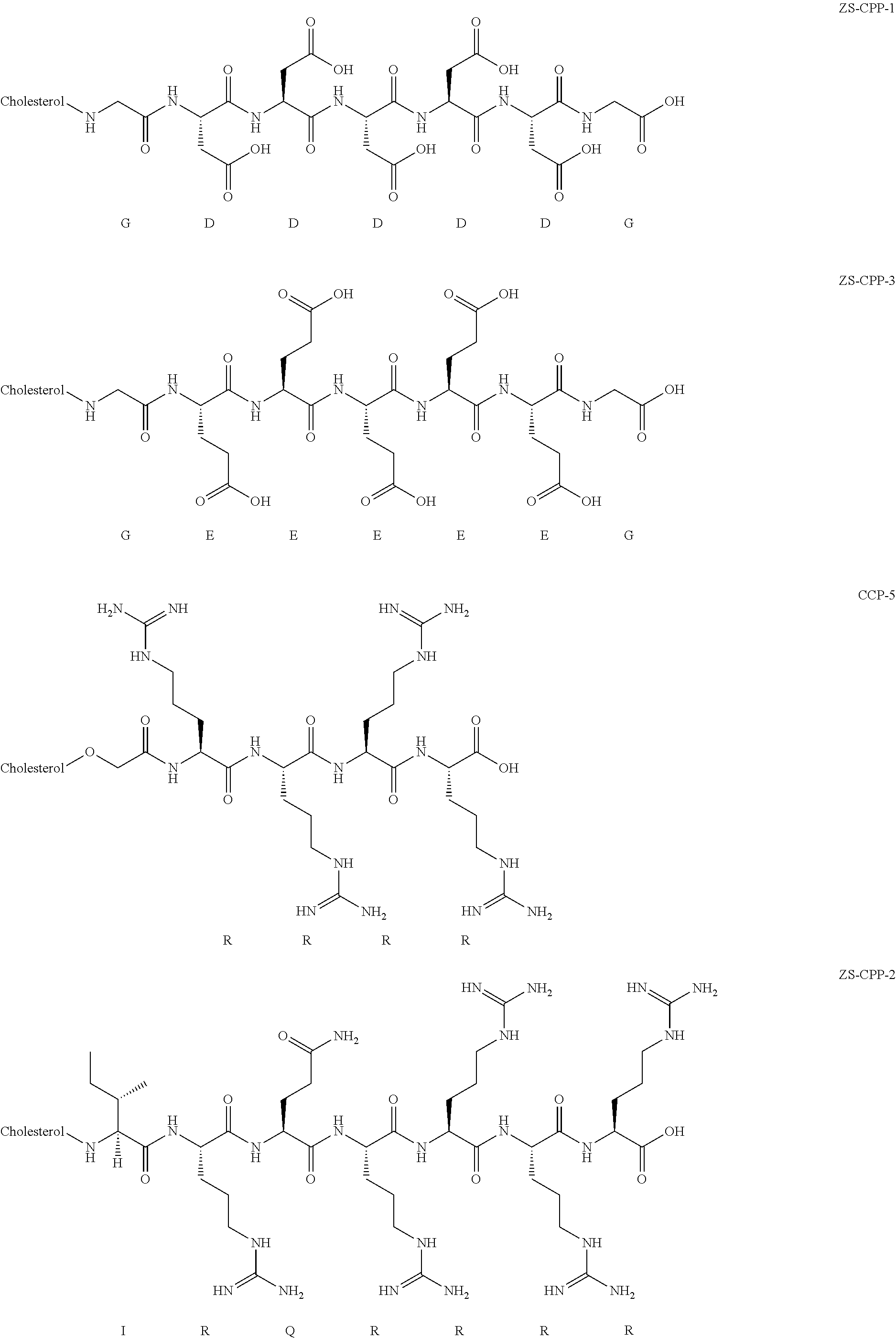

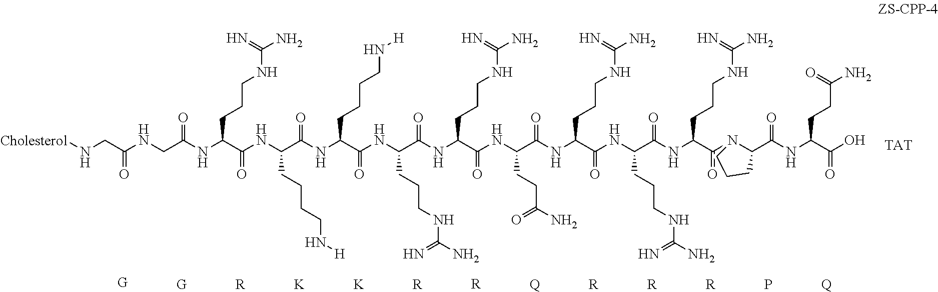

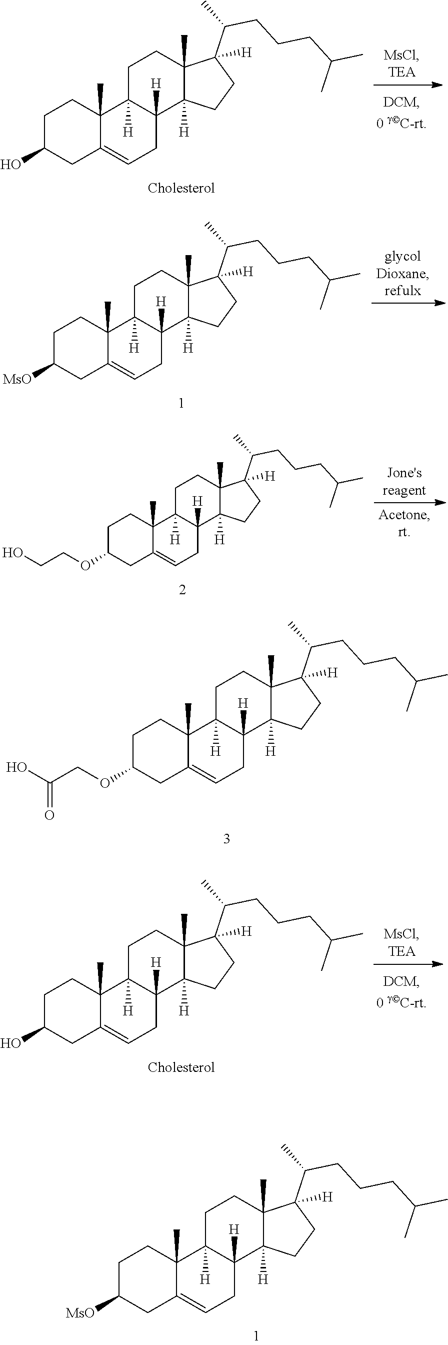

Cholesterol (20 g, 51.73 mmol, 1.0 equiv) was added to a round bottom flask with 40 0mL at room temperature, followed by adding triethylamine (14.35 mL, 103.5 mmol, 2 equiv). Then methane sulfonyl chloride (4.8 mL, 62.1 mmol, 1.2 euiqv) was added dropwise in ice bath under vigorously stirring. Monitor the reaction by TLC, generally the reaction will complete 30 min. When completed, the reaction was washed by 0.5 M HCl (100 mL×3), saturated aqueous NaHCO₃ (50 mL×3) and NaCl brine (100 mL×2), dried the organic phase by MgSO4, then removal of the solvent under reduced pressure. The concentrated residue was recrystallized by diethyl ether. The crude product (yield quantitative, off-white powder) was used in the next step without further purification.

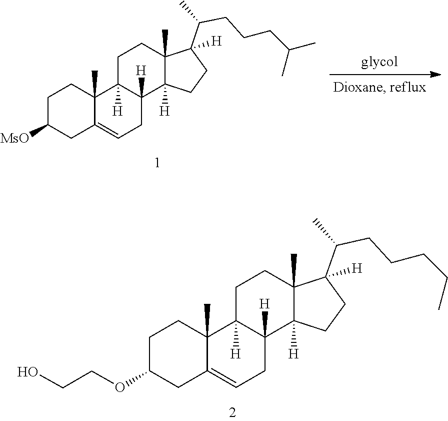

To a solution of cholesteryl mesylate (51.73 mmol) in dry 1,4-dioxane (350 mL), glycol (85 mL, 1.552 mol, 30 equiv) was added and refluxed for about 2 hours until complete disappearance of the starting material (monitored by TLC). After concentration of the reaction mixture, the residue was dissolved in 200 mL of DCM. Solution was washed with saturated aqueous NaHCO₃ (100 mL×3), water (100 mL×3) and NaCl brine (100 mL×2). The organic phase was dried with sodium MgSO₄, solvent was removed under reduced pressure. The concentrated residue was recrystallized by DCM and hexane. The crude product (17.4 g, 78%, off-white powder) was used in the next step without further purification.

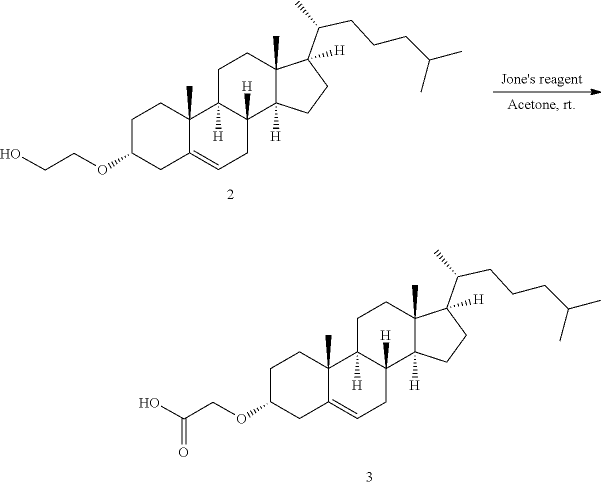

To a solution of cholesteryl glycol (17 g, 39.47 mmol, 1.0 equiv) in 500 mL of acetone, 2M Jones reagent (32 mL, 63.15 mmol, 1.6 equiv) was added while stirring. The mixture was monitored by TLC. 1 hour later, additional 2M Jones reagent (8 mL, 15.78 mmol, 0.4 equiv) was added and the reaction was kept stirring at room temperature over 3 hours. Then green precipitate was filtered off and solvent evaporated to dryness. Residue was dissolved in DCM, washed with saturated sodium NaCl brine (100 mL x 2). and dried with sodium MgSO4. Then it was filtered through Celite and evaporated to dryness. The partial of the product can be purified by recrystallization (dichloride methane and hexane, 8.5 g, 37%), product in the filtrate can be purified by the column chromatography on silica gel (DCM/MeOH gradient 0-15%). Yields 6.4 g (28%)

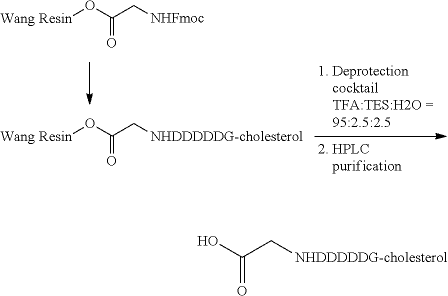

Typical synthetic procedure (for CCP1): Wang Resin (150 mg, 0.0585 mmol, 0.39 mmol/g) was added to the reactor. Concentration of synthons:

| Amino acids | Cholesterol acid | HBTU | Piperidine |
|---|---|---|---|
| 0.4M | 0.25M | 0.4M | 0.8M |

### Solvent is DMF

The peptide synthesis was performed on an Aapptech Eclipse solid phase peptide synthesizer, using standard FMOC synthesis procedures, following the manufacturers recommended procedure. The cholesterol acid was added as the last amino acid at 0.25 Molar concentration, on the solid phase.

| | | |
|---|---|---|
| Components | MW | Volume/μL (mole %) |

Cpp1 formula

| | | | | | | | | |
|---|---|---|---|---|---|---|---|---|
| ZS-CPP-1 | 1134.2 | 0.25 (5%) | 0.54 | (10%) | 1.2 | (20%) | 3.2 | (40%) |

| | | | | | |
|---|---|---|---|---|---|
| PEG2K-DMG | 2500 | 0.5 | 0.5 | 0.5 | 0.5 |
| DOPE | 744.3 | 3 | 3 | 3 | 3 |
| D-Lin | 642.09 | 4 | 4 | 4 | 4 |
| Chol | 386.64 | 2.5 | 2.5 | 2.5 | 2.5 |

Cpp2 formula

| | | | | | | | | |
|---|---|---|---|---|---|---|---|---|
| ZS-CPP-2 | 1466.93 | 0.33 (5%) | 0.69 | (10%) | 1.56 | (20%) | 4.15 | (40%) |

| | | | | | |
|---|---|---|---|---|---|
| PEG2K-DMG | 2500 | 0.5 | 0.5 | 0.5 | 0.5 |
| DOPE | 744.3 | 3 | 3 | 3 | 3 |
| D-Lin | 642.09 | 4 | 4 | 4 | 4 |
| Chol | 386.64 | 2.5 | 2.5 | 2.5 | 2.5 |

Cpp3 formula

| | | | | | | | | |
|---|---|---|---|---|---|---|---|---|
| ZS-CPP-3 | 1204.38 | 0.27 (5%) | 0.57 | (10%) | 1.28 | (20%) | 3.4 | (40%) |

| | | | | | |
|---|---|---|---|---|---|
| PEG2K-DMG | 2500 | 0.5 | 0.5 | 0.5 | 0.5 |
| DOPE | 744.3 | 3 | 3 | 3 | 3 |
| D-Lin | 642.09 | 4 | 4 | 4 | 4 |
| Chol | 386.64 | 2.5 | 2.5 | 2.5 | 2.5 |

Cpp4 formula

| | | | | | | | | |
|---|---|---|---|---|---|---|---|---|
| ZS-CPP-4 | 2105.67 | 0.47 (5%) | 1.0 | (10%) | 2.23 | (20%) | 5.95 | (40%) |

| | | | | | |
|---|---|---|---|---|---|
| PEG2K-DMG | 2500 | 0.5 | 0.5 | 0.5 | 0.5 |
| DOPE | 744.3 | 3 | 3 | 3 | 3 |
| D-Lin | 642.09 | 4 | 4 | 4 | 4 |
| Chol | 386.64 | 2.5 | 2.5 | 2.5 | 2.5 |

Cpp5 formula

| | | | | | | | | |
|---|---|---|---|---|---|---|---|---|
| ZS-CPP-5 | 1068 | 0.14 (3%) | 0.62 | (12%) | 1.0 | (18%) | 1.35 | (23%) |

| | | | | | |
|---|---|---|---|---|---|
| PEG2K-DMG | 2500 | 0.5 | 0.5 | 0.5 | 0.5 |
| DOPE | 744.3 | 3 | 3 | 3 | 3 |
| D-Lin | 642.09 | 4 | 4 | 4 | 4 |
| Chol | 386.64 | 2.5 | 2.5 | 2.5 | 2.5 |

Concentration of PEG2K-DMG, DOPE,D-Lin, choleterol, DOTAP are 10 mg/mL in ethanol. The concentration of All choleterol-peptides are 40 mg/mL in DMSO

For LNP preparation a stock solution (10 mg mL⁻¹ ) of D-Lin, DOPE, Cholesterol, DMG-PEG and was made by dissolving cach lipid separately in ethanol and all cholesterol-peptide lipids were 40 mg mL⁻¹ in DMSO stored at −30° C. Just before the LNP formation, lipids were taken out and kept on ice and vortexed whenever necessary. The cholesterol solution was slightly warmed up to dissolve the crystals which form during the cold storage. Then D-Lin, DOPE, Cholesterol, DMG PEG and cholesterol-peptide lipids were mixed in variety molar ratios listed in table 1. RNAs 1 μg μL⁻¹ (Luciferasc-mRNA, cGFP mRNA) were mixed with LNP and PBS (pH 7.2) in a 1:1:2 ratio (vol/vol). The resulting LNPs were gently vortexed and incubated at room temperature for 15 minutes for immediate use or stored at 4° C.

LNP in vivo: BALB/c mice with age of 16-24 weeks were i.v. (retro-orbital) injected with various luciferase mRNA LNP formulations. LNPs were formed as described in table 1 and mice were i.v. (retro-orbital) injected with a dose of 10 μg Luc mRNA per mouse. After 5 h, mice were injected with d-Luciferin (150 mg kg⁻¹, intraperitoneal) and imaged using an IVIS Lumina system (Perkin Elmer)

LNP in vitro: HEK 293T cells were seeded in 96 well plate at 105 cells mL⁻¹ and cultured with DMEM medium (10% FBS and 1% P.S) under 5% CO₂ at 37° C. for 18-24 h before transfection. LNPs were formed as described in table 1 and 300 ng cGFP mRNA was added to each well with 100 μL Opti serum free medium. GFP-positive cells were quantified by FACS (Attune) analysis 24 after treatment.

Jurkat cell were diluted to 2×10⁵ cells mL⁻¹ and cultured with RPMI1640 medium (10% FBS and 1% P.S) under 5% CO₂ at 37° C. LNPs were formed as described in table 1 and 300 ng eGFP mRNA was added to each well with 100 μL full serum medium with 20,000 cells per well. GFP-positive cells were quantified by FACS (Attune) analysis 24 after treatment.

### Synthesis of Cationic and Ioniable Lipids Via Solid Phase Peptide Synthesis

Conventional chemical de novo synthesis cationic and ioniable is slow, expensive and inefficient. We demonstrate that cationic lipids and ionizable lipids can be synthesized using standard solid phase peptide synthesis methodology, e.g. Fields C G et al., Peptide Rescarch, 1 Mar. 1991, 4(2): 95-101, PMID: 1815783, HBTU activation for automated Fmoc solid-phase peptide synthesis; Behrendt et al, J. Pept. Sci. 2016; 22:4-27, Advances in Fmoc solid-phase peptide synthesis. We also disclose integration in an automated robotic system (ARS) of: (i) the solid phase lipid synthesis, (ii) initial cell screening, and (iii) animal organ or cell targeting.

This methodology has several advantages over conventional organic synthesis of cationic lipids and expands the types of cationic/ionizable lipids and lipid libraries that can be synthesized. It does not require a trained organic chemist and can be performed automatically and robotically and lowers the cost, time and chemical space that can be accessed, typically by a factor of at least 10 or 100.

### Rapidly Degrading Acid-Degradable Lipid Nanoparticles (RD-LPNs) Enhance the Delivery of mRNA in Vivo

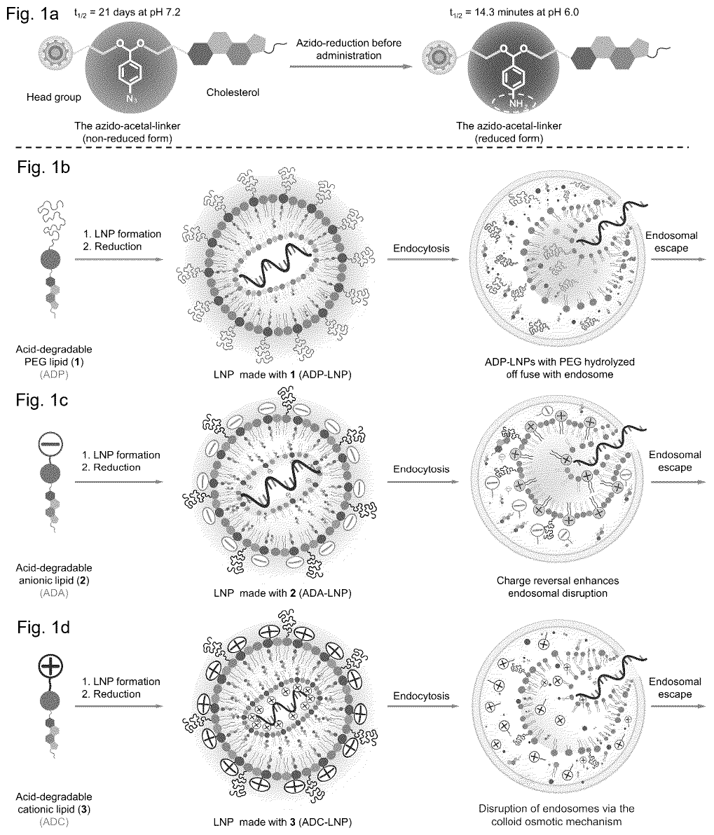

This example provides an acid-degradable linkers, termed azido-acetal that solves the problems associated with developing linkers that rapidly degrade in endosomes, and hydrolyzes in endosomes within minutes but are stable at pH 7.4 for 21 days. The azido-acetal linker is composed of a benzaldehyde acetal that has an azido in its para position and hydrolyzes via a two-step mechanism that requires reduction followed by acid hydrolysis (see FIG. 1). This two-step hydrolysis mechanism allows the azido-acetal to have a unique combination of stability and rapid hydrolysis at mildly acidic pHs. The azido-acetal has a slow hydrolysis rate due to the weak electron-withdrawing character of the azido (Hammett sigma value=0.1)¹⁷, enabling the synthesis of acid-degradable lipids in high yields and their incorporation into LNPs in aqueous environments. However, before administration, the azido-acetal is reduced to an amine via the addition of thiols. This reduction accelerates the azido-acetal's hydrolysis rate because of the strong electron-donating character of the amine (Hammett sigma value=−0.66)^{17, 18}.

We demonstrate here that the azido-acetal linker can be used as a platform to generate RD-LNPs and further demonstrate that RD-LNPs are superior to traditional LNPs with regards to delivering mRNA to the liver, lung, spleen, and brains of mice and to HSPCs in vitro. For example, RD-LNPs designed to deliver mRNA to the lungs rescued mice from acute lung injury via the delivery of IL-22 mRNA, whereas traditional LNPs could not. In addition, RD-LNPs that contained high levels of PEGylation efficiently delivered Cas9 mRNA and gRNA to brain tissue, and edited 5 times the number of brain cells as traditional LNPs. Collectively these studies demonstrate that engineering LNP hydrolysis rates in vivo expands the medical applications of LNPs.

### Results and Discussion

RD-LNPs are based upon the rapid hydrolysis of the azido-acetal linker at pH 6.0. We synthesized the model azido-acetal 4 and measured its acid-catalyzed hydrolysis rate before and after reduction to investigate whether reductive activation accelerates the hydrolysis kinetics of an azido-acetal. Table SI demonstrates that the reduction of an azido-acetal dramatically increases its hydrolysis rate. For example, compound 4 experienced under 1% hydrolysis after 6.5 hours at pH 7.4 and had a predicted hydrolysis half-life of 21 days at pH 7.4 at 37° C. However, after reduction, the reduced form of 4 hydrolyzed at pH 7.4 with a hydrolysis half-life of 2 hours, and at pH 6.0, its hydrolysis half-life was 14.8 minutes. The hydrolysis half-life of the azido-acetal was accelerated by a factor of 255 after reduction. The azido-acetal has the stability needed for performing multi-step organic reactions and should enable the development of new materials that are hypersensitive to acidic conditions. At pH 7.4, 4 is more stable than mRNA and has a hydrolysis half-life similar to the ester linkages found in LNP forming lipids and has the aqueous stability needed for formulating and storing LNPs. In addition, the reduced azido-acetal hydrolyzes with a t_{1/2}<30 minutes at pH 6.0 and has the hydrolysis kinetics needed to degrade in endosomes before trafficking to lysosomes occurs4. The two-step hydrolysis mechanism of the azido-acetal overcomes the stability problems preventing the development of rapidly hydrolyzing lipids and provides a general strategy for developing lipids and materials that rapidly hydrolyze at endosomal pHs.

We used the azido-acetal to synthesize the acid-degradable PEG-lipid 1, which had a hydrolysis half-life similar to 4 (sce Table S2). Engineering PEG-lipids to rapidly hydrolyze in endosomes has the potential to generate LNPs that have high levels of PEGylation and also transfect cells efficiently^{10, 19, 20}. The PEG-lipid reduces LNP toxicity and enhances LNP diffusion through tissue. However, LNPs are currently made with 0.5-1.5 mole percent of the PEG-lipid because increasing PEGylation levels beyond these levels can have detrimental effects. For example, the PEG-lipid lowers endosomal disruption by preventing the mixing of the ionizable lipid with endosomal lipids^{20, 21} and causes the formation of anti-PEG antibodies²². LNPs made with acid-degradable PEG-lipids should transfect cells efficiently even at high levels of PEGylation because they will hydrolyze off the LNP after endocytosis and will be released from the LNP surface. In addition, the immunogenicity of PEG dramatically increases after conjugation to lipids, and acid-degradable PEG-lipids may also have lower immunogenicity than traditional PEG-lipids because they will rapidly generate free PEG after endocytosis^{22, 23}.

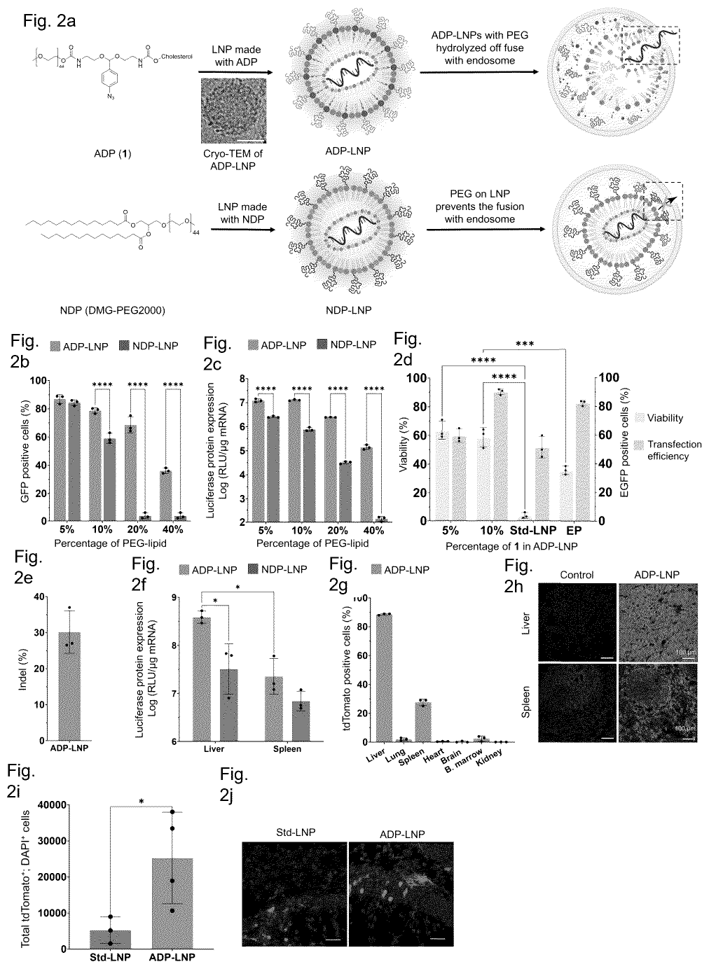

We investigated if LNPs made with high levels of 1 (termed ADP-LNPs) and EGFP mRNA could efficiently transfect HEK 293T cells. LNPs were formulated with mole percentages of 1 ranging from 5%-40% and their transfection ability was compared against LNPs made with a non-degradable PEG2k-cholesterol conjugate (termed NDP-LNPs) (sec FIG. 2a, and Chemical Structures I, II. The results are shown in FIG. 2b and demonstrated that ADP-LNPs tolerate high levels of PEGylation. For example, ADP-LNPs were efficient at transfecting HEK 293T cells at 10 and 20 mole percent and even had moderate efficiency at 40 mole percent. In contrast, NDP-LNPs were ineffective at 20 mole percent and could not transfect cells at high levels of PEGylation. The total amount of protein generated after treating cells with ADP-LNPs and NDP-LNPs containing luciferase mRNA was investigated to determine if rapid hydrolysis of the PEG-lipid increased the mRNA delivery efficiency. FIG. 2c demonstrates that cells treated with ADP-LNP/luciferase mRNA had significantly more luciferase activity than NDP-LNPs. The ability of ADP-LNPs to escape endosomes was also investigated via fluorescent microscopy using the Galectin-8 reporter cell line²⁴. The results demonstrated that ADP-LNPs can escape endosomes more efficiently than NDP-LNPs. The morphology and size of ADP-LNPs were investigated via Cryo-TEM and SAXS, this analysis demonstrated they had a size between 50-70 nm and also had high levels of bilayer character. Finally, we investigated the stability of ADP-LNPs to determine if they had the shelf-life needed for product development. The transfection ability of ADP-LNPs was determined after storing at −80° C. or 4° C. for 1-60 days, and ADP-LNPs retained their transfection efficiency after storing at −80° C. or 4° C. Collectively, these results demonstrate that the acid degradability of ADP-LNPs plays an essential role in enabling efficient mRNA delivery and that they have the stability needed for product development.

We investigated if ADP-LNPs could transfect cells that conventional LNPs cannot, in particular, hematopoictic stem and progenitor cells (HSPCs). HSPC transfection is currently performed via electroporation, which frequently causes high levels of cell death, or is accomplished via infection with lentivirus, which has significant safety risks^{25. 26}. Although conventional LNPs can transfect various cell lines efficiently with mRNA, they struggle to transfect HSPCs because of their toxicity^{27. 28}.

We investigated if ADP-LNPs could deliver EGFP mRNA to HSPCs without inducing significant cell death. ADP-LNPs containing EGFP mRNA were incubated with HSPCs at various LNP/mRNA doses and analyzed using flow cytometry. The results showed that ADP-LNPs achieved 90% transfection of HSPCs while maintaining 60% cell viability at a dose of 1 μg/mL of mRNA (FIG. 2d). In contrast, traditional LNPs caused high levels of toxicity to HSPCs and were only able to transfect 20% of the HSPCs due to dose-limiting toxicity. We anticipate that the lower toxicity of the ADP-LNPs is due to their high levels of PEGylation, which should shield their positive charges from the HSPC cell membrane29. In addition, we also compared the transfection efficacy of ADP-LNPs with electroporation. Electroporation of HSPCs with GFP mRNA transfected 82% of the cells but killed more than 65% of the cells (FIG. 2d).

ADP-LNPs were able to transfect HSPCs better than traditional LNPs and electroporation and we therefore investigated if they could deliver Cas9 mRNA and gRNA to HSPCs and edit HSPCs. Cas9 mRNA and sgRNA targeting AAVS1 were co-delivered to HSPCs with ADP-LNPs. FIG. 2e demonstrates that ADP-LNPs were able to deliver Cas9 mRNA and gRNA and generated 30% indels in HSPCs. ADP-LNPs have great potential as a transfection reagent for generating HSPC-based cell therapy products given their low toxicity and high transfection efficiency.

Given the complexity of the in vivo environment and the lack of correlation between in vitro and in vivo data, we next investigated whether ADP-LNPs could deliver mRNA in vivo to mice and compared their efficacy against LNPs that contained DMG-PEG (Std-LNPs) and NDP-LNPs. We first performed a pharmacokinetic study with fluorescently labeled ADP-LNPs to determine if the dense PEGylation of the ADP-LNPs increased their circulation half-life in comparison to Std-LNPs^{30. 31}. Results demonstrates that the circulation half-life of ADP-LNPs was significantly longer than Std-LNPs and was over 47 minutes, in contrast, Std-LNPs had a half-life of 6 minutes (sec Table S11). In a separate set of experiments, ADP-LNPs containing luciferase mRNA were injected intravenously via the retro-orbital route and the various organs were analyzed via ex-vivo imaging for luciferase transfection. Ex-vivo imaging demonstrated that ADP-LNPs transfected primarily the liver and spleen with luciferase mRNA and that ADP-LNPs delivered 9 and 4 times more luciferase mRNA to the liver and spleen than NDP-LNPs (FIG. 2f). Finally, we investigated the immunogenic toxicity of ADP-LNPs and compared them with Std-LNPs. Mice were injected with LNPs at a dose of 20 μg mRNA and after 24 hours, cytokines in the serum were analyzed, in particular IL-2, IL-6, MIP-2, and IFN-α. ADP-LNPs and Std-LNPs both caused very mild increases in IL-6 and MIP-2, and had no effect on the IL-2 and IFN-a levels, and were well tolerated after an intravenous injection.

Next, we performed experiments to determine the cell types transfected in vivo by the ADP-LNPs, using Ai9 mice with ADP-LNPs that contained Cre mRNA. ADP-LNPs were injected intravenously, and after 2 weeks, the organs were harvested and subjected to histology and flow cytometry analysis. The results revealed that ADP-LNPs transfected approximately 90% of the cells in the liver (FIG. 2g, 2h), and transfected major liver cell types, such as hepatocytes, macrophages, and endothelial cells. In addition, ADP-LNPs also transfected 25% of cells in the spleen (FIG. 2g, 2h), and specifically targeted macrophages, which are the target of numerous immuno-engineering-based therapeutics.

Finally, we investigated if the ADP-LNPs could efficiently transfect brain tissue after an intracranial injection. We chose the intracranial injection route as a platform to investigate the efficacy of ADP-LNPs because an intracranial injection overcomes the problems of the blood-brain barrier, and is currently the most effective method for delivering mRNA into the brain^{32. 33}. The extracellular matrix of the brain is ‘sticky’ and prevents nanoparticles from transfecting brain tissue efficiently after an intracranial injection, due to their limited diffusion in brain tissue. PEGylation can increase the diffusion of nanoparticles in brain tissues' and we therefore hypothesized that ADP-LNPs with 10% PEG would transfect brain tissue more efficiently than conventional LNPs with 1% PEG.

We employed the Ai9 mice model and Cre mRNA to assess the transfection efficiency and permeability of the ADP-LNPs. LNP/mRNA complexes were injected into the hippocampus and striatum and the transfection efficiency was evaluated by analyzing histology sections of the brain for tdTomato-positive cells. ADP-LNPs transfected the striatum and hippocampus after an intracranial injection with high efficiency and transfected approximately 30% of the cells in the striatum and hippocampus. In addition, ADP-LNPs diffused from the injection site in both the striatum and hippocampus and transfected millimeters of tissuc.

We performed additional experiments to investigate if ADP-LNPs could deliver Cas9 mRNA and gRNA into brain tissue, using the Ai9 mouse model and compared their delivery efficiency against standard LNPs (Std-LNPs), which contained about 1% PEG. Cas9 mRNA and gRNA were selected as mRNA cargo because of the great therapeutic potential of gene editing in the brain. ADP-LNPs were injected into the hippocampus and after 21 days analyzed for gene editing in the brain via counting of the red cells. FIG. 2i and FIG. 2j demonstrate that ADP-LNPs were significantly better at transfecting brain tissue with Cas9 mRNA and gRNA than Std-LNPs and transfected 5 times the number of cells in the brain as Std-LNPs. Collectively, the experiments with ADP-LNPs demonstrates they can deliver mRNA efficiently to HSPCs and brain tissue, and outperformed traditional methods due to their unique combination of dense PEGylation and rapid hydrolysis in endosomes.

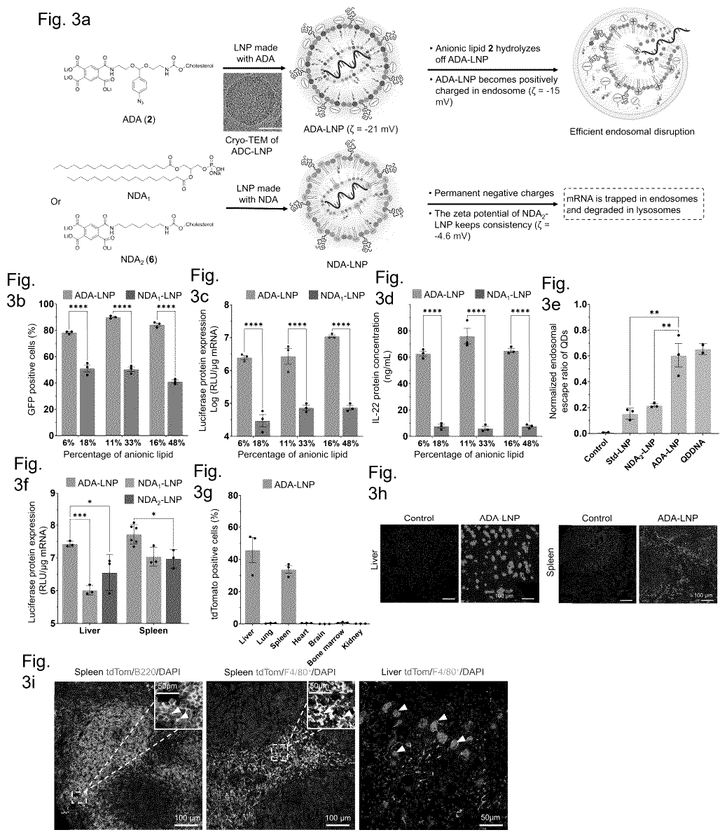

The versatility and chemical stability of the azido-acetal linker allowed us to develop other new classes of acid-degradable lipids. Anionic lipids were selected as the second lipid for engineering acid-catalyzed hydrolysis. LNPs that contain an anionic lipid have generated great interest because of their ability to target the spleen and mitigate the toxicity of LNPs by increasing their negative charge density^{34, 35}. However, LNPs with a negative charge density have low transfection efficiency because a positive surface charge density is required to trigger efficient cell uptake and endosomal disruption³⁶. To address this, we synthesized the anionic lipid 2, which contains 3 carboxylic acids connected to cholesterol via the azido-acetal linker. LNPs formulated with a high mole ratio of 2 (termed ADA-LNPs) are expected to possess a negative surface charge density and consequently target the spleen. ADA-LNPs are also engineered to trigger endosomal disruption. The hydrolysis of 2 in endosomes is anticipated to increase the osmolarity of the endosome by >33 mM, which would increase the osmolarity of the endosome by greater than 10% and induce osmotic destabilization of the endosome³⁷. In addition, the charge density of the ADA-LNPs will also become more positive in endosomes and this should facilitate their fusion with the anionic lipids in the endosomal membrane (FIG. 3a).

The transfection ability of ADA-LNPs was compared against LNPs made with the non-degradable anionic lipids C-18 phosphatidic acid (18PA, termed NDA₁-LNPs) and compound 6, which has a similar structure to 2, except that it lacks an acid degradable linkage (termed NDA₂-LNPs). The transfection efficiency of the ADA-LNPs was investigated in HEK 293T cells, using EGFP mRNA and luciferase mRNA. ADA-LNPs were significantly better at transfecting HEK 293T cells with EGFP mRNA than NDA₁-LNPs. For example, ADA-LNPs transfected >80% of cells whereas NDA₁-LNPs transfected only 50% of cells at maximum (FIG. 3b). In addition, ADA-LNPs were also able to deliver luciferase mRNA to HEK 293T cells more efficiently than NDA₁-LNPs and generated approximately 45-145 times higher levels of luciferase expression in cells than NDA₁-LNPs (FIG. 3c). ADA-LNPs also delivered IL-22 mRNA to cells more efficiently than NDA₁-LNPs and generated 8-13 times more IL-22 protein in the supernatant in ADA-LNP treated cells than NDA₁-LNPs (FIG. 3d). In addition, ADA-LNPs had a potential of −21 mV (Table S6), suggesting that they should have excellent biocompatibility.

We performed fluorescent microscopy experiments with ADA-LNPs containing quantum dot-DNA conjugates to determine if the rapid hydrolysis of 2 enhanced endosomal disruption. Quantum dots can image endosomal disruption by measuring intracellular diffusion coefficients and trajectories³⁸. Cells were exposed to quantum dots encapsulated within ADA-LNPs, NDA₁-LNPs, or Std-LNPs. Subsequently, continuous fluorescent microscopy was used to image the cells. FIG. 3e demonstrates that ADA-LNPs rapidly induced endosomal release of the quantum dot-DNA conjugates causing approximately 60% of the quantum dots to get released from the endosome. In contrast, NDA₁-LNPs and Std-LNPs largely remained in endosomes and only 20% of their encapsulated quantum dots were released from the endosome. The ability of ADA-LNPs to escape endosomes was further investigated via fluorescent microscopy using the Galectin-8 reporter cell line24. FIGS. 10-8 verifies the quantum dot results with ADA-LNPs and demonstrates that ADA-LNPs were able to permeabilize endosomes and disrupt them efficiently. These results demonstrate that RD-LNPs can efficiently trigger endosomal disruption and also enhance the mRNA delivery efficiency of anionic LNPs.

We performed experiments to investigate the ability of ADA-LNPs to deliver mRNA in vivo using luciferase mRNA and Cre mRNA. Mice were injected with ADA-LNPs encapsulating luciferase mRNA and analyzed through whole-body and ex-vivo imaging. FIG. 3f demonstrates that ADA-LNPs generated luciferase activity in the spleen that was approximately 8 times higher than NDAI-LNPs or NDA₂-LNPs. The cell tropism and transfection efficiency of ADA-LNPs were assessed by delivering Cre mRNA to Ai9 mice. ADA-LNPs were intravenously delivered to mice via the retro-orbital route and the liver and spleen were analyzed via histology and flow cytometry. ADA-LNPs transfected 32% of the cells in the spleen and 45% of the cells in the liver (FIG. 3g). Histological analysis (FIGS. 3h and 3i) indicated that ADA-LNPs primarily transfected macrophages in the spleen, amounting to approximately 25% of the macrophages. In addition, ADA-LNPs also transfected approximately 5% of the B cells in the spleen, while hepatocytes were the predominant cell type transfected in the liver. These experiments demonstrate the potential of ADA-LNPs as therapeutics for treating various immune disorders and for generating protein replacement therapies targeting the liver. Moreover, they demonstrate that the azido-acetal linker can serve as a platform for developing new acid-degradable lipids for mRNA delivery.

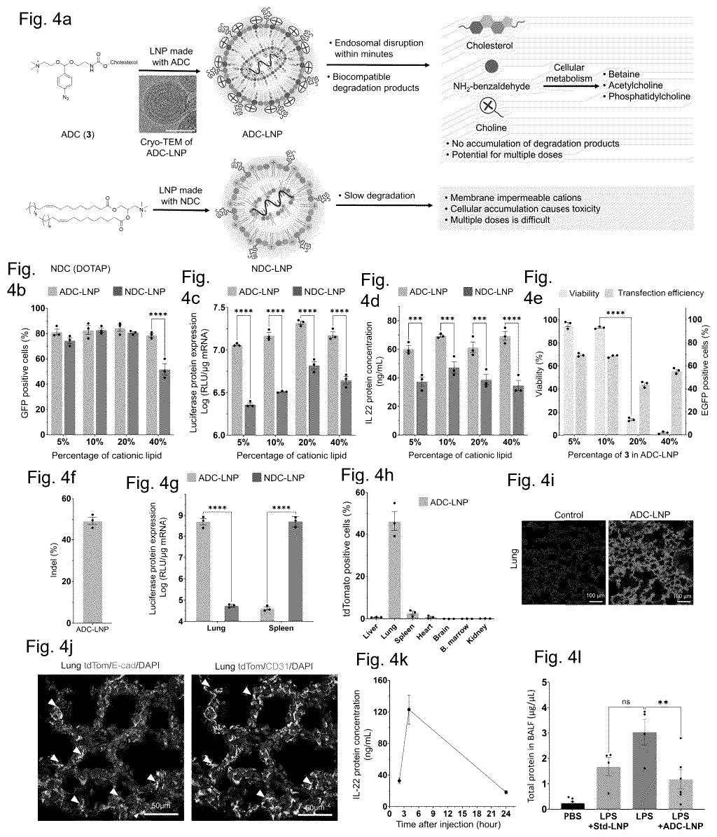

Finally, we investigated if engineering rapid hydrolysis in the endosome could improve the transfection efficiency of cationic lipids (FIG. 4a). LNPs containing cationic lipids have tremendous potential for transfecting lung tissue and are currently being explored as platforms for developing new lung therapeutics^{35, 39-42}. However, current cationic lipids have several problems that limit their clinical applications. Cationic lipids such as DOTAP and DDAB degrade slowly in cells and generate membrane impermeable cations, which will likely stay trapped within cells¹³. Consequently, treating patients with multiple doses of cationic lipids is challenging because of their tissue accumulation. In addition, cationic lipids generate toxicity because of their positive charges, and having them persist within cells for long periods of time further increases their toxicity⁴³. There are consequently no FDA-approved LNP formulations that have permanent positive charges.

The acid-degradable cationic lipid 3 has the potential to address several of the problems limiting the development of cationic lipids. 3 hydrolyzes in cells orders of magnitude faster than conventional cationic lipids and degrades into biocompatible or membrane permeable products, which will be rapidly eliminated from cells and tissues. 3 degrades into choline, 4-amino-benzaldehyde and cholesterol on the timescale of minutes at pH 6.0. Choline is an essential metabolite that is incorporated into a variety of metabolic pathways, such as the biosynthesis of phosphatidylcholine, trimethyl glycine (betaine), and acetylcholine, and is an essential food source for cells. In addition, cholesterol is a natural lipid^{44, 45}, and 4-amino benzaldehyde is found in a variety of foods, such as yellow bell pepper, and can be ingested with minimal toxicity. LNPs made with 3 (termed ADC-LNPs) are designed to have mitigated toxicity because of their rapid intracellular degradation and biocompatible degradation products and should be amenable to repeat dosing for chronic conditions. In addition, 3 is also expected to enhance the endosomal release of mRNA because its hydrolysis in endosomes will increase the osmolarity of the endosome, resulting in endosomal disruption via the colloid osmotic effect. Finally, ADC-LNPs also rapidly decomplex mRNA in the endosomes, due to the decomposition of the cationic lipid, and this further increase their transfection efficiency.

ADC-LNPs were formulated with EGFP mRNA that contained various mole ratios of 3 and their ability to transfect HEK 293T cells was investigated. FIG. 4b demonstrates that ADC-LNPs tolerate large mole ratios of 3 and efficiently transfected cells even at 40% mole ratios. The azido-acetal linker can therefore generate diverse classes of lipids ranging from PEG-lipids to cationic lipids and should be able to engineer acid sensitivity in a wide range of new lipid-based compounds. Subsequently, the mRNA transfection efficiency of ADC-LNPs was investigated in HEK 293T cells, using LNPs that encapsulated luciferase mRNA and IL-22 mRNA, and compared against LNPs made with DOTAP (termed NDC-LNPs). FIG. 4c and FIG. 4d demonstrate that LNPs with 40 mole percent of 3 can deliver mRNA to HEK 293T cells better than NDC-LNPs. ADC-LNPs produced approximately 4 times the luciferase signal and 2 times more IL-22 protein than NDC-LNPs. We also investigated the ability of ADC-LNPs to escape endosomes via fluorescent microscopy using the Galectin-8 reporter cell line. Results demonstrate that ADC-LNPs can permeabilize endosomes and induce endosomal escape.

We investigated if ADC-LNPs could transfect human HSPCs. HSPCs were treated with ADC-LNPs complexed with EGFP mRNA at a 3 μg/mL mRNA concentration and the transfection efficiency and toxicity were determined by flow cytometry. FIG. 4e demonstrates that ADC-LNPs can transfect HSPCs with low toxicity. ADC-LNPs containing 5 or 10 mole percent of 3 transfected 70% of HSPCs and had nearly no cytotoxicity. ADC-LNPs performed significantly better than Std-LNPs, which only generated 20% transfection but caused >95% toxicity. In addition, we also investigated if ADC-LNPs could deliver Cas9 mRNA and gRNA to HSPCs. ADC-LNPs made with 10% of 3 were used to deliver Cas9 mRNA and gRNA targeting the AAVS1 gene to HSPCs and generated 48% indels, demonstrating they have potential as a platform for developing new cell-based therapies (FIG. 4f). In addition, 3 has the potential to be an alternative to DOTAP, which efficiently delivers mRNA to cells and rapidly hydrolyzes into biocompatible degradation products.

Mouse experiments were performed with ADC-LNPs containing 40 mole percent of 3and luciferase mRNA to determine if LNPs made with high mole ratios of 3 could transfect lung tissue with selectivity. ADC-LNPs were injected into mice and luciferase activity in the lung and liver was analyzed. ADC-LNPs generated a lung signal close to 109 photons/sec/cm²/sr (FIG. 4g). In contrast NDC-LNPs containing 40 mole percent of DOTAP primarily transfected the spleen. A Cre mRNA delivery experiment using Ai9 mice was performed to identify the cell types and the percentage of cells transfected in the lungs. Ai9 mice were injected with ADC-LNPs and the lung tissue was analyzed 2 weeks later via histology to determine the anatomical location of the transfected cells and their cell type. In addition, flow cytometry was also performed to determine the percentage of cells transfected in the lungs. FIG. 4h shows that ADC-34 LNPs transfected approximately 50% of the cells in the lung after a single injection, which is sufficient to enable the development of a variety of therapeutics. Histological analysis of the lung tissue demonstrated that the transfected cells were predominantly in the smaller airways localized around the alveolus, and in this region the transfection rates were >70% (FIG. 4i, 4j). In addition, epithelial cells and endothelial cells in the alveolus were efficiently transfected. A variety of devastating diseases such as COPD, acute lung injury and lung cancer originate from pathologies in endothelial and epithelial cells located in the alveolus⁴⁶. ADC-LNPs, therefore, provide a platform for developing new lung therapeutics.

We used ADC-LNPs to develop a potential therapeutic for acute lung injury based on delivering IL-22 mRNA to the lung. IL-22 can treat a variety of lung inflammatory diseases, via activation of the STAT 3 signaling pathway in lung epithelial cells47-49, but has been challenging to develop because of its high toxicity and short circulation half-life (under 2 hours) 50. Lung targeted LNPs have the potential to localize IL-22 in the lung and thereby generate a higher therapeutic window than soluble IL-22 protein, which has no targeting ability. IL-22 mRNA was encapsulated into ADC-LNPs and the ability of ADC-LNPs to deliver IL-22 mRNA in vivo and generate therapeutic concentrations of IL-22 protein in the blood via lung transfection was investigated via ELISA at 2, 4, and 24 hours after administration. FIG. 4k shows that ADC-LNPs generated a peak IL-22 concentration of 120 ng/mL after 4 hours and maintained a therapeutic concentration in the blood for >24 hours with a single injection of LNPs. Based on this, we investigated if ADC-LNPs containing IL-22 mRNA could rescue mice from acute lung injury and compared their therapeutic efficacy against Std-LNPs that primarily transfect the liver. Mice were given intravenous injections of ADC-LNPs or control LNPs and then challenged with an intratracheal injection of LPS. 4 days after LPS treatment, the mice were sacrificed and the protein content in the lung was analyzed. FIG. 4l demonstrates that ADC-LNPs significantly reduced the protein content in the lung in comparison to LPS-treated mice, whereas Std-LNPs were unable to do so. ADC-LNPs containing cytokine mRNA have significant benefits over Std-LNPs and should enable the development of new lung therapeutics. Finally, these experiments further validate the azido-acetal linker as a platform for developing RD-LNPs and new acid-degradable materials.

The invention provides a new class of LNPs that rapidly hydrolyze in endosomes, termed RD-LNPs. The synthesis of the RD-LNPs was facilitated by the development of the azido-acetal linker, which has a hydrolysis half-life of 21 days at pH 7.4 but only 14.8 minutes at pH 6.0 (after reduction). RD-LNPs containing 1, 2, or 3 enhanced the delivery of mRNA across a variety of cell types and organs, and outperformed traditional LNPs in multiple organs. For example, RD-LNPs transfected the brain and lung in vivo with therapeutic mRNAs significantly better than traditional LNPs and also transfected HSPCs better than traditional LNPs or electroporation. RD-LNPs require mixing with a thiol reducing agent such as DTT, before administration. A variety of thiol-reducing agents, such as □-lipoic acid, are present in commonly used dietary supplements and are routinely ingested by people. □-Lipoic acid has a reducing power similar to DTT and can be used to activate the RD-LNPs.

### REFERENCES

1. Tenchov, R., et al. PEGylated lipid nanoparticle formulations: immunological safety and efficiency perspective. Bioconjug. Chem. 34, 941-960 (2023).
2. Müller, S. S. et al. Biodegradable hyperbranched polyether-lipids with in-chain pH-sensitive linkages. Polym. Chem. 7, 6257-6268 (2016).
3. Kedmi, R., et al. The systemic toxicity of positively charged lipid nanoparticles and the role of Toll-like receptor 4 in immune activation. Biomater. 31, 6867-6875 (2010).
4. Huotari, J. et al. Endosome maturation.EMBO J. 30, 3481-3500 (2011).
5. Chen, H., et al. Novel pH-sensitive cationic lipids with linear ortho ester linkers for gene delivery. Eur. J. Med. Chem. 52, 159-172 (2012).
6. Palanikumar, L. et al. pH-responsive high stability polymeric nanoparticles for targeted delivery of anticancer therapeutics. Commun. Biol. 3, 95 (2020).
7. Gao, W., Chan, J. M. & Farokhzad, O. C. pH-Responsive nanoparticles for drug delivery. Mol. Pharm. 7, 1913-1920 (2010).
8. Deirram, N., Zhang, C., Kermaniyan, S. S., Johnston, A. P. R. & Such, G. K. pH-Responsive Polymer Nanoparticles for Drug Delivery. Macromol. Rapid Commun. 40, c1800917 (2019).
9. Zhuo, S. et al. pH-Sensitive biomaterials for drug delivery. Molecules 25 (2020).
10. Shin, J., et al. Acid-triggered release via dePEGylation of DOPE liposomes containing acid-labile vinyl ether PEG-lipids. J. Control. Release 91, 187-200 (2003).
11. Guo, X. & Szoka, F. C. Steric stabilization of fusogenic liposomes by a low-pH sensitive PEG-diortho ester-lipid conjugate. Bioconjug. Chem. 12, 291-300 (2000).
12. Jayaraman, M. et al. Maximizing the potency of siRNA lipid nanoparticles for hepatic gene silencing in vivo. Angew. Chem. Int. Ed. Engl. 51, 8529-8533 (2012).
13. Zhang, Y., Sun, C., Wang, C., Jankovic, K. E. & Dong, Y. Lipids and lipid derivatives for RNA delivery. Chem. Rev. 121, 12181-12277 (2021).
14. Roise, J. J. et al. Acid-sensitive surfactants enhance the delivery of nucleic acids. Mol. Pharm. 19, 67-79 (2022).
15. Yang, X. et al. Making smart drugs smarter: The importance of linker chemistry in targeted drug delivery. Med. Res. Rev. 40, 2682-2713 (2020).
16. Liu, B. & Thayumanavan, S. Substituent effects on the pH sensitivity of acetals and letals and their correlation with encapsulation stability in polymeric nanogels. J. Am. Chem. Soc. 139, 2306-2317 (2017).
17. Hansch, C., Leo, A, Taft, R. W. A survey of Hammett substituent constants and resonance and field parameters. Chem. Rev. 91, 165-195 (1991).
18. Takahata, Y. & Chong, D. P. Estimation of Hammett sigma constants of substituted benzenes through accurate density-functional calculation of core-electron binding energy shifts. Int. J. Quantum Chem. 103, 509-515 (2005).
19. Coelho, F., Salonen, L. M. & Silva, B. F. B. Hemiacetal-linked pH-sensitive PEG-lipids for non-viral gene delivery. New J. Chem. 46, 15414-15422 (2022).
20. Fang, Y. et al. Cleavable PEGylation: a strategy for overcoming the “PEG dilemma” in efficient drug delivery. Drug Deliv. 24, 22-32 (2017).
21. Chan, C. L. et al. Endosomal escape and transfection efficiency of PEGylated cationic liposome-DNA complexes prepared with an acid-labile PEG-lipid. Biomater. 33, 4928-4935 (2012).
22. Kozma, G. T., Shimizu, T., Ishida, T. & Szebeni, J. Anti-PEG antibodies: Properties, formation, testing and role in adverse immune reactions to PEGylated nano-biopharmaceuticals. Adv. Drug. Deliv. Rev. 154-155, 163-175 (2020).
23. Wang, H. et al. Polyethylene glycol (PEG)-associated immune responses triggered by clinically relevant lipid nanoparticles in rats. NPJ Vaccines 8, 169 (2023).
24. Kilchrist, K. V. et al. Gal8 visualization of endosome disruption predicts carrier-mediated biologic drug intracellular bioavailability. ACS Nano 13, 1136-1152 (2019).
25. Schmiderer, L. et al. Efficient and nontoxic biomolecule delivery to primary human hematopoietic stem cells using nanostraws. Proc. Natl. Acad. Sci. U. S. A. 117, 21267-21273 (2020).
26. Levetzow, G. V. et al. Nucleofection, an efficient nonviral method to transfer genes into human hematopoietic stem and progenitor cells. Stem. Cells Dev. 15 (2006).
27. Vhora, I., Lalani, R., Bhatt, P., Patil, S. & Misra, A. Lipid-nucleic acid nanoparticles of novel ionizable lipids for systemic BMP-9 gene delivery to bone-marrow mesenchymal stem cells for osteoinduction. Int. J. Pharm. 563, 324-336 (2019).
28. Shi, D., Toyonaga, S. & Anderson, D. G. In vivo RNA delivery to hematopoietic stem and progenitor cells via targeted lipid nanoparticles. Nano Lett. 23, 2938-2944 (2023).
29. Kumar, V. et al. Shielding of lipid nanoparticles for siRNA delivery: impact on physicochemical properties, cytokine induction, and efficacy.Mol. Ther. Nucleic Acids 3, e210 (2014).
30. Sakurai, Y. et al. Efficient siRNA delivery by lipid nanoparticles modified with a nonstandard macrocyclic peptide for EpCAM-targeting. Mol. Pharm. 14, 3290-3298 (2017).
31. Chander, N., et al. Lipid nanoparticle mRNA systems containing high levels of sphingomyelin engender higher protein expression in hepatic and extra-hepatic tissues.Mol. Ther. Methods Clin. Dev. 30, 235-245 (2023).
32. Ruoslahti, E. Brain extracellular matrix. Glycobiol. 6, 489-492 (1996).
33. Dankovich, T. M. et al. Extracellular matrix remodeling through endocytosis and resurfacing of Tenascin-R. Nat. Commun. 12, 7129 (2021).
34. Cheng, Q. et al. Selective organ targeting (SORT) nanoparticles for tissue-specific mRNA delivery and CRISPR-Cas gene editing. Nat. Nanotechnol. 15, 313-320 (2020).
35. LoPresti, S. T., Arral, M. L., Chaudhary, N. & Whitehead, K. A. The replacement of helper lipids with charged alternatives in lipid nanoparticles facilitates targeted mRNA delivery to the spleen and lungs. J. Control. Release 345, 819-831 (2022).
36. Frohlich, E. The role of surface charge in cellular uptake and cytotoxicity of medical nanoparticles. Int. J. Nanomedicine 7, 5577-5591 (2012).
37. Hu, M., Zhou, N., Cai, W. & Xu, H. Lysosomal solute and water transport. J. Cell Biol. 221 (2022).
38. Zahid, M. U., Ma, L., Lim, S. J. & Smith, A. M. Single quantum dot tracking reveals the impact of nanoparticle surface on intracellular state. Nat. Commun. 9, 1830 (2018).
39. Pei, Y. et al. Synthesis and bioactivity of readily hydrolysable novel cationic lipids for potential lung delivery application of mRNAs. Chem. Phys. Lipids 243, 105178 (2022).
40. Qiu, M. et al. Lung-selective mRNA delivery of synthetic lipid nanoparticles for the treatment of pulmonary lymphangioleiomyomatosis. Proc. Natl. Acad. Sci. U. S. A. 119 (2022).
41. Lokugamage, M. P. et al. Optimization of lipid nanoparticles for the delivery of nebulized therapeutic mRNA to the lungs. Nat. Biomed. Eng. 5, 1059-1068 (2021).
42. Li, Q. et al. Engineering caveolae-targeted lipid nanoparticles to deliver mRNA to the lungs. ACS Chem. Biol. 15, 830-836 (2020).
43. Landesman-Milo, D. & Peer, D. Toxicity profiling of several common RNAi-based nanomedicines: a comparative study. Drug. Deliv. Transl. Res. 4, 96-103 (2014).
44. Sanders, L. M. & Zeisel, S. H. Choline: Dietary requirements and role in brain development. Nutr. Today 42, 181-186 (2007).
45. Gibellini, F. & Smith, T. K. The Kennedy pathway--De novo synthesis of phosphatidylethanolamine and phosphatidylcholine. IUBMB Life 62, 414-428 (2010).
46. Raghu, G., Nyberg, F. & Morgan, G. The epidemiology of interstitial lung disease and its association with lung cancer. Br. J. Cancer 91 Suppl 2, S3-10 (2004).
47. McAleer, J. P. & Kolls, J. K. Directing traffic IL-17 and IL-22 coordinate pulmonary immune defense. Immunol. Rev. 260, 129-144 (2014).
48. Muhl, H. et al. IL-22 in tissue-protective therapy. Br. J. Pharmacol. 169, 761-771 (2013).
49. Mizoguchi, A. et al. Clinical importance of IL-22 cascade in IBD. J. Gastroenterol. 53, 465-474 (2018).
50. Hwang, S., Feng, D. & Gao, B. Interleukin-22 acts as a mitochondrial protector. Theranostics 10, 7836-7840 (2020).

### Supplemental Information

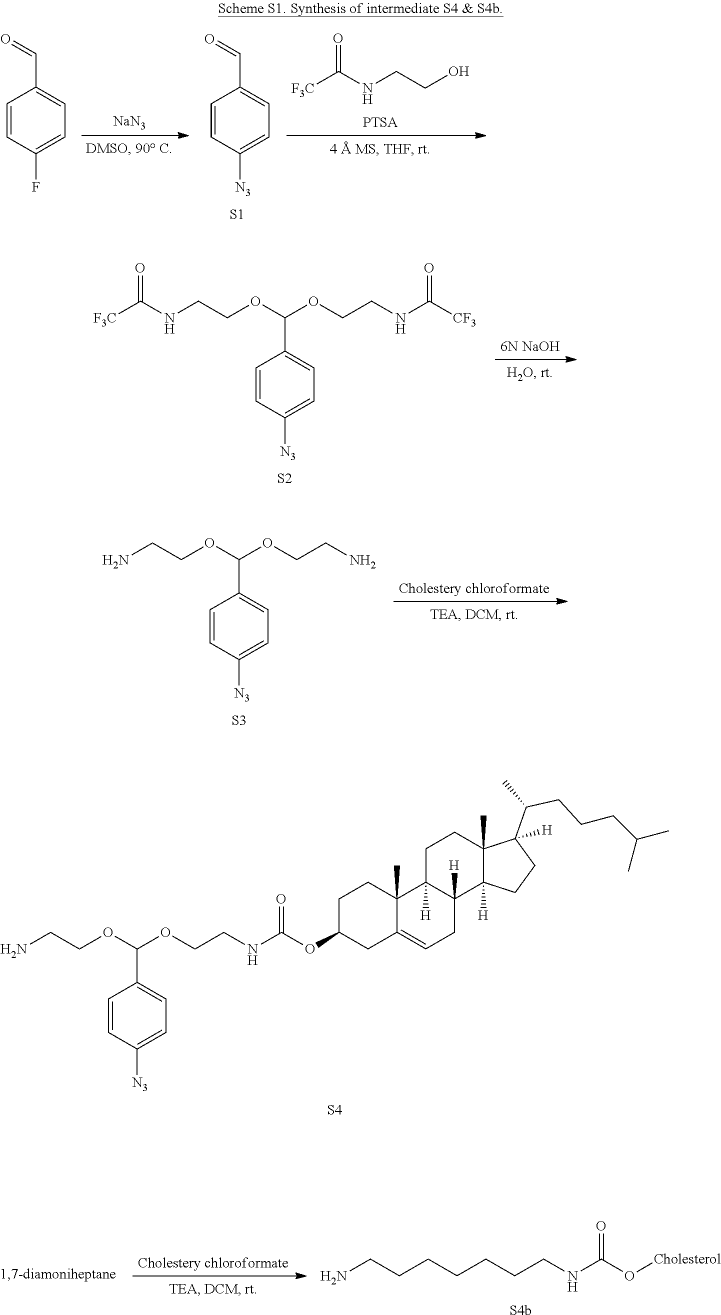

### Synthesis of Compound S2¹

To a solution of S12 (4.0 g, 27.19 mmol), trifluoroacetamide protected amino alcohol (18.6 g, 108.7 mmol, 4.0 equiv), 5 Å molecular sieves (30 g, activated before use by placing in an oven for 5 days and flame drying), were dissolved in dry tetrahydrofuran (40 mL) under a nitrogen atmosphere. After stirring under nitrogen for 30 minutes, concentrated p-Toluene sulfonic acid (380 μL, 4 mmol, 0.15 equiv) was added. In general, the reaction completes within 2 hours at room temperature. The reaction was quenched with triethylamine (4 mL) and the molecular sieves were removed by filtration. The solvent was removed under reduced pressure and S2 was purified by recrystallization with hexane and ethyl acetate to give S2 as an off-white powder (9 g, 75%). TLC (Hexane: Ethylacetate, 2:1 v/v): Rf=0.3. ¹H NMR (400 MHZ, CDCl₃) δ 7.37 (d, J=8.5 Hz, 2H), 7.04 (d, J=8.5 Hz, 1H), 5.50 (s, 1H), 3.72-3.47 (m, 8H). ¹³C NMR (101 MHZ, CDCl₃) δ 157.4 (q, J_{C-F}=148), 141.1, 133.6, 127.9, 119.2, 101.8, 101.4 (q, J_{C-F}=4412), 63.5, 39.7. HRMS (m/z): [M+Na]⁺ calcd. for C₁₅H₁₅F₆N₅Na, 466.0926; found, 466.0921.

### Synthesis of Compound S3¹

To 100 mL of 6 M NaOH was added S2 (8 g, 18.06 mmol, 1.0 equiv). The reaction mixture was stirred at room temperature for 4 hours and the starting materials had completely disappeared (by TLC). The reaction mixture was extracted with dichloromethane (50 mL×5). The organic phase was dried over MgSO₄ and concentrated under reduced pressure. The resulting crude S3 was used in the next step without further purification. TLC (Ethylacetate: MeOH: Ammonium hydroxide, 3:1:1 v/v/v): R_{f}=0.5. ¹H NMR (400 MHZ, CDCl₃) δ 7.44 (d, J=8.6 Hz, 2H), 7.01 (d, J=8.5 Hz, 2H), 5.53 (s, 1H), 3.58 (dt, J=10.3, 5.2Hz, 2H), 3.49 (dt, J=10.0, 5.3 Hz, 2H), 2.89 (t, J=5.3 Hz, 4H). ¹³C NMR (101 MHZ, CDCl₃) δ 140.2, 135.3, 128.2, 118.9, 101.4, 67.7, 41.9. HRMS (m/2): [M+Na]⁺ calcd. for C₁₁H₁₇N₅NaO₂, 274.1280; found, 274.1274.

### Synthesis of Compound S4

To a solution of S3 (2.0 g, 7.97 mmol, 1.0 equiv) in 50 mL dichloromethane, was added a solution of cholesteryl chloroformate (2.5 g, 5.56 mmol, 0.7 equiv, dissolved in 40 mL DCM) dropwise in an ice bath. After completing the addition of cholesteryl chloroformate, the reaction mixture was allowed to warm to room temperature and stirred in an ice bath for 2 additional hours. The reaction mixture was concentrated under reduced pressure and the residue was purified on a Biotage Selekt using MeOH/DCM (0.1% TEA): (0-10%) to give S4 (2.6 g, 68%). TLC (DCM: McOH, 10:1 v/v): R_{f}=0.4. ¹H NMR (400 MHZ, CDCl₃) δ 7.43 (d, J=8.5 Hz, 2H), 7.02 (d, J=8.5 Hz, 2H), 5.52 (s, 1H), 5.36 (d, J=5.0 Hz, 1H), 5.17 (t, J=5.9 Hz, 1H), 4.48 (tt, J=10.9, 4.7 Hz, 1H), 3.67-3.45 (m, 4H), 3.43-3.26 (bm, 2H), 3.04-2.77 (bm, 2H), 2.38-2.17(m, 2H), 2.05-0.92 (m, 33H), 0.91 (d, J=6.5 Hz, 3H), 0.86 (dd, J=6.7, 1.8 Hz, 6H), 0.67 (s, 3H). ¹³C NMR (101 MHZ, CDCl₃) δ 156.2, 140.4, 139.8, 135.0, 128.2, 122.5, 118.9, 101.4, 74.4, 67.9, 64.6, 56.7, 56.2, 50.0, 42.3, 41.8, 40.9, 39.8, 39.5, 38.6, 37.0, 36.6, 36.2, 35.8, 31.9, 31.9, 28.3, 28.2, 28.0, 24.3, 23.9, 22.8, 22.6, 21.1, 19.4, 18.7, 11.9. HRMS (m/z): [M+H]⁺ calcd. for C₃₉H₆₂N₅O₄, 664.4802; found, 664.4803.

### Synthesis of Compound S4b

The protocol is the same as S4b. TLC (DCM: McOH, 10:1 v/v): R=0.4. ¹H NMR (500 MHz, CDCl₃) δ 5.34-5.24 (m, 1H), 4.77 (t, J=6.1 Hz, 1H), 4.40 (tt, J=11.4, 4.7 Hz, 1H), 3.15-3.00 (m, 2H), 2.93 (brs, 2H), 2.35-2.12 (m, 2H), 2.00-0.86 (m, 39H), 0.84 (d, J=6.5Hz, 3H), 0.79 (dd, J=6.6, 2.3 Hz, 6H), 0.61 (s, 3H). ¹³C NMR (126 MHZ, CDCl₃) δ 156.26, 139.86, 122.46, 77.30, 77.25, 77.05, 76.79, 74.18, 56.70, 56.19, 50.00, 42.32, 40.83, 39.76, 39.53, 38.63, 37.02, 36.57, 36.21, 35.83, 31.90, 31.88, 29.87, 28.64, 28.25, 28.22, 28.01, 26.53, 26.42, 24.30, 23.89, 22.83, 22.57, 21.06, 19.37, 18.73, 11.87. HRMS (m/z): [M+H]⁺ calcd. for C₃₅H₆₃N₂O₂, 543.4884; found, 543.4880.

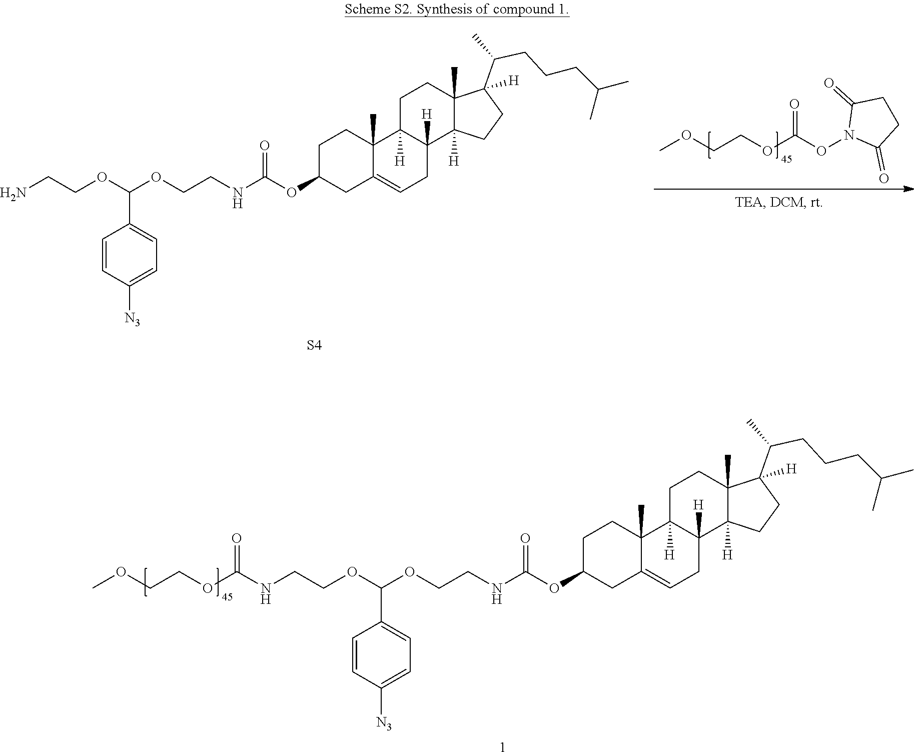

To a solution of S4 (70 mg, 0.105mmol, 1.0 equiv) in DMF (5.0 mL) was added mPEG2k succinimidyl carbonate (232 mg, 0.116 mmol, 1.1 equiv) followed by the addition of triethylamine (TEA) 30 μL, 0.211 mmol, 2 equiv). The reaction mixture was stirred at room temperature overnight. The solvent was removed under reduced pressure. The resulting residue was purified by preparative TLC to afford compound 1 (120 mg, 60%) as a white solid. TLC (DCM: MeOH, 10:1 v/v): Rf=0.5. ¹H NMR (500 MHz, CDCl₃) § 7.34 (d, J=8.5 Hz, 2H), 6.96 (d, J=8.5 Hz, 2H), 5.43 (s, 1H), 5.30 (dd, J=5.2, 2.1 Hz, 1H), 5.19 (bs, 1H), 5.00 (bs, 1H), 4.42 (tt, J=11.1, 4.6 Hz, 1H), 4.14 (t, J=4.7 Hz, 2H), 3.80-3.40 (m, 187H), 3.41-3.20 (m, 8H), 2.31-2.09 (m, 5H), 1.98-1.86 (m, 2H), 1.83-1.72 (m, 3H), 1.59-0.87 (m, 27H), 0.84 (d, J=6.5 Hz, 3H), 0.79 (dd, J=6.7, 2.3 Hz, 7H), 0.61 (s, 3H). ¹³C NMR (101 MHZ, CDCl₃) δ156.4, 156.1, 140.5, 139.8, 134.6, 128.2, 122.5, 118.9, 101.4, 74.4, 71.9, 70.6, 69.6, 64.7, 64.0, 59.0, 56.7, 56.1, 50.0, 42.3, 40.9, 40.8, 39.7, 39.5, 38.6, 37.0, 36.6, 36.2, 35.8, 31.9, 28.2, 28.2, 28.0, 24.3, 23.8, 22.8, 22.6, 21.1, 19.3, 18.7, 11.9.

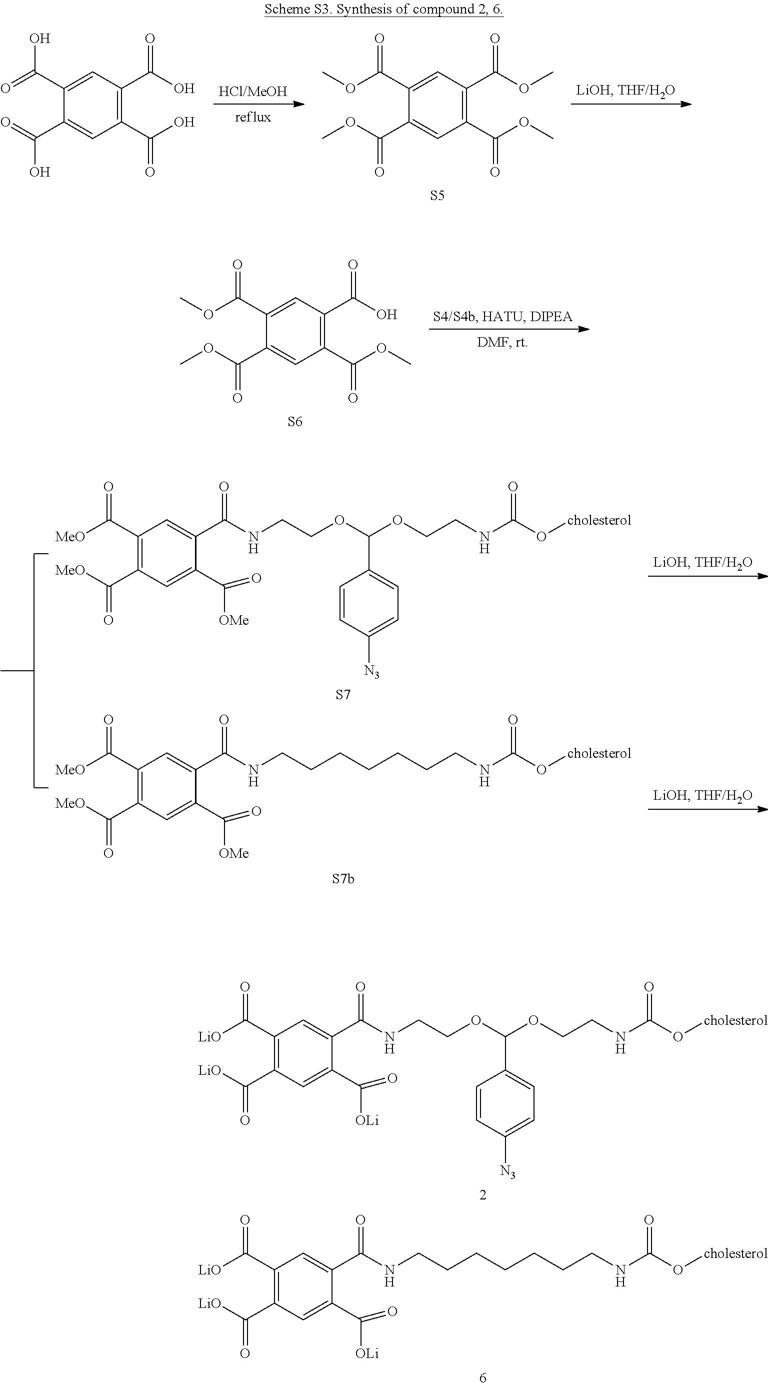

### Synthesis of Compound S5

Pyromellitic acid (5.0 g, 19.69 mmol, 1.0 equiv) was loaded in a round-bottom flask and suspended in McOH (50 mL) at 0° C. SOCl₂ (5.7 g, 78.7 mmol, 4 equiv) was added dropwisc via a syringe. The reaction mixture was heated under reflux overnight. After cooling to room temperature, the volatiles were removed. The crude mixture was dissolved in dichloromethane and washed with water (2×100 mL). The organic phase was then dried over MgSO₄, filtered, and evaporated to achieve S5 (quantitative). The crude product was washed with diethyl ether and used in the next step without further purification.

### Synthesis of Compound S6

To a solution of S5 (1.0 g, 3.22 mmol, 1.0 equiv) in 30 mL of THF/H2O (2:1) was added LiOH (77 mg, 3.22 mmol, 1.0 equiv). The reaction mixture was stirred at room temperature for 3 hours. The reaction was quenched with 0.5 M HCl (20 mL) and extracted with ethyl acetate (30 mL×3). The combined organic phase was washed with NaCl brine (30 mL×3), dried over MgSO₄, and the solvent was removed under reduced pressure. The residue was purified on a Biotage Selekt machine, using a silica gel column with an eluent of hexane and ethyl acetate, to generate S6 as a white solid (300 mg, 30%). TLC (Hexane: ethylacetate, 1:1 v/v): R_{f}=0.5.1H NMR (500 MHZ, CDCl₃) δ 10.10 (bs, 1H), 8.17 (s, 1H), 7.96 (s, 1H), 3.93-3.81 (m, 9H). 13C NMR (126 MHz, CDCl₃) δ 170.3, 166.7, 166.4, 166.2, 135.3, 135.2, 135.2, 133.9, 132.5, 130.4, 129.5, 53.3, 53.2, 53.2. HRMS (m/z): [M−H]⁻ calcd. for C₁₃H₁₁O₈, 295.0459; found, 295.0460.

### Synthesis of Compound S7

To a solution of S6 (50 mg, 0.076 mmol, 1.0 cquiv) and S4 (25 mg, 0.082 mmol, 1.1 equiv) in DMF (5 mL) at room temperature was added HATU (22 μL, 0.112 mmol, 1.5 equiv) and DIPEA (13 μL, 0.06 mol, 2.0 equiv). The reaction mixture was stirred at room temperature for 1 hour. After TLC showed the reaction was completed, the solvent was removed under reduced pressure. The resulting residue was purified by preparative TLC to afford S7 (45 mg, 63%) as a white powder. TLC (DCM: McOH, 10:1 v/v): R_{f}=0.8. ¹H NMR (400 MHZ, CDCl₃) δ 8.29 (s, 1H), 7.80 (s, 1H), 7.45 (d, J=8.5 Hz, 2H), 7.05 (d, J=8.5 Hz, 1H), 5.56 (s, 1H), 5.38 (d, J=5.0 Hz, 1H), 5.07 (br, 1H), 4.54-4.39 (m, 1H), 3.97 (s, 3H), 3.97 (s, 3H), 3.93 (s, 3H), 3.85-3.54 (m, 6H), 3.39 (br, 2H), 2.41-2.21 (m, 2H), 2.11-0.97 (m, 33H), 0.95 (d, J=6.5 Hz, 3H), 0.90 (dd, J=6.6, 1.9 Hz, 6H), 0.71 (s, 3H). ¹³C NMR (101 MHZ, CDCl₃) δ 167.5, 166.7, 166.2, 165.4, 156.2, 140.7, 140.6, 139.7, 135.3, 134.6, 132.5, 131.4, 130.9, 128.3, 128.1, 122.6, 119.0, 101.8, 74.5, 65.0, 64.4, 56.7, 56.7, 53.1, 53.0, 52.9, 50.0, 42.3, 40.8, 40.1, 39.8, 39.5, 38.5, 37.0, 36.6, 36.2, 35.8, 31.9, 28.3, 28.1, 28.0, 24.3, 23.9, 22.8, 22.6, 21.1, 19.3, 18.7, 11.9. HRMS (m/z): [M+Na]⁺ calcd. for C₅₂H₇₁N₅NaO₁₁, 964.5042; found, 964.5028.

### Synthesis of Compound S7b

The protocol is the same with S7. TLC (Hexane: ethylacetate, 1:1 v/v): Rf=0.5. Purified with silica gel chromatography to give the S7b as off white solid. ¹H NMR (500 MHZ, CDCl₃) 88.16 (s, 1H), 7.68 (s, 1H), 6.00 (t, J=5.8 Hz, 1H), 5.36-5.22 (m, 1H), 4.56 (t, J=6.1 Hz, 1H), 4.45-4.34 (m, 1H), 3.86 (s, 4H), 3.86 (s, 3H), 3.84 (s, 3H), 3.37 (q, J=7.0 Hz, 2H), 3.08 (q, J =6.9 Hz, 3H), 2.35-5.14 (m, 2H), 2.07-0.86 (m, 44H), 0.84 (d, J=6.5 Hz, 3H), 0.79 (dd, J=6.6, 2.3 Hz, 6H), 0.60 (s, 3H). ¹³C NMR (126 MHz, CDCl₃) δ 167.34, 166.81, 166.20, 165.56, 156.19, 140.87, 139.86, 135.30, 132.28, 131.43, 130.91, 128.23, 122.46, 74.19, 56.70, 56.14, 53.10, 53.02, 52.98, 50.01, 42.32, 40.80, 40.23, 39.74, 39.52, 38.58, 36.99, 36.57, 36.19, 35.80, 31.91, 31.88, 29.91, 29.71, 29.28, 28.83, 28.24, 28.18, 28.02, 26.77, 26.57, 24.29, 23.84, 22.83, 22.57, 21.04, 19.34, 18.72, 11.86. HRMS (m/z): [M+H]⁺ calcd. for C₄₈H₇₃N₂O₉, 821.5311; found, 821.5321.

### Synthesis of Compound 2

To a solution of S7 (28 mg, 0.076 mmol, 1.0 equiv) in THF/H2O (4 mL +2 mL) was added lithium hydroxide (4.1 mg, 0.456 mmol, 6.0 equiv) at room temperature. The reaction was stirred for 3 hours, and the solvent was removed under reduced pressure. The resulting solid was redissolved in a small amount of water and purified on a reverse phase C-18 column to generate compound 2 as a white powder (27 mg, quantitative yield). ¹H NMR (500 MHZ, D₂O/CD₃OD=5:1) ¿ 7.55 (s, 1H), 7.41 (s, 1H), 7.34 (br, 2H), 6.84 (br, 2H), 5.46 (br, 1H), 5.22 (br, 1H), 4.22 (br, 1H), 3.84-2.28 (m, 8H), 2.42-0.4 (m, 43H). ¹³C NMR (126 MHz, D2O) δ 176.11, 175.58, 174.64, 171.92, 170.31, 168.30, 139.65, 139.42, 138.17, 137.92, 135.05, 134.22, 128.20, 126.79, 126.16, 118.50, 115.21, 101.36, 73.81, 63.92, 56.69, 56.56, 56.32, 42.24, 39.52, 36.30, 31.58, 29.02, 27.90, 24.34, 23.15, 22.66, 22.46, 18.97, 18.74, 11.87. HRMS (m/z): [M−H]⁻ calcd. for C₄₉H₆₄N₅O₁₁, 898.4608; found, 898.4611.

### Synthesis of Compound 6

The protocol is the same as S7. HRMS (m/z): [M−H]⁻ calcd. for C₄₅H₆₅N₂O₉, 777.4694; found, 777.4696.

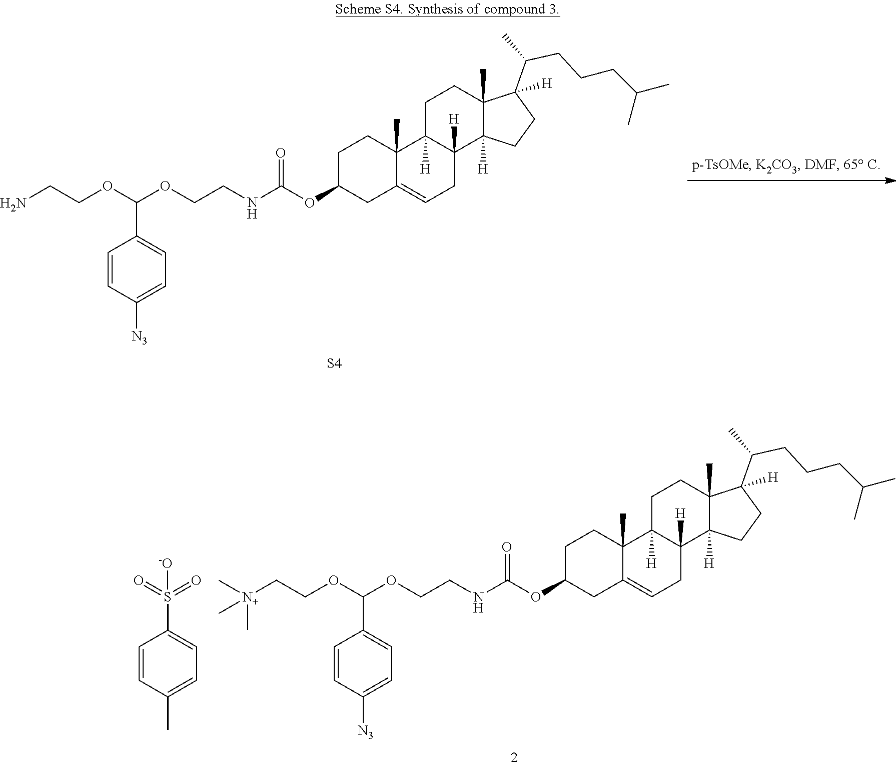

To a solution of S4 (100 mg, 0.151mmol, 1.0 equiv) in DMF (3.0 mL) was added methyl p-toluenesulfonate (112 μL, 0.75 mmol, 5 equiv), followed by sodium carbonate (80 mg, 0.75 mmol, 5 equiv). The reaction mixture was stirred at 65° C. overnight. The reaction residue was filtrated to remove solids and the solvent was removed under reduced pressure. The resulting residue was purified by preparative TLC to afford compound 3 (105 mg, 80%) as a white powder. TLC (DCM: MeOH, 8:1 v/v): Rf=0.4. ¹H NMR (400 MHZ, CDCl₃) δ 7.76 (d, J=7.4 Hz, 2H), 7.38 (d, J=8.3 Hz, 2H), 7.16 (d, J=7.4 Hz, 2H), 7.03 (d, J=8.3 Hz, 2H), 5.54 (s, 1H), 5.46 (br, 1H), 5.38 (br, 1H), 4.48 (dt, J=11.8, 6.7 Hz, 1H), 4.03-3.80 (m, 4H), 3.61 (dt, J=10.3, 5.4 Hz, 1H), 3.55-3.46 (m, 1H), 3.38 (br, 9H for Me, 4H for CH2), 2.43-2.23 (m, 2H), 2.10-1.95 (m, 2H), 1.93-1.81 (m, 2H), 1.66-0.97 (m, 26H), 0.95 (d, J=6.5 Hz, 3H), 0.9 0(dd, J=6.6, 1.8 Hz, 6H), 0.71 (s, 3H). ¹³C NMR (101 MHZ, CDCl₃) δ 156.3, 143.6, 140.9, 139.8, 139.5, 133.9, 128.8, 128.1, 125.8, 122.5, 119.1, 101.7, 65.6, 65.5, 59.6, 56.7, 56.2, 54.3, 50.0, 42.3, 40.6, 39.8, 39.5, 38.6, 37.1, 36.6, 36.2, 35.8, 31.9, 28.2, 28.0, 24.3, 23.9, 22.8, 22.6, 21.3, 21.1, 19.4, 18.7, 11.9. HRMS (m/z): [M]⁺ calcd. for C₄₂H₆₈N₅O₄, 706.5266; found, 706.5263.

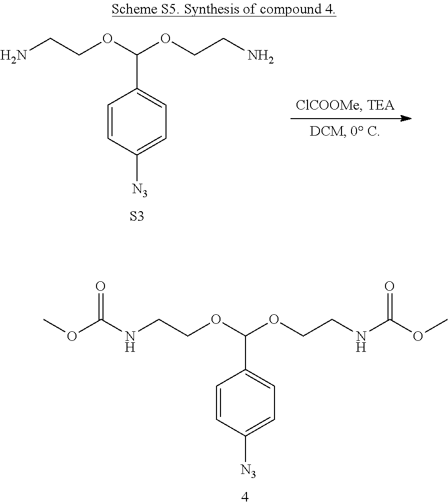

To a solution of S3 (100 mg, 0.398 mmol, 1.0 equiv) and TEA (166 μL, 1.194 mmol, 3equiv) in DCM (10 mL) on an ice bath, was added methyl chloroformate (68 μL, 0.876 mmol, 2.2 equiv). The reaction mixture was stirred in the ice bath for 1 hour. The solvent was removed under reduced pressure and the resulting residue was purified on a Biotage Selekt machine using ethyl acetate/hexane (0.1% TEA) as the eluent and generated 4 (105 mg, 72%) as a white powder. TLC (Hexane: ethylacetate, 2:1 v/v): Rf=0.4.¹H NMR (500 MHZ, CDCl₃) δ 7.33 (d, J =8.5 Hz, 2H), 6.95 (d, J=8.6 Hz, 2H), 5.41 (s, 1H), 5.12 (bs, 2H), 3.59 (s, 6H), 3.42-3.54 (m, 4H), 3.37-3,21 (m, 4H). ¹³C NMR (126 MHZ, CDCl₃) δ 157.1, 140.5, 134.6, 128.1, 119.0, 101.5, 64.8, 52.1, 41.0. HRMS (m/z): [M+Na]⁺ calcd. for C₁₅H₂₁N₅NaO₆, 390.1379; found, 390.1380.

TABLE S1
Hydrolysis kinetics of compound 4 and compound 5.

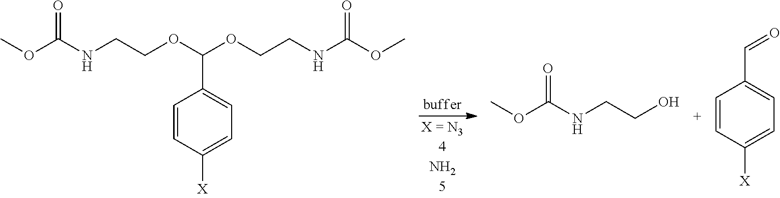

| | t_{1/2} (minute) | | | |
|---|---|---|---|---|
| Compound | pH 4.0 | pH 5.0 | pH 6.0 | pH 7.4 |
| 4 | 3.4 ± 0.08 | 37.12 ± 0.69 | 514.32 ± 7.03 | 21.13 ± 2.01 days |
| 5 | <1.0 | 3.88 ± 0.69 | 14.79 ± 1.37 | 118.96 ± 4.17 |

TABLE S2
Hydrolysis kinetics of compounds 1-3.

| | t_{1/2} (minute) | | | |
|---|---|---|---|---|
| Compound | pH 4.0 | pH 5.0 | pH 6.0 | pH 7.4 |
| Reduced-1 | 1.10 ± 0.15 | 1.72 ± 0.30 | 10.92 ± 0.45 | 113.50 ± 4.95 |
| 1 | 54.75 ± 3.89 | 192.00 ± 28.28 | 2.97 ± 0.31 day | 19.82 ± 1.91 day |
| Reduced-2 | 36.2 ± 0.60 | 83.5 ± 2.45 | 392.4 ± 14.96 | 3.46 ± 0.57 day |
| 2 | 128 ± 1.02 | 342.02 ± 28.8 | 442.9 ± 36.1 | 6.26 ± 0.46 day |
| Reduced-3 | 8.80 ± 0.42 | 25.5 ± 2.12 | 104.38 ± 1.95 | 638.50 ± 74.25 |
| 3 | NA | NA | NA | NA |

Each data represents the arithmetic mean ± SD; NA stands for “not analysable” by plate reader.
All the samples containing compound 3 were incubated at 37° C. for 6 hours, a large amount of compound 3 in the buffer (at pH 6 and pH 7.4) was detected by mass spectrum, and nearly no compound 3 was detected at pH 4 and pH 5.

TABLE S3
DTT reduction kinetics of compound 4.

| | t_{1/2} (minute) | | | |
|---|---|---|---|---|
| Compound | 100 mM | 50 mM | 25 mM | 10 mM |
| 4 | 7.21 ± 0.11 | 11.89 ± 1.85 | 14.25 ± 0.50 | 26.38 ± 3.37 |

Each data point represents the arithmetic mean ± SD

Chemical structures I; lipids. a, lipids used in the base formulas: b, Azido-acctal acid-degradable lipids; c, Non-degradable lipids used in the control experiments.

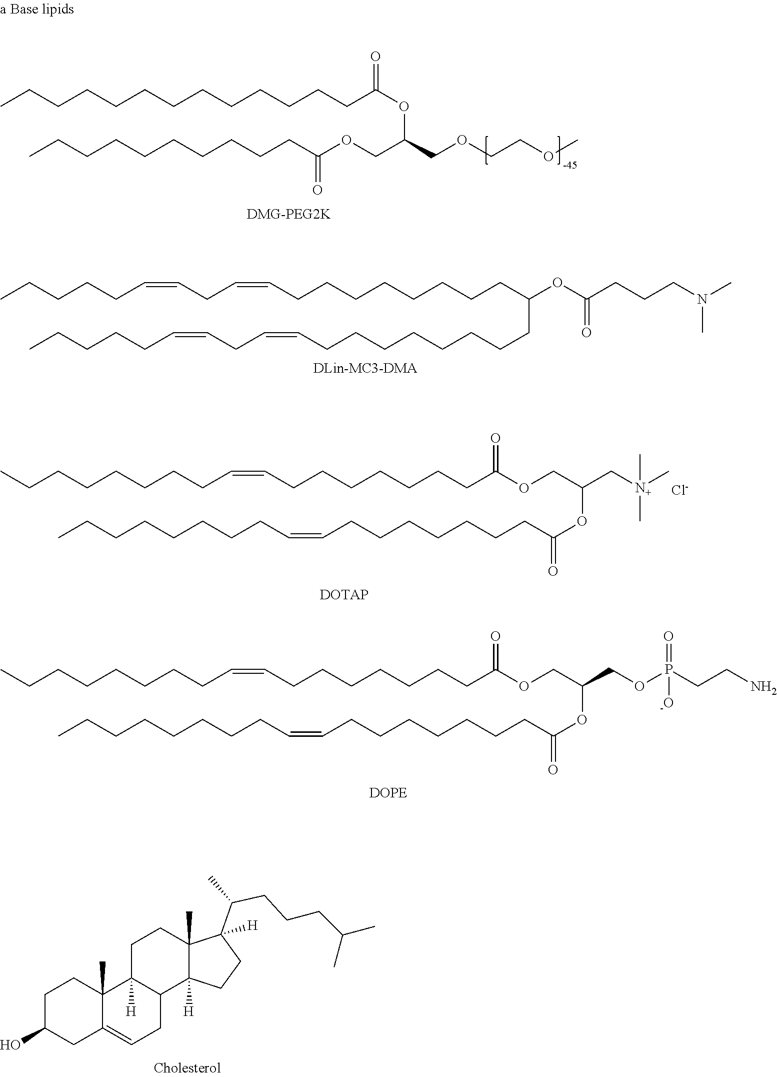

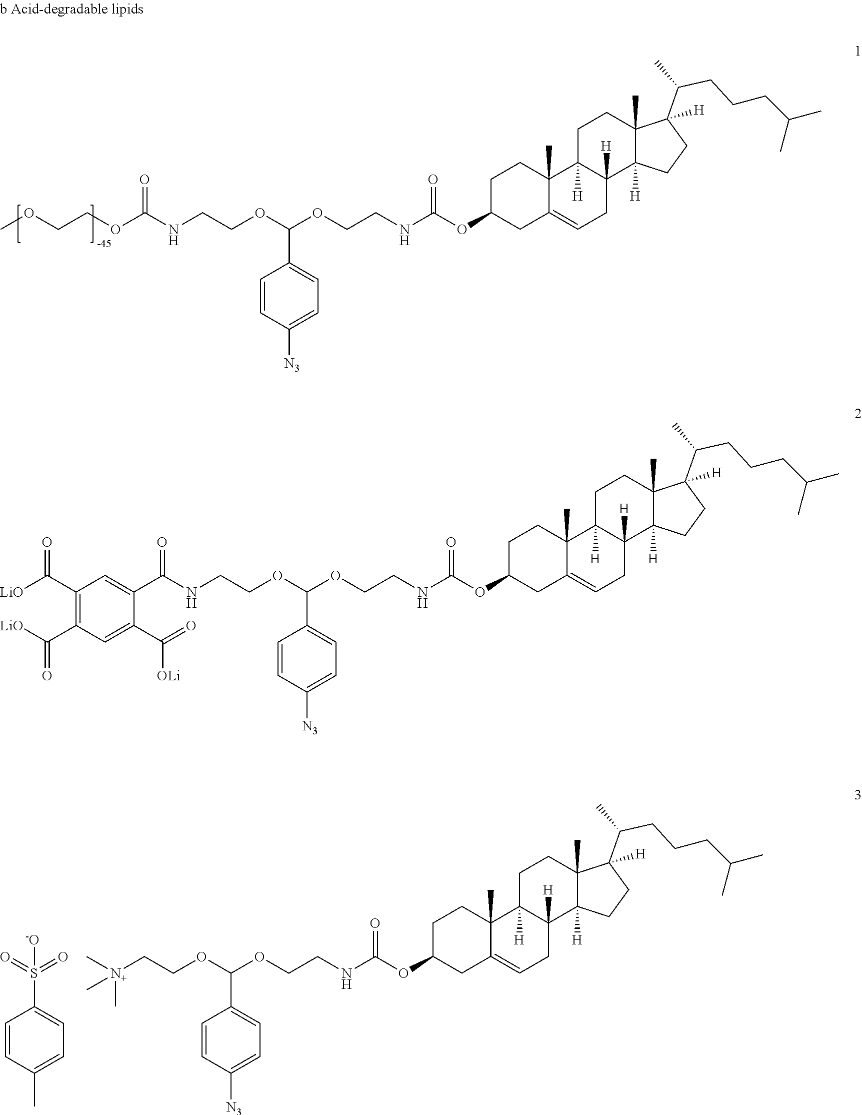

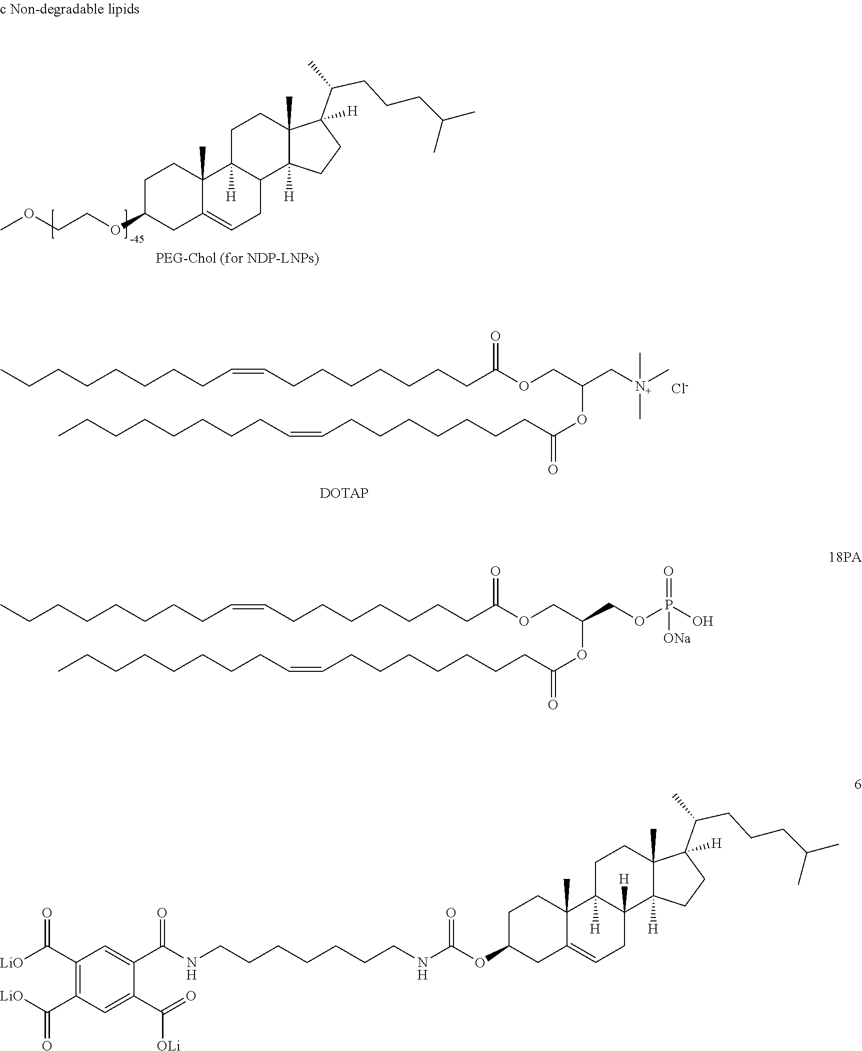

Chemical structures II: azido-acetal lipids after hydrolysis in acidic condition.

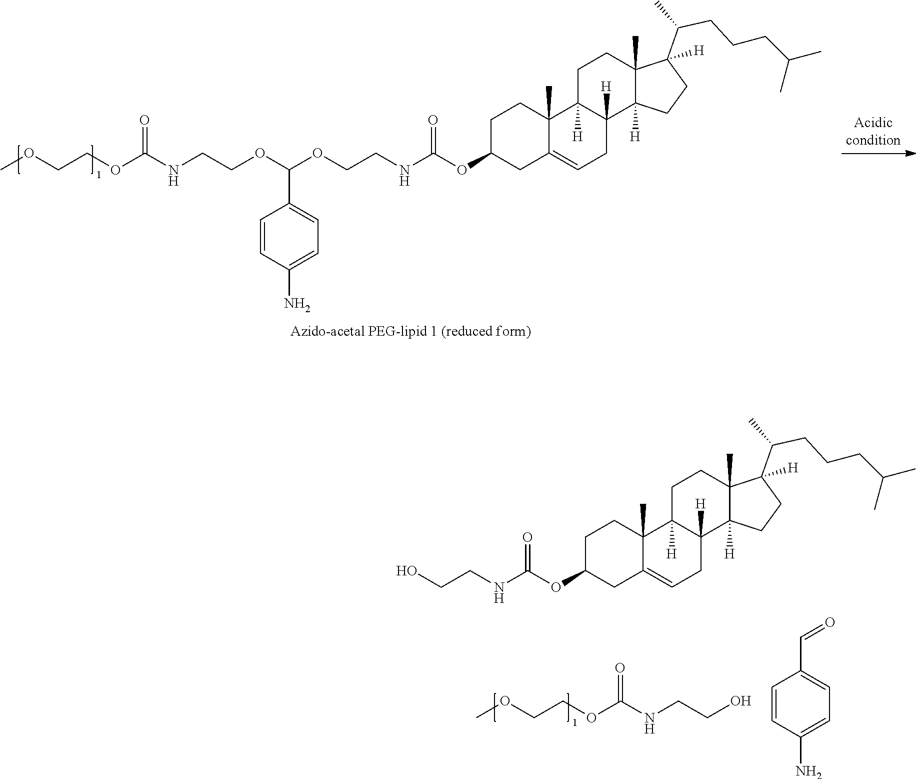

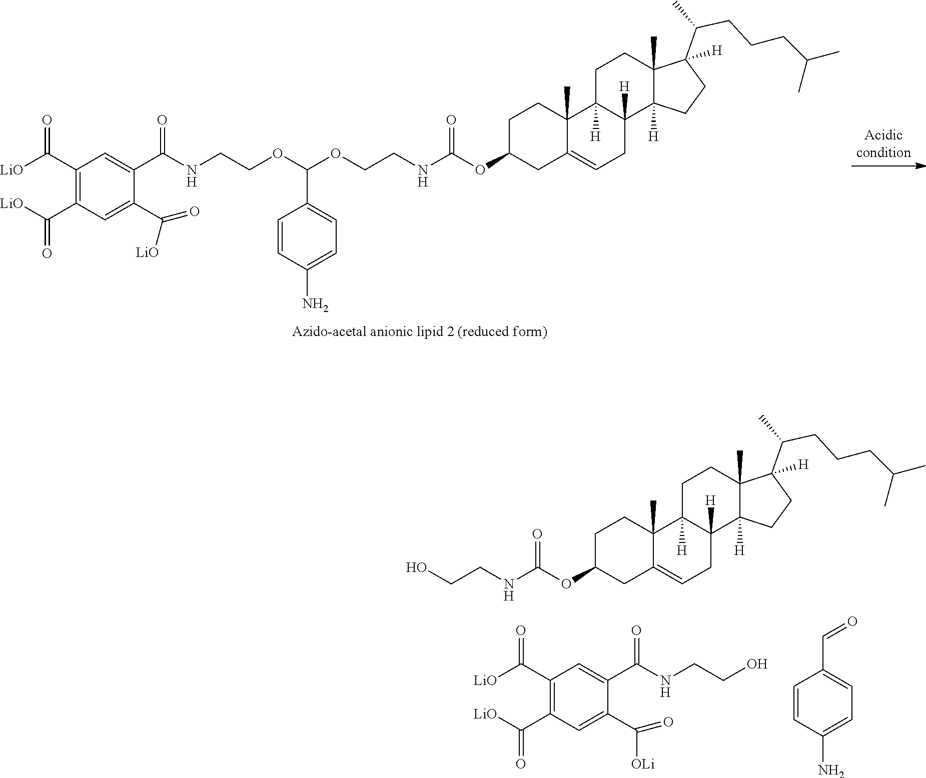

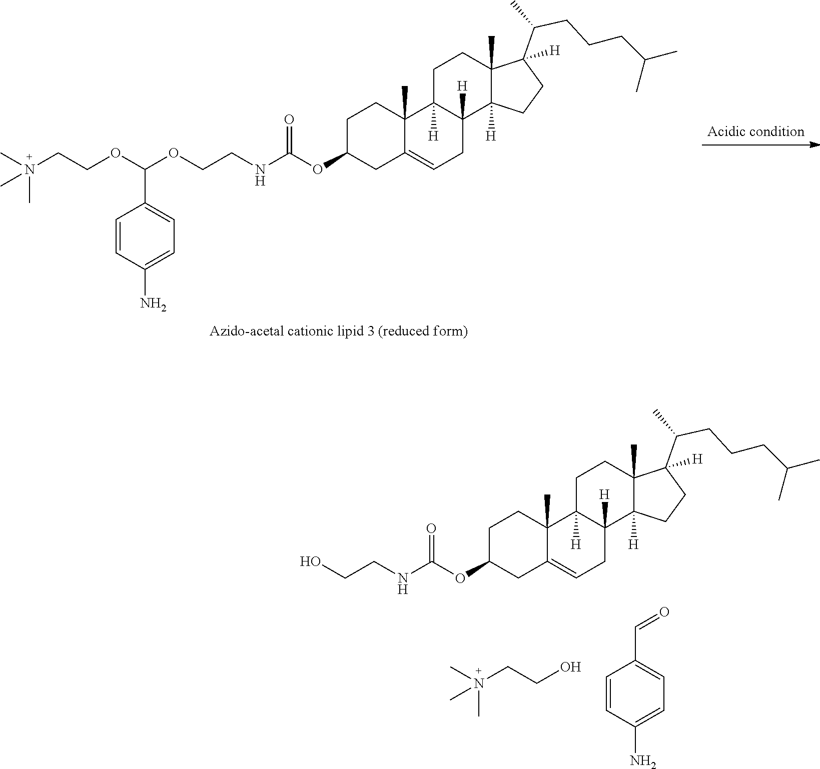

TABLE S4
Components of ADP-LNP.

| PEG- | Molar ratios/Molar percentages (%) | | | | | Size^{b} | ζ | | |
|---|---|---|---|---|---|---|---|---|---|
| lipid | DOTAP | DLin | DOPE | Cholesterol | 1 | (nm) | (mV) | PDI^{b} | E.E. |
| 5% | 35/19.25 | 45/25.06 | 22/11.93 | 70/38.76 | 9.0/5 | 64 | 0.9 | 0.200 | 0.90 |
| 10% | 35/18.24 | 45/23.74 | 22/11.31 | 70/36.71 | 19.0/10 | 61 | 1.5 | 0.225 | 0.89 |
| 20% | 35/16.21 | 45/21.10 | 22/10.05 | 70/32.64 | 42.0/20 | 121 | 2.3 | 0.269 | 0.74 |
| 40% | 35/12.16 | 45/15.82 | 22/7.54 | 70/24.48 | 114/40 | 42, 351^{a} | 3.3 | 0.450 | 0.70 |

^{a}Two distributions were observed;
^{b}The size was obtained from Malvin Zetasizer;
DLin = DLin-MC3-DMA; ζ = zeta potential; E.E. = encapsulation efficiency.

TABLE S5
Components of NDP-LNP.

| | Molar ratios/Molar percentages (%) | | | | | | | | |
|---|---|---|---|---|---|---|---|---|---|
| PEG2K- | | | | | PEG2K- | Size | ζ | | |
| Chol | DOTAP | DLin | DOPE | Cholesterol | Chol | (nm) | (mV) | PDI | E.E |
| 5% | 35/19.25 | 45/25.06 | 22/11.93 | 70/38.75 | 9.05/5 | 116.7 | 5.3 | 0.201 | 0.81 |
| 10% | 35/18.24 | 45/23.74 | 22/11.31 | 70/36.71 | 19.11/10 | 101.9 | 2.6 | 0.250 | 0.83 |
| 20% | 35/16.21 | 45/21.10 | 22/10.05 | 70/32.64 | 42.92/20 | 123 | 2.9 | 0.270 | 0.81 |
| 40% | 35/12.16 | 45/15.82 | 22/7.54 | 70/24.48 | 114.00/40 | 115 | 5.1 | 0.305 | 0.37 |

TABLE S6
Components of ADA-LNP.

| | Molar ratios/Molar percentages (%) | | | | | | | | |
|---|---|---|---|---|---|---|---|---|---|
| Anionic | DMG- | | | | | Size^{a} | ζ | | |
| lipid | PEG2K | DLin | DOTAP | Cholesterol | 2 | (nm) | (mV) | PDI | E.E. |
| 6% | 2.0/1.08 | 62/33.62 | 45/24.41 | 65/34.89 | 11.11/6 | 108.5 | −5.2 | 0.246 | 0.85 |
| 11% | 2.0/1.04 | 62/32.18 | 45/23.38 | 65/33.40 | 19.39/11 | 116.7 | −8.7 | 0.207 | 0.75 |
| 16% | 2.0/0.97 | 62/30.04 | 45/21.81 | 65/31.18 | 33.2/16 | 117.2 | −21.1 | 0.183 | 0.73 |
| | | | | | | | −15.0^{b} | | |

^{a}The size was obtained from Malvin Zetasizer.
^{b}zeta potential was obtained after incubating at pH 5 for 1 hour and dialyzed in PBS at pH 7.4 before measuring the zeta potential.

TABLE S7a
Components of NDA₁-LNP.

| | Molar ratios/Molar percentages (%) | | | | | | | | |
|---|---|---|---|---|---|---|---|---|---|
| | DMG- | | | | | Size | ζ | | |
| 18PA | PEG2K | DLin | DOTAP | Cholesterol | 18PA^{a} | (nm) | (mV) | PDI | E.E. |
| 18% | 2.0/0.95 | 62/29.32 | 45/21.29 | 65/30.44 | 38.32/18 | 97.8 | −2.8 | 0.189 | 0.88 |
| 33% | 2.0/0.77 | 62/23.96 | 45/17.40 | 65/24.87 | 85.41/33 | 105.3 | −2.5 | 0.260 | 0.82 |
| 48% | 2.0/0.60 | 62/18.60 | 45/13.50 | 65/19.30 | 161.13/48 | 117.2 | −4.7 | 0.205 | 0.76 |
| | | | | | | | −4.6^{b} | | |

^{a}The concentration of 18PA was used 3 times to compound 3 in the LNPs because of 3 carboxylic acids in the molecule.
^{b}zeta potential was obtained after incubating at pH 5 for 1 hour and dialyzed in PBS at pH 7.4 before measuring the zeta potential.

TABLE S7b
Components of NDA₂-LNP.

| | Molar ratios/Molar percentages (%) | | | | | | | | |
|---|---|---|---|---|---|---|---|---|---|
| | DMG- | | | | | Size^{a} | ζ | | |
| 6 | PEG2K | DLin | DOTAP | Cholesterol | 6 | (nm) | (mV)^{a} | PDI | E.E. |
| 16% | 2.0/0.97 | 62/30.04 | 45/21.81 | 65/31.18 | 33.2/16 | 115.5 | −19.6 | 0.183 | 0.89 |

^{a}The size and zeta potential were obtained from Malvin Zetasizer.

TABLE S8
Components of ADC-LNP.

| | Molar ratios/Molar percentages (%) | | | | | | | | |
|---|---|---|---|---|---|---|---|---|---|
| Cationic | DMG- | | | | | Size^{a} | ζ | | |
| lipid | PEG2K | DLin | DOPE | Cholesterol | 3 | (nm) | (mV) | PDI | E.E. |
| 5% | 2.0/1.11 | 62/34.96 | 40/22.63 | 65/36.30 | 8.90/5 | 224.5 | 9.3 | 0.181 | 0.83 |
| 10% | 2.0/1.06 | 62/33.12 | 40/21.43 | 65/34.39 | 18.90/10 | 90.3 | 11.2 | 0.184 | 0.83 |
| 20% | 2.0/0.95 | 62/29.44 | 40/19.05 | 65/30.56 | 42.36/20 | 99.3 | 23.5 | 0.182 | 0.95 |
| 40% | 2.0/0.71 | 62/22.08 | 40/14.29 | 65/22.92 | 112.95/40 | 97.0 | 26.8 | 0.206 | 0.98 |

^{a}The size was obtained from Malvin Zetasizer.

TABLE S9
Components of NDC-LNP.

| | Molar ratios/Molar percentages (%) | | | | | | | | |
|---|---|---|---|---|---|---|---|---|---|
| | DMG- | | | | | Size | ζ | | |
| DOTAP | PEG2K | DLin | DOPE | Cholesterol | DOTAP | (nm) | (mV) | PDI | E.E. |
| 5% | 2.0/1.11 | 62/34.96 | 40/22.63 | 65/36.30 | 8.90/5 | 131 | 2.1 | 0.155 | 0.98 |
| 10% | 2.0/1.06 | 62/33.12 | 40/21.43 | 65/34.39 | 18.90/10 | 131 | 3.0 | 0.148 | 0.95 |
| 20% | 2.0/0.95 | 62/29.44 | 40/19.05 | 65/30.56 | 42.36/20 | 111.1 | 5.4 | 0.158 | 0.95 |
| 40% | 2.0/0.71 | 62/22.08 | 40/14.29 | 65/22.92 | 112.95/40 | 124.3 | 6.3 | 0.149 | 0.93 |

TABLE S10
Components of DLin-MC3-DMA-LNP (Std-LNP)

| DLin | Molar ratios/Molar percentages (%) | | | | | | | |
|---|---|---|---|---|---|---|---|---|
| MC3- | PEG2K- | | | | Size^{a} | ζ | | |
| DMA | DMG | DLin | DOPE | Cholesterol | (nm) | (mV)^{a} | PDI | E.E. |
| 36.81% | 2.0/1.18 | 62/36.81 | 40/23.81 | 65/38.20 | 144.0 | −10.3 | 0.139 | 0.92 |

^{a}The size and zeta potential were obtained from Malvin Zetasizer.

TABLE S11
Summary of the pharmacokinetics of ADP-LNPs and Std-LNPs

| | AUC | Clearance | t_{1/2} |
|---|---|---|---|
| | (% ID · h/mL) | 90% (h) | (minute) |
| ADP-LNP | 542.4 ± 39.9 | >24 | 47.0 ± 0.05 |
| Std-LNP | 139.9 ± 12.1 | 0.79 ± 0.18 | 6.18 ± 0.01 |

1. A composition comprising a compound comprising an oxyanion azide-benzaldehyde acetal acid-degradable lipid of structure: 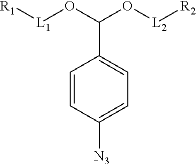
R1 comprises an acyl moiety comprising n (1-5) oxyanions (such as carbonic, phosphoric, sulfonic etc.), each complexed with a metal cation (such as Na⁺, K⁺, Li⁺, etc.);
R2 comprises a hydrophobic group or lipid, such as a steroid (e.g. cholesterol) or one or more alkyl chains, such as in a single chain fatty acid or double or triple chain fatty acid ester, that can strengthen the rigidity of a lipid nanoparticle;
L1 and L2 are linkers selected from a bond, an optionally substituted heteroatom and an optionally substituted C1-18 hydrocarbyl or heterohydrocarbyl, providing acid degradable linkages.

2. The composition of claim 1 wherein R1 comprises a framework of a:
C1-C6 alkyl-substituted heteroatom (N, O or S),
C1-C18 linear or branched alkyl or heteroalkyl,
C3-C6 cycloalkyl or cycloheteroalkyl, or
C5-C6 aryl or heteroaryl.

3. The composition of claim 1 wherein R1 is comprises n (1-5) acetate groups (e.g, of structure -COR(COOM)n).

4. The composition of claim 1, wherein R1 comprises:
C1-C6 alkyl-substituted heteroatom (N, O or S), such as: 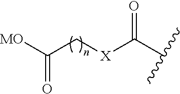
n=1−4, X=N, O, S, such as:

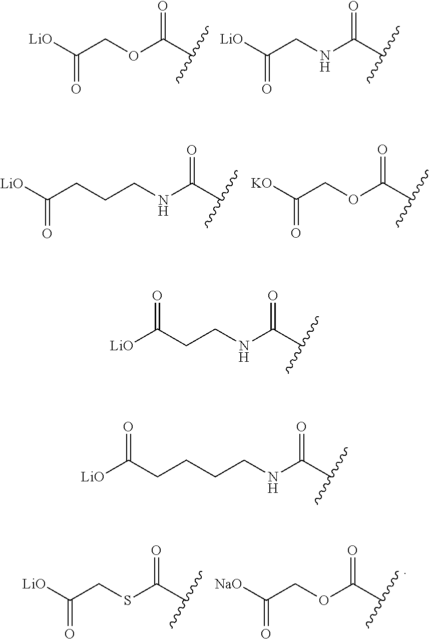

5. The composition of claim 1, wherein R1 comprises:
C1-C18 linear or branched alkyl or heteroalkyl, such as: 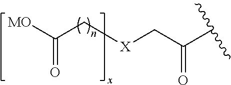
n=1−4, x=1−3, X=N, O, S, such as: 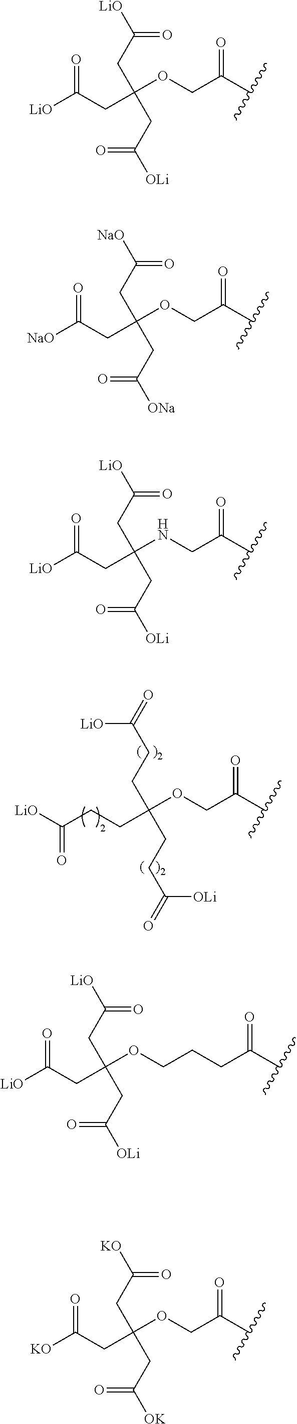 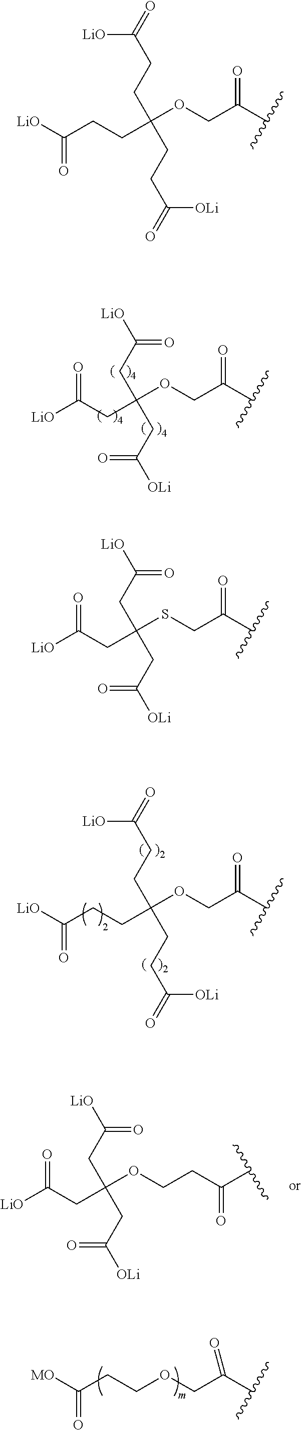
m=1−5, such as 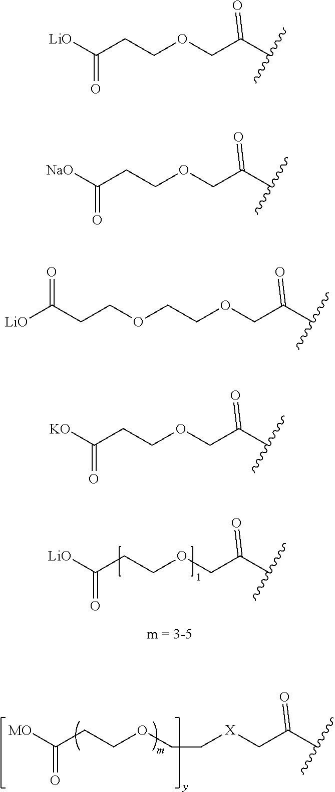
m=1−5, y=2−3, X=N, O, S, such as:

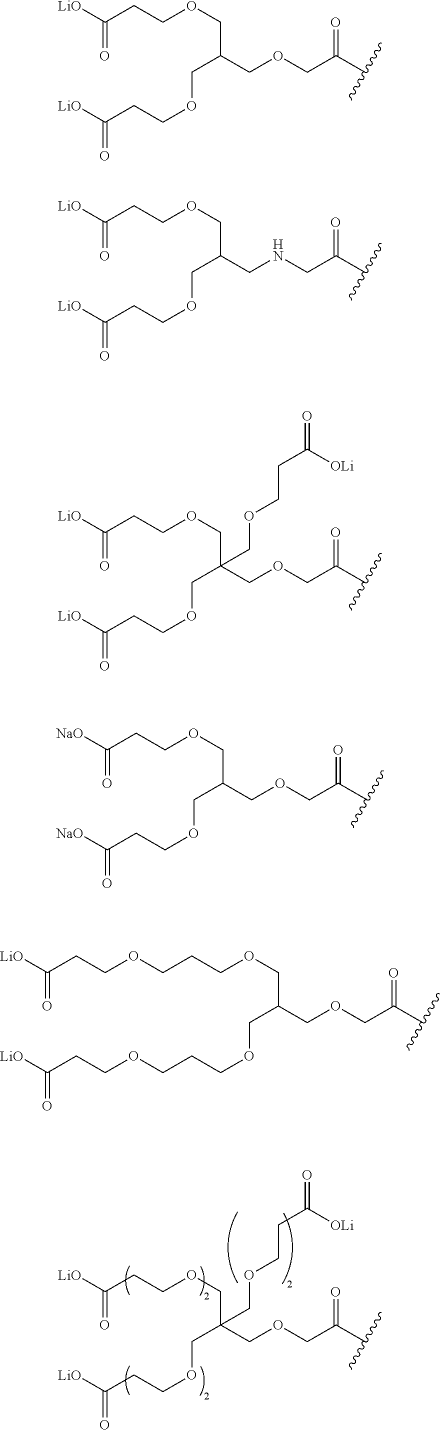

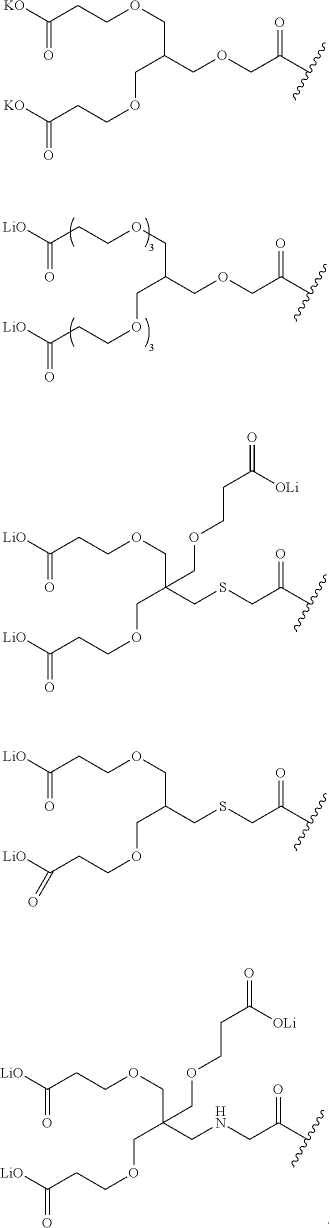

6. The composition of claim 1, wherein R1 comprises:
C3-C6 cycloalkyl or cycloheteroalkyl, such as: cyclohexyl.

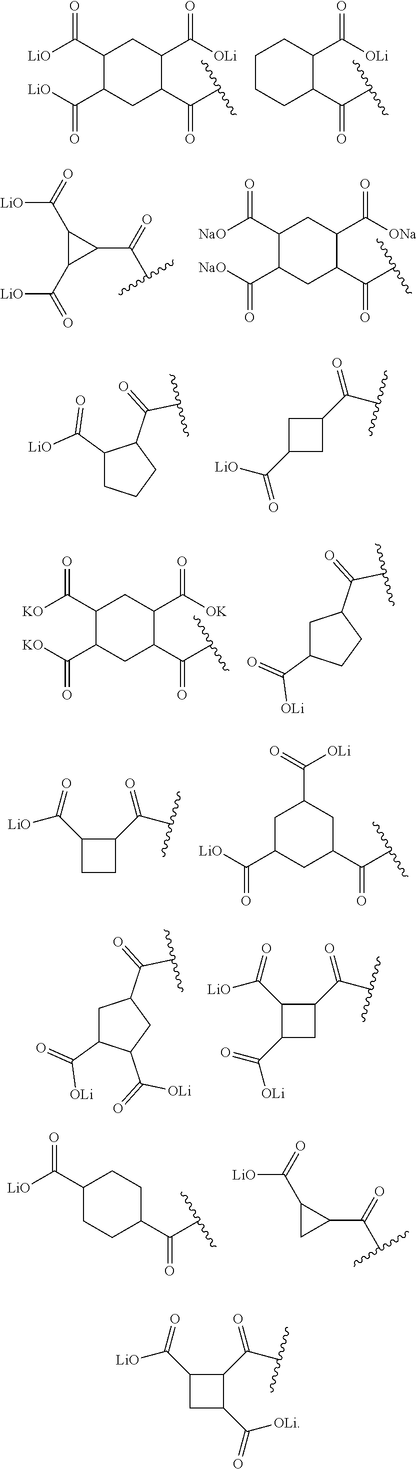

7. The composition of claim 1, wherein R1 comprises:
C5-C6 aryl or heteroaryl, such as:
phenyl. pyridinyl, diazine (e.g. 2. 3 or 4-pyrindinyl. 3.5 or 3.6-diazinyl)

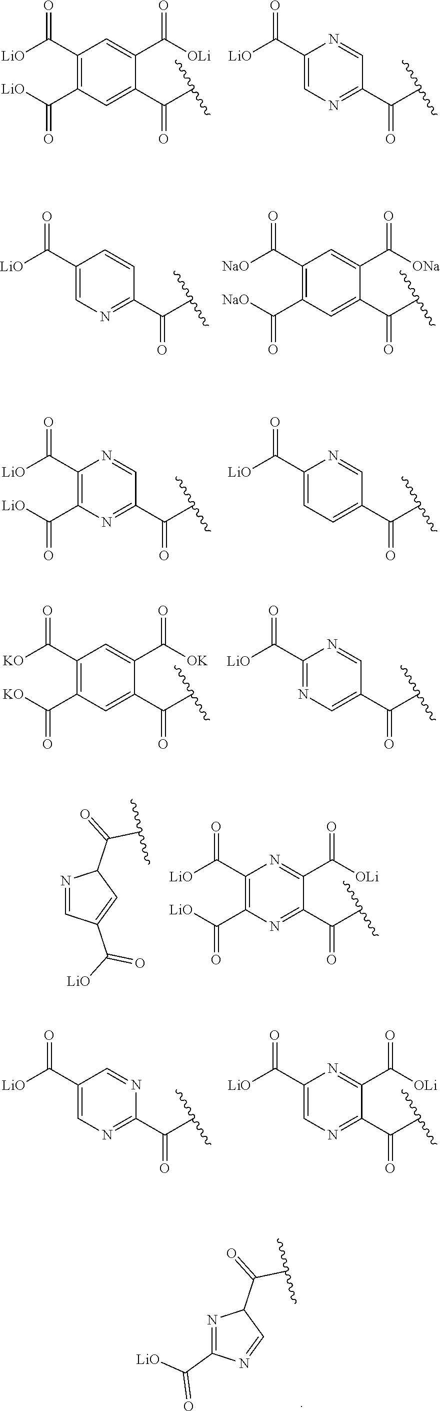

8. The composition of claim 1, wherein R1 comprises: 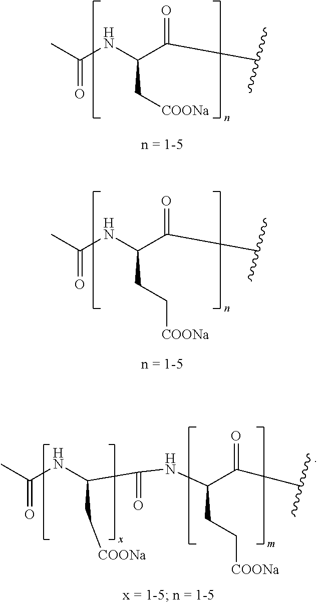
Two types of amino acids can be mixed in the sequence. 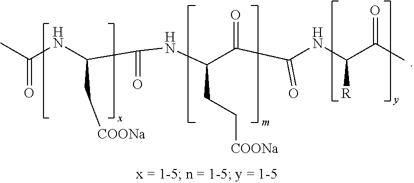
R stands for amino acid residue.
Three types of amino acids can be mixed in the sequence.

9. The composition of claim 1, wherein R1 comprises:

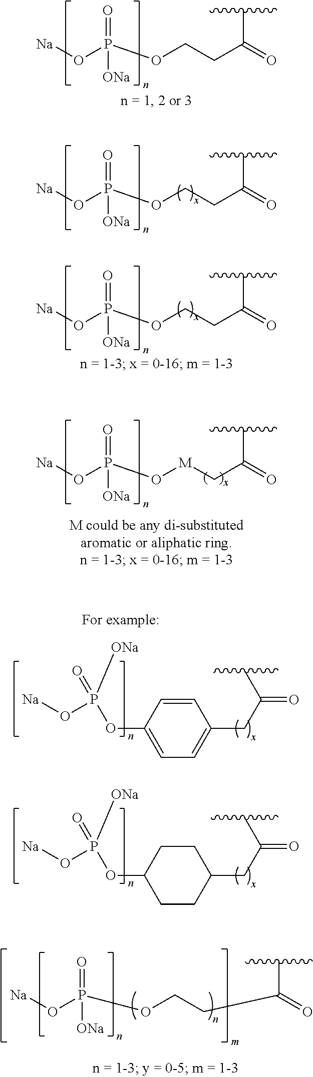

10. The composition of claim 1, wherein R1 comprises:

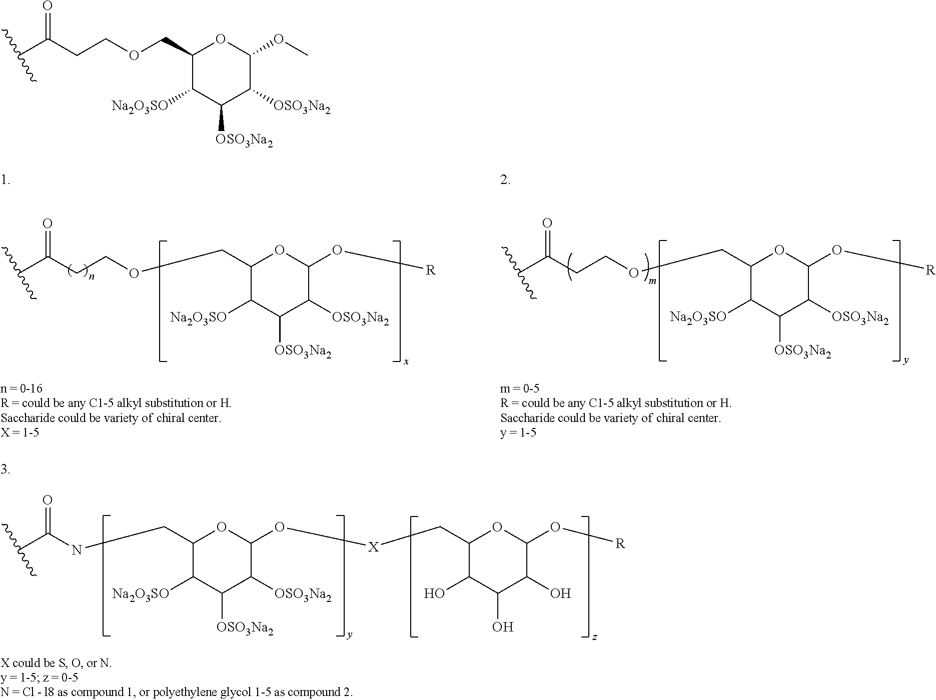

11. A composition of claim 1, wherein:
R2 comprises steroid selected from:

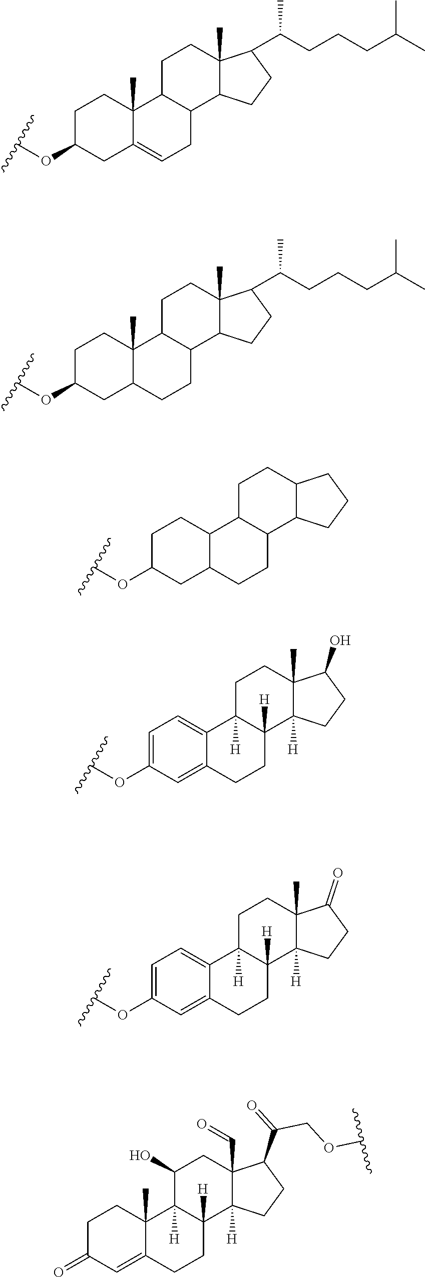

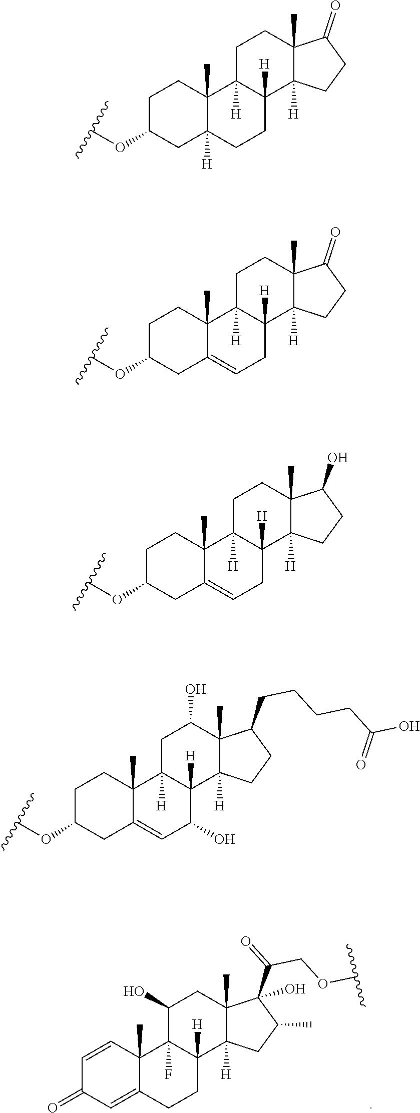

12. A composition of claim 1, wherein R2 comprises an alkyl chain: 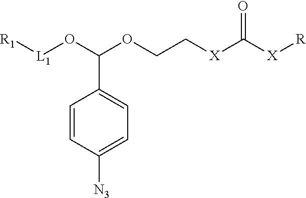
X =O, S, NH, or —CH2— 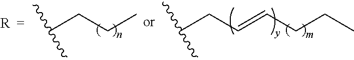
n=4−16; m=1−13; y=1−3, wherein the olefin(s) can be Z/E, and in any position(s) of the chain, such as:

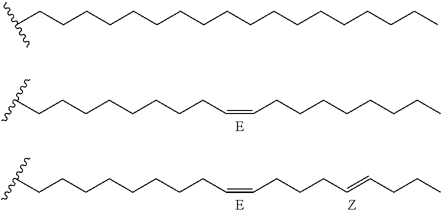 [SMILES: *CCCCCCCCCCCCCCCCCC ; *CCCCCCCC/C=C\CCCCCCCC ; *CCCCCCCC/C=C\CCC/C=C/CCC]

13. A composition of claims 1, wherein R2 comprises an alkyl chain, of structure:

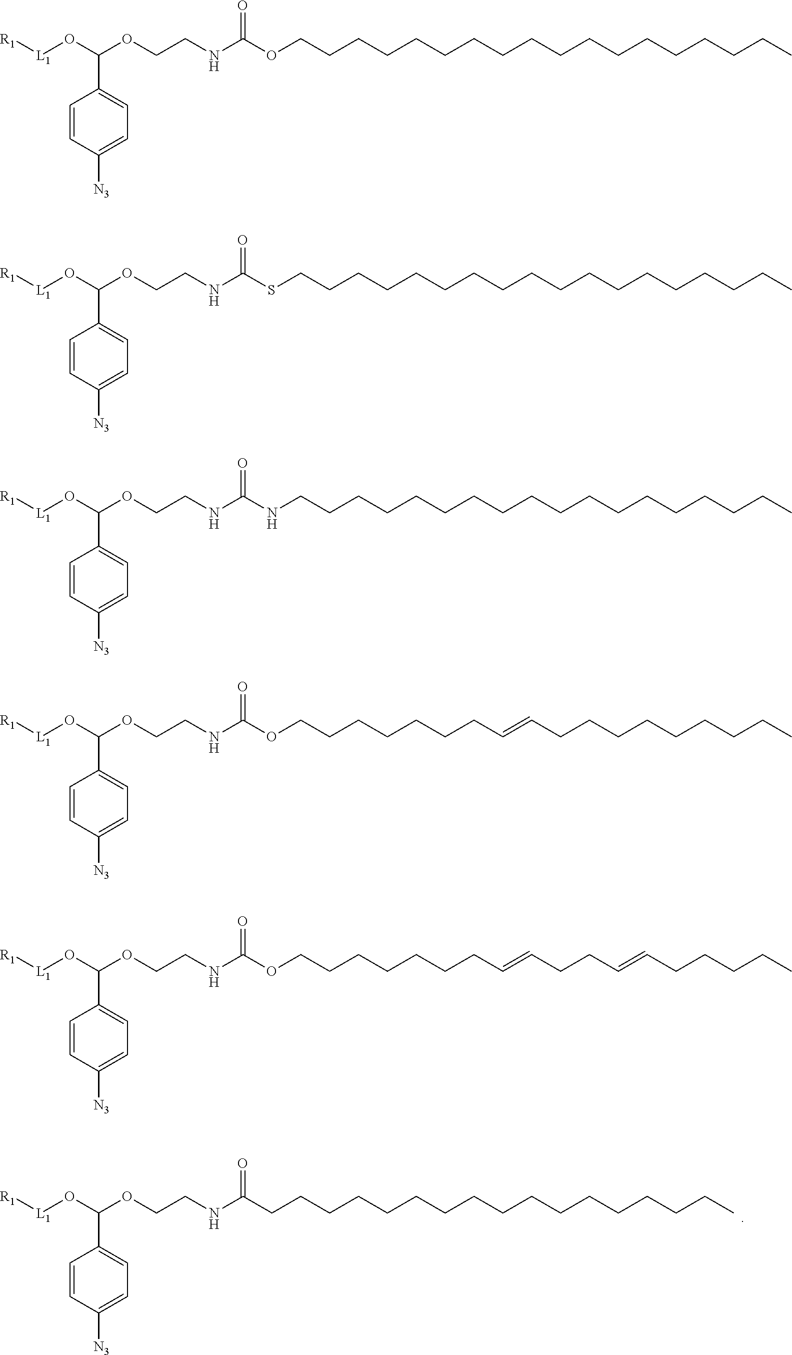

14. A composition of claim 1, wherein R2 comprises two alkyl chains: 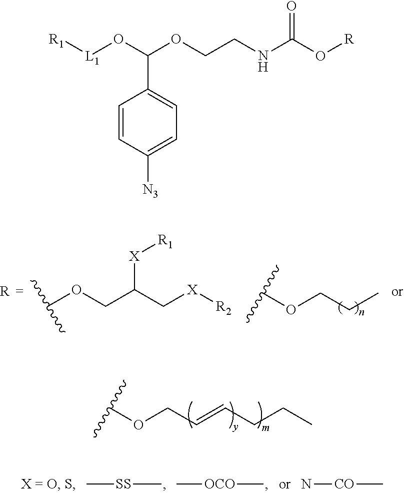
n=4−16; m=1−13; y=1−3, wherein the olefin(s) can be Z/E, and in any position(s) of the chain, such as:

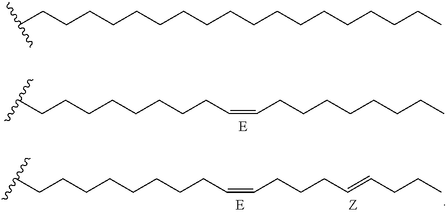 [SMILES: *CCCCCCCCCCCCCCCCCC ; *CCCCCCCC/C=C\CCCCCCCC ; *CCCCCCCC/C=C\CCC/C=C/CCC]

15. A composition of claim 1, wherein R2 comprises two alkyl chains, of structure:

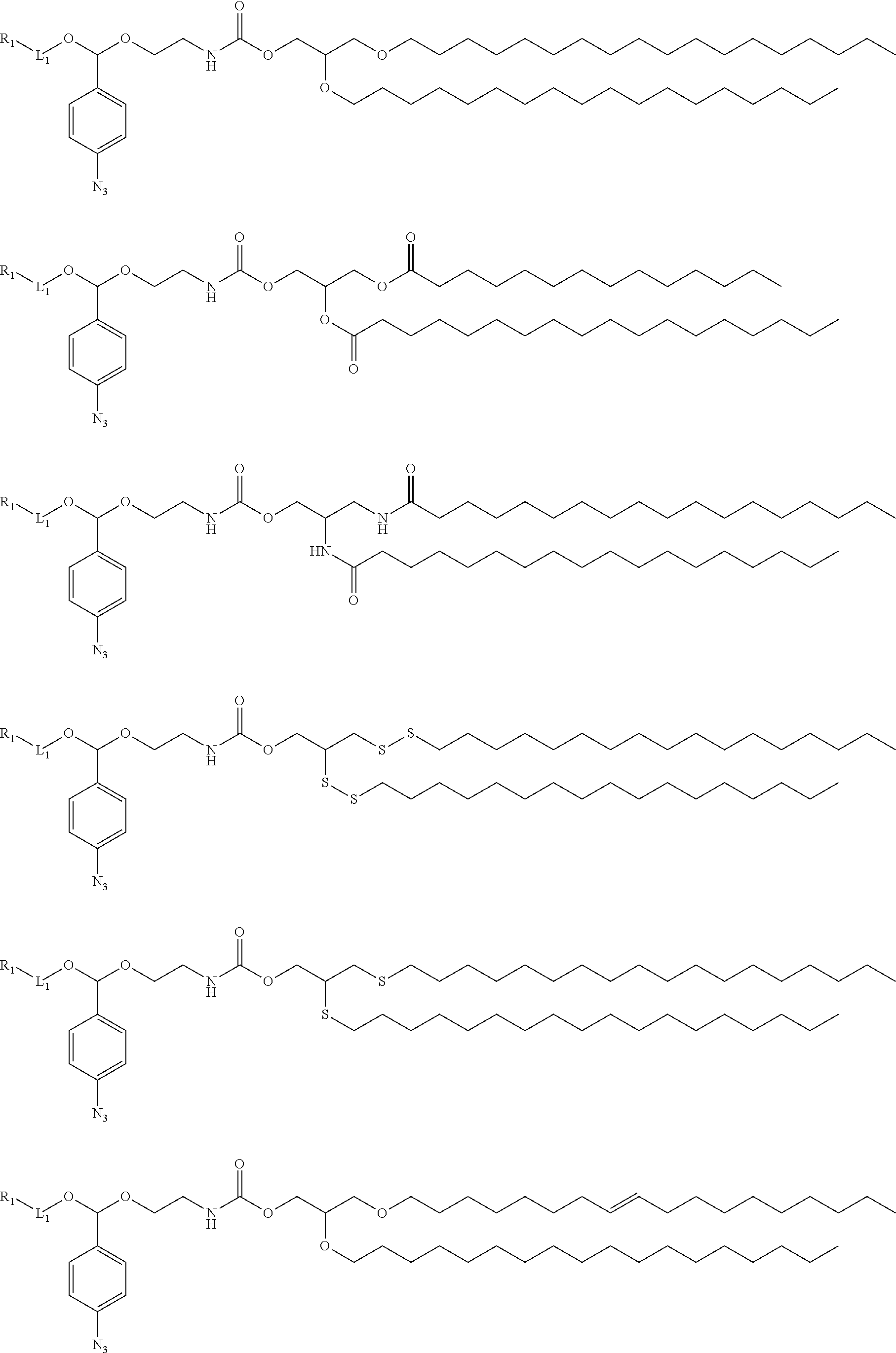

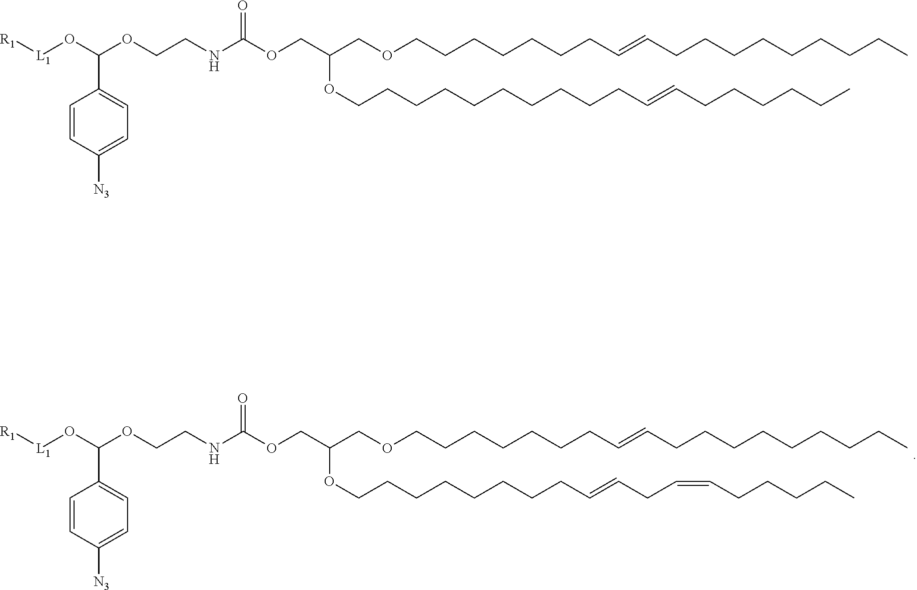

16. A composition of claim 1, wherein R2 comprises three alkyl chains: 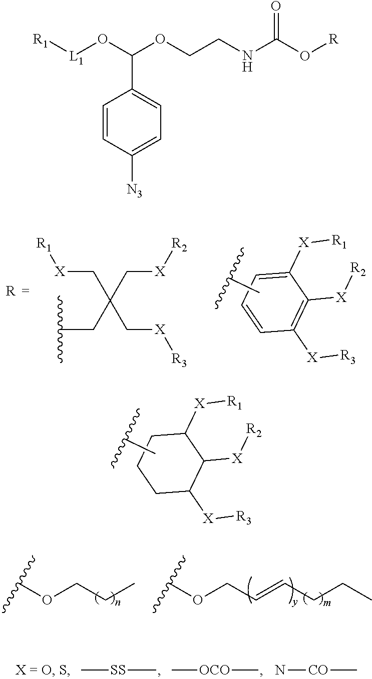
n=4−16; m=1−13; y=1−3, wherein the olefin(s) can be Z/E, and in any position(s) of the chain, such as:

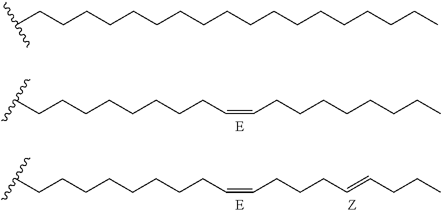 [SMILES: *CCCCCCCCCCCCCCCCCC ; *CCCCCCCC/C=C\CCCCCCCC ; *CCCCCCCC/C=C\CCC/C=C/CCC]

17. A composition of claim 1, wherein R2 comprises three alkyl chains, of structure:

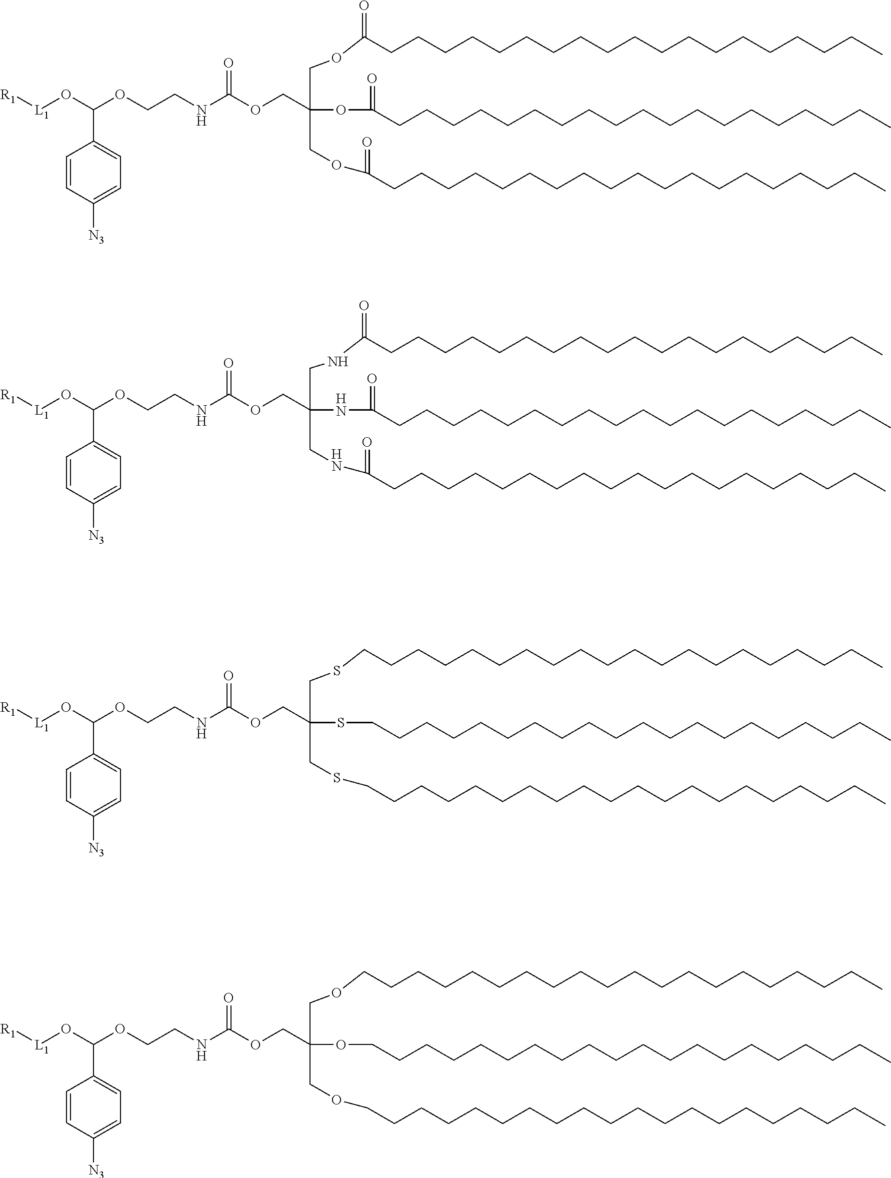

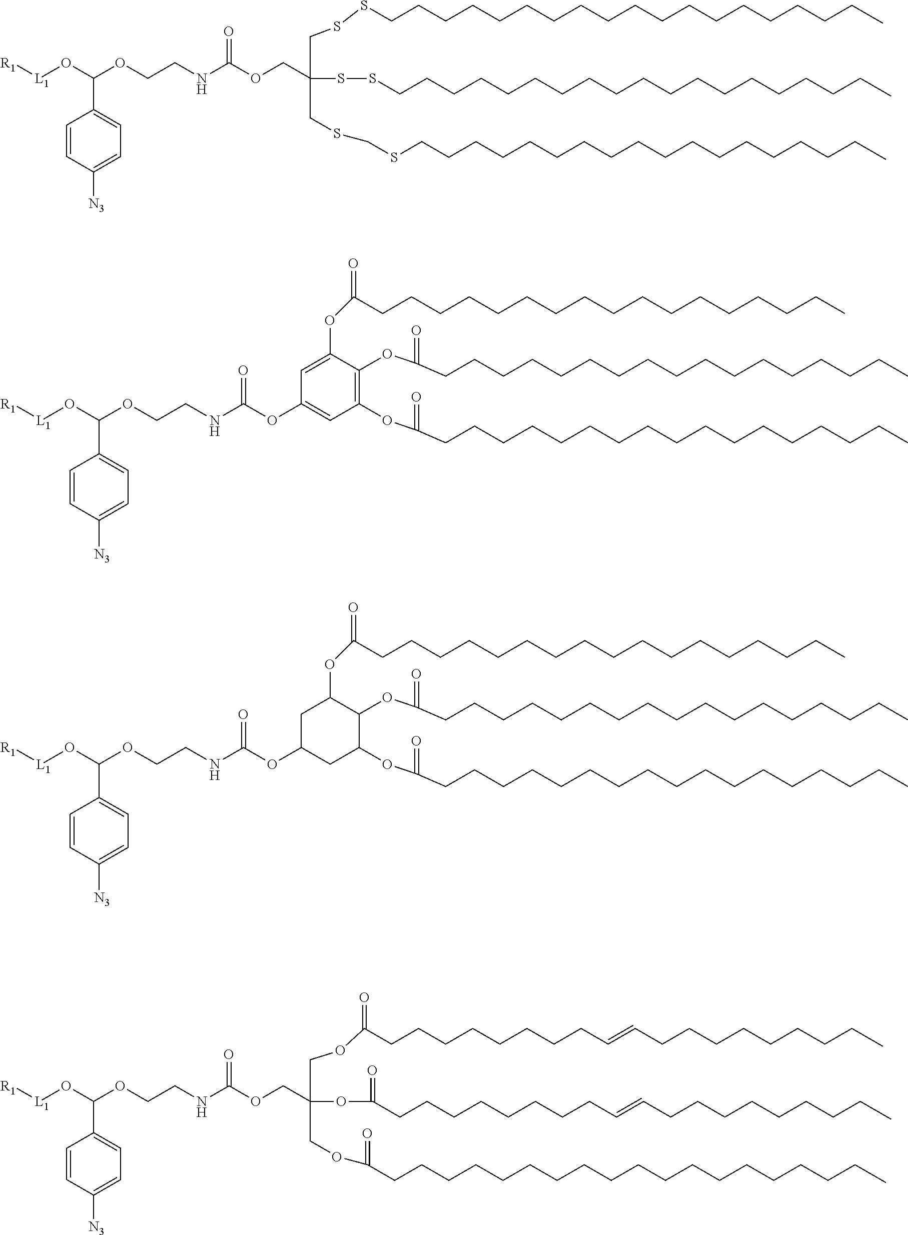

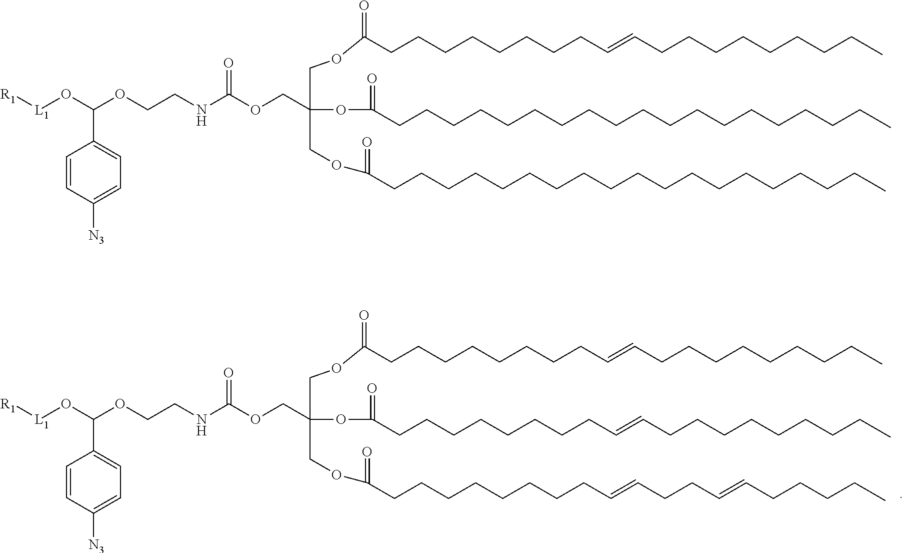

18. A composition of claim 1, wherein:
L1, L2 and L3 comprise linkers independently optionally hetero-, optionally substituted lincar C1-C12 alkyl; or
L1, L2 and L3 comprise linkers independently selected from: 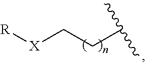
n=1−5; X=N, O, S; or 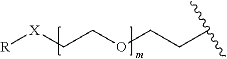
m=0-5, X=N, O, S; or L1 and L2 are —CH2CH2NH-R1 and —CH2CH2NHCO-R2.

19. A composition of claim 1, wherein the azide is reduced to an amine (e.g. prior to injection, for rapid hydrolysis).

20. A lipid nanoparticle (LNP) composition comprising a compound of claim 1, configured, for example, to deliver mRNA, plasma DNA, siRNA, for example, for vaccines, wherein for some of the mRNA, like Cas 9 mRNA may be combined with guide RNA, for cell editing and treating disease.

21. A composition of claim 1, formulated into lipid nanoparticles (LNPs) further comprising a nucleic acid, such as an RNA or DNA, encoding a therapeutic protein, vaccine antigen, or gene editing enzyme(s).

22. A method of using a compound of claim 1, comprising delivering the compounds in a lipid nanoparticle (LNP) composition comprising a compound herein, (a) to transfect a tissue or organ, such as muscle, lung, spleen, liver and blood, or (b) configured as a vaccine or therapeutic, and preferably detecting a resultant intended, targeted effect, and preferably with enhanced effect, e.g. mRNA transfection efficiency attributable to use of the compound.

23. A method of making a compound of claim 1, comprising solid phase peptide synthesis.

24. A solid-phase lipid synthesis method comprising synthesis of cationic, ioniable lipids via solid phase peptide synthesis, and comprising integration in an automated robotic system (ARS) of: (i) the solid phase lipid synthesis, (ii) initial cell screening, and (iii) animal organ or cell targeting.
